SEQUENCE LISTING (21 sequences)

SEQ ID NO: 1 - DNA, 23 bp, synthetic construct
  source 1..23: mol_type = other DNA
aactacatggtgagtgctacatg

SEQ ID NO: 2 - DNA, 18 bp, synthetic construct
  source 1..18: mol_type = other DNA
ctgcccatcagccaggtg

SEQ ID NO: 3 - DNA, 20 bp, synthetic construct
  source 1..20: mol_type = other DNA
actgttggaagtgcatgtgc

SEQ ID NO: 4 - DNA, 21 bp, synthetic construct
  source 1..21: mol_type = other DNA
ttctgagagacctctgcttgg

SEQ ID NO: 5 - DNA, 21 bp, synthetic construct
  source 1..21: mol_type = other DNA
tgagccccaaagctggtgtgg

SEQ ID NO: 6 - DNA, 26 bp, synthetic construct
  source 1..26: mol_type = other DNA
ctgctgctccttgctctcactgcagg

SEQ ID NO: 7 - DNA, 19 bp, synthetic construct
  source 1..19: mol_type = other DNA
ccccaccggaggagttttg

SEQ ID NO: 8 - DNA, 20 bp, synthetic construct
  source 1..20: mol_type = other DNA
gcgaaagccgaagtctaggg

SEQ ID NO: 9 - DNA, 31 bp, synthetic construct
  source 1..31: mol_type = other DNA
  misc_feature 9..10: note = ZEN quencher
cggtctgtagagagctatgcagggtgagggc

SEQ ID NO: 10 - DNA, 20 bp, synthetic construct
  source 1..20: mol_type = other DNA
ccagcttcattttgccctcg

SEQ ID NO: 11 - DNA, 20 bp, synthetic construct
  source 1..20: mol_type = other DNA
ccccttttcagggaatgctc

SEQ ID NO: 12 - DNA, 25 bp, synthetic construct
  source 1..25: mol_type = other DNA
  misc_feature 9..10: note = TAO quencher
ccagcaccccgatgcggacgagtcg

SEQ ID NO: 13 - DNA, 20 bp, synthetic construct
  source 1..20: mol_type = other DNA
taggtcattgctttccggct

SEQ ID NO: 14 - DNA, 20 bp, synthetic construct
  source 1..20: mol_type = other DNA
ttcacagggacggaaaaccg

SEQ ID NO: 15 - DNA, 24 bp, synthetic construct
  source 1..24: mol_type = other DNA
  misc_feature 9..10: note = TAO quencher
ctggctggcgcgcggcgagaactt

SEQ ID NO: 16 - DNA, 20 bp, synthetic construct
  source 1..20: mol_type = other DNA
ggagaaaagcctaccgcaca

SEQ ID NO: 17 - DNA, 20 bp, synthetic construct
  source 1..20: mol_type = other DNA
tgacgtcacccgttctgttg

SEQ ID NO: 18 - DNA, 25 bp, synthetic construct
  source 1..25: mol_type = other DNA
  misc_feature 9..10: note = ZEN quencher
accggcaggtggccccaccctgcat

SEQ ID NO: 19 - DNA, 18 bp, synthetic construct
  source 1..18: mol_type = other DNA
tgttgcggcaagatgcgt

SEQ ID NO: 20 - DNA, 21 bp, synthetic construct
  source 1..21: mol_type = other DNA
ttcattcccactgaccagatg

SEQ ID NO: 21 - DNA, 20 bp, synthetic construct
  source 1..20: mol_type = other DNA
tcccagagcttgcccagagc

(19) United States
(12) Patent Application Publication
(10) Pub. No.: US 2023/0348985 A1
(43) Pub. Date: Nov. 2, 2023

(54) METHODS FOR NON-INVASIVELY MONITORING ORGAN HEALTH IN CROSS-SPECIES TRANSPLANTATION

(71) Applicant: CareDx, Inc., Brisbane, CA (US)

(72) Inventors: Kim CLARK-LANGONE, Redwood City, CA (US); Natali Gulbahce, San Francisco, CA (US); Rongdian Fu, El Cerrito, CA (US); Shelly Shih, Brisbane, CA (US)

(73) Assignee: CarexDx, Inc., Brisbane, CA (US)

(21) Appl. No.: 18/187,638

(22) Filed: Mar. 21, 2023

Related U.S. Application Data
(60) Provisional application No. 63/322,144, filed on Mar. 21, 2022.

(51) Int. Cl. C12Q 1/6883; G16H 50/20; C12Q 1/6888; G16B 30/20

(52) U.S. Cl. CPC C12Q 1/6883; G16H 50/20; C12Q 1/6888; G16B 30/20; C12Q 2600/106; C12Q 2600/16; C12Q 2600/158

(57) ABSTRACT

The present disclosure relates to methods for detecting, predicting, diagnosing and/or monitoring the status of a cross-species transplant in a transplant recipient, as well as to methods for monitoring, administering and adjusting immunosuppressive therapies.

### CROSS-REFERENCE TO RELATED APPLICATIONS

This application claims the benefit of U.S. Provisional Application No. 63/322,144, filed Mar. 21, 2022, which is hereby incorporated by reference in its entirety.

### REFERENCE TO AN ELECTRONIC SEQUENCE LISTING

The contents of the electronic sequence listing (506612003000SEQLIST.xml; Size: 21,075 bytes; and Date of Creation: Mar. 21, 2023) are herein incorporated by reference in their entirety.

### TECHNICAL FIELD

Provided herein are methods and kits for detecting, predicting, diagnosing and/or monitoring the status of cross-species transplants of organs, cells, or tissues, as well as methods of treatment related thereto.

### BACKGROUND

Cross-species transplantation or xenotransplantation, which is the transplantation of cells, tissues or organs from one species into another species, is a promising solution to the severe shortage of human organs, cells and tissues available for transplantation. However, cross-species transplantation presents challenges due, at least in part, to the possibility of cross-species transmission of infections, immune rejection by the transplant recipient, and genetic, anatomical and physiological differences between the donor and recipient. Immune rejection of cross-species transplants can be mediated through both acquired immunity and innate immunity in the transplant recipient. In some cases, the recipient's immune response to cross-species transplantation can lead to hyperacute rejection, acute antibody-mediated rejection, acute cellular rejection, or mixed antibody-mediated and cellular rejections. See, e.g., Lu et al. “Xenotransplantation: Current Status in Preclinical Research.” Frontiers in immunology vol. 10 3060. 23 Jan. 2020. Thus, to prevent or inhibit immune rejection of cross-species transplants, it is necessary to medically suppress the recipient's immune system.

Following transplantation, the status of a transplant in a transplant recipient may be monitored for the remainder of the recipient's lifetime, including assessment function of the transplant and immune-mediated rejection of the transplant. In heart transplantation, for example, surveillance for rejection may include up to 15 scheduled biopsies within the first year of the transplant to provide specimens of the heart muscle for histologic evaluation by a pathologist. Each biopsy procedure is invasive, stressful, inconvenient, and incumbent of procedural risks for the patient, as well as being expensive. Moreover, the biopsy sampling is extremely localized, so histological abnormalities in any non-biopsied areas of the heart are missed. The grading of biopsies is subjective, and discordance of biopsy findings is common between independent pathologists. Furthermore, biopsies are primarily used for surveillance of transplant rejection within the first year after transplantation, but this invasive method is not well suited or established for guiding individualized immunosuppressive therapy in the longer term (e.g., beyond one year after transplant). Clinical laboratory tests have been developed to assess the status of allotransplants, i.e., transplants among members of a single species, based on genetic variations, e.g., single nucleotide polymorphisms (SNPs) or human leukocyte antigen (HLA) genes-based polymorphisms, within the same species. However, these laboratory tests may not be applicable or well-suited to assess the status of a transplant where the donor and the recipient belong to different species.

Thus, there exists a need for improved non-invasive methods of detecting, predicting, diagnosing and/or monitoring the status of a cross-species transplant in a transplant recipient, as well as for methods of determining the need to administer or adjust immunosuppressive therapy being administered to a transplant recipient of a cross-species transplant.

### BRIEF SUMMARY

In one aspect, provided herein is a method for detecting and monitoring transplant rejection status of a transplant from a donor in a transplant recipient, wherein the donor and the recipient belong to different species, the method comprising: a) providing a sample from the transplant recipient post-transplant comprising cell-free nucleic acids that are derived from the donor and cell-free nucleic acids that are derived from the recipient; b) determining an amount of donor-derived cell-free nucleic acids in the sample in a high-throughput sequencing assay by generating sequence reads from the cell-free nucleic acids, wherein the generated sequence reads correspond to donor-specific and recipient-specific genome sequences, and mapping the generated sequence reads to at least the donor-specific genome sequences, wherein differences in genome size between donor and recipient are accounted for; and c) detecting transplant rejection if the amount of donor-derived cell-free nucleic acids exceeds a predetermined threshold. In some embodiments, the method furthermore comprises adding a quantitative spike-in nucleic acid control to the sample in step a, wherein sequence reads from the spike-in control are used to determine the absolute amount of donor-derived cell-free nucleic acids. In some embodiments, the determining step comprises mapping the generated sequence reads to donor-specific and recipient-specific genome sequences. In some embodiments, the sequencing reads are generated from regions of selected sizes of the genomes. In some embodiments, size differences between donor genome and recipient genome are accounted for by utilizing average coverage across regions of selected sizes of the genomes, wherein the regions are large enough to have sufficient coverage and wherein background noise is low enough to differentiate donor and recipient genomes. In some embodiments, a region has a size of up to 1M bases, up to 10M bases, or up to 100M bases. In some embodiments, the amount of donor-derived cell free nucleic acids is a percentage of average coverage across regions of selected sizes of the genomes, wherein the regions are large enough to have sufficient coverage and wherein background noise is low enough to differentiate donor and recipient genomes. In some embodiments, the high-throughput sequencing assay comprises a next-generation sequencing assay.

In another aspect, provided herein is a method for treating transplant rejection in a recipient of a transplant from a donor, wherein the donor and the recipient belong to different species, the method comprising: a) providing a sample from the transplant recipient post-transplant comprising cell-free nucleic acids that are derived from the donor and cell-free nucleic acids that are derived from the recipient; b) determining an amount of donor-derived cell-free nucleic acids in the sample in a high-throughput sequencing assay by generating sequence reads from the cell-free nucleic acids, wherein the generated sequence reads correspond to donor-specific and recipient-specific genome sequences, and mapping the generated sequence reads to at least the donor-specific genome sequences, wherein differences in genome size between donor and recipient are accounted for; c) detecting transplant rejection if the amount of donor-derived cell-free nucleic acids exceeds a predetermined threshold; and d) administering an immunosuppressant treatment to the transplant recipient based on the amount of donor-derived cell-free nucleic acids. In some embodiments, the method furthermore comprises adding a quantitative spike-in nucleic acid control to the sample in step a, wherein sequence reads from the spike-in control are used to determine the absolute amount of donor-derived cell-free nucleic acids. In some embodiments, the determining step comprises mapping the generated sequence reads to donor-specific and recipient-specific genome sequences. In some embodiments, the sequencing reads are generated from regions of selected sizes of the genomes. In some embodiments, size differences between donor genome and recipient genome are accounted for by utilizing average coverage across regions of selected sizes of the genomes, wherein the regions are large enough to have sufficient coverage and wherein background noise is low enough to differentiate donor and recipient genomes. In some embodiments, a region has a size of up to 1M bases, up to 10M bases, or up to 100M bases. In some embodiments, the amount of donor-derived cell free nucleic acids is a percentage of average coverage across regions of selected sizes of the genomes, wherein the regions are large enough to have sufficient coverage and wherein background noise is low enough to differentiate donor and recipient genomes. In some embodiments, the high-throughput sequencing assay comprises a next-generation sequencing assay.

In another aspect, provided herein is a method for adjusting immunosuppressive therapy in a recipient of a transplant from a donor, wherein the donor and the recipient belong to different species, the method comprising: a) providing a sample from the transplant recipient post-transplant comprising cell-free nucleic acids that are derived from the donor and cell-free nucleic acids that are derived from the recipient; b) determining an amount of donor-derived cell-free nucleic acids in the sample in a high-throughput sequencing assay by generating sequence reads from the cell-free nucleic acids, wherein the generated sequence reads correspond to donor-specific and recipient-specific genome sequences, and mapping the generated sequence reads to at least the donor-specific genome sequences, wherein differences in genome size between donor and recipient are accounted for; c) detecting transplant rejection if the amount of donor-derived cell-free nucleic acids exceeds a predetermined threshold; and d) adjusting immunosuppressant treatment being administered to the transplant recipient based on the amount of donor-derived cell-free nucleic acids. In some embodiments, the method furthermore comprises adding a quantitative spike-in nucleic acid control to the sample in step a, wherein sequence reads from the spike-in control are used to determine the absolute amount of donor-derived cell-free nucleic acids. In some embodiments, the determining step comprises mapping the generated sequence reads to donor-specific and recipient-specific genome sequences. In some embodiments, the sequencing reads are generated from regions of selected sizes of the genomes. In some embodiments, size differences between donor genome and recipient genome are accounted for by utilizing average coverage across regions of selected sizes of the genomes, wherein the regions are large enough to have sufficient coverage and wherein background noise is low enough to differentiate donor and recipient genomes. In some embodiments, a region has a size of up to 1M bases, up to 10M bases, or up to 100M bases. In some embodiments, the amount of donor-derived cell free nucleic acids is a percentage of average coverage across regions of selected sizes of the genomes, wherein the regions are large enough to have sufficient coverage and wherein background noise is low enough to differentiate donor and recipient genomes. In some embodiments, the high-throughput sequencing assay comprises a next-generation sequencing assay.

In another aspect, provided herein is a method for detecting and monitoring transplant rejection status of a transplant from a donor in a transplant recipient, wherein the donor and the recipient belong to different species, the method comprising: a) providing a sample from the transplant recipient post-transplant comprising cell-free nucleic acids that are derived from the donor and cell-free nucleic acids that are derived from the recipient; b) determining an amount of donor-derived cell-free nucleic acids in the sample by digital PCR assay, wherein the amount of donor-derived cell-free nucleic acids is determined in reference to amount of sample analyzed or as a ratio of donor-derived cell-free nucleic acids to total donor-specific and recipient-specific nucleic acids by digital PCR quantitation of donor-specific nucleic acids and recipient-specific nucleic acids; and c) detecting transplant rejection if the amount of donor-derived cell-free nucleic acids exceeds a predetermined threshold. In some embodiments, the digital PCR assay contains one or more singleplex digital PCR assays. In some embodiments, the digital PCR assay is a multiplex digital PCR assay consisting of two or more PCR assays in a single digital PCR reaction. In some embodiments, a first singleplex digital PCR assay has a single-copy recipient-specific target in a haploid recipient genome, and a second singleplex digital PCR assay has a single-copy or more-copy donor-specific target in a haploid donor genome. In some embodiments, the second singleplex digital PCR assay has a two-copy or more-copy donor-specific target in a haploid donor genome. In some embodiments, the multiplex digital PCR assay has at least one or two single-copy recipient-specific targets in a haploid recipient genome, and at least one or more single-copy or more-copy donor-specific targets in a haploid donor genome. In some embodiments, each PCR assay has a two-copy or more-copy donor-specific target in a haploid donor genome.

In another aspect, provided herein is a method for treating transplant rejection of a transplant from a donor in a transplant recipient, wherein the donor and the recipient belong to different species, the method comprising: a) providing a sample from the transplant recipient post-transplant comprising cell-free nucleic acids that are derived from the donor and cell-free nucleic acids that are derived from the recipient; b) determining an amount of donor-derived cell-free nucleic acids in the sample by digital PCR assay, wherein the amount of donor-derived cell-free nucleic acids is determined in reference to amount of sample analyzed or as a ratio of donor-derived cell-free nucleic acids to total donor-specific and recipient-specific nucleic acids by digital PCR quantitation of donor-specific nucleic acids and recipient-specific nucleic acids; c) detecting transplant rejection if the amount of donor-derived cell-free nucleic acids exceeds a predetermined threshold; and d) administering an immunosuppressant treatment to the transplant recipient based on the amount of donor-derived cell-free nucleic acids. In some embodiments, the digital PCR assay contains one or more singleplex digital PCR assays. In some embodiments, the digital PCR assay is a multiplex digital PCR assay consisting of two or more PCR assays in a single digital PCR reaction. In some embodiments, a first singleplex digital PCR assay has a single-copy recipient-specific target in a haploid recipient genome, and a second singleplex digital PCR assay has a single-copy or more-copy donor-specific target in a haploid donor genome. In some embodiments, the second singleplex digital PCR assay has a two-copy or more-copy donor-specific target in a haploid donor genome. In some embodiments, the multiplex digital PCR assay has at least one or two single-copy recipient-specific targets in a haploid recipient genome, and at least one or more single-copy or more-copy donor-specific targets in a haploid donor genome. In some embodiments, each PCR assay has a two-copy or more-copy donor-specific target in a haploid donor genome.

In another aspect, provided herein is a method for adjusting immunosuppressive therapy in a recipient of a transplant from a donor, wherein the donor and the recipient belong to different species, the method comprising: a) providing a sample from the transplant recipient post-transplant comprising cell-free nucleic acids that are derived from the donor and cell-free nucleic acids that are derived from the recipient; b) determining an amount of donor-derived cell-free nucleic acids in the sample by digital PCR assay, wherein the amount of donor-derived cell-free nucleic acids is determined in reference to amount of sample analyzed or as a ratio of donor-derived cell-free nucleic acids to total donor-specific and recipient-specific nucleic acids by digital PCR quantitation of donor-specific nucleic acids and recipient-specific nucleic acids; c) detecting transplant rejection if the amount of donor-derived cell-free nucleic acids exceeds a predetermined threshold; and d) adjusting immunosuppressant treatment being administered to the transplant recipient based on the amount of donor-derived cell-free nucleic acids. In some embodiments, the digital PCR assay contains one or more singleplex digital PCR assays. In some embodiments, the digital PCR assay is a multiplex digital PCR assay consisting of two or more PCR assays in a single digital PCR reaction. In some embodiments, a first singleplex digital PCR assay has a single-copy recipient-specific target in a haploid recipient genome, and a second singleplex digital PCR assay has a single-copy or more-copy donor-specific target in a haploid donor genome. In some embodiments, the second singleplex digital PCR assay has a two-copy or more-copy donor-specific target in a haploid donor genome. In some embodiments, the multiplex digital PCR assay has at least one or two single-copy recipient-specific targets in a haploid recipient genome, and at least one or more single-copy or more-copy donor-specific targets in a haploid donor genome. In some embodiments, each PCR assay has a two-copy or more-copy donor-specific target in a haploid donor genome.

In some embodiments, which may be combined with any of the preceding aspects or embodiments, the method further comprises testing for the presence of an infectious agent. In some embodiments, the infectious agent is selected from viruses, bacteria, fungi, or parasites. In some embodiments, the infectious agent is a virus selected from Cytomegalovirus, Epstein-Barr virus, Anelloviridae, or BK virus. In some embodiments, which may be combined with any of the preceding aspects or embodiments, the method further comprises conducting one or more gene expression profiling assays in the recipient. In some embodiments, a combination score is calculated based on the amount of donor-derived cell-free nucleic acids in the sample and the results of the gene expression profiling assay.

In another aspect, provided herein is a kit for detecting and monitoring transplant rejection status of a transplant from a donor in a transplant recipient, wherein the donor and the recipient belong to different species, the kit comprising one or more PCR reaction oligonucleotide primers and probe sets that hybridize to donor-specific or recipient-specific target sequences in cell-free nucleic acids from the transplant recipient for digital PCR quantitation of donor-derived cell-free nucleic acids in reference to the amount of sample analyzed or in reference to total cell-free nucleic acids analyzed, and instructions for data analysis to determine an amount of donor-derived cell-free nucleic acids.

In some embodiments, which may be combined with any of the preceding aspects or embodiments, the donor-derived cell-free nucleic acids are DNA. In some embodiments, which may be combined with any of the preceding aspects or embodiments, the donor-derived cell-free nucleic acids are DNA, RNA, mRNA, miRNA, double-stranded DNA, single-stranded DNA, single-stranded DNA hairpins, DNA/RNA hybrids, RNA hairpins, or a combination thereof.

In some embodiments, which may be combined with any of the preceding aspects or embodiments, the amount of donor-derived cell-free nucleic acids is adjusted with a correction factor to correct for differences in genome length between the donor and recipient genomes. In some embodiments, which may be combined with any of the preceding aspects or embodiments, the amount of donor-derived cell-free nucleic acids is adjusted with a correction factor to correct for cell-free nucleic acid fragment size differences between cell-free nucleic acids that are derived from the donor and cell-free nucleic acids that are derived from the recipient.

In some embodiments, which may be combined with any of the preceding aspects or embodiments, the amount of donor-derived cell-free nucleic acids is a ratio of donor-derived cell-free nucleic acids to total or recipient-derived cell-free nucleic acids. In some embodiments, the ratio is a ratio of donor-derived cell-free nucleic acid mass to total or recipient-derived cell-free nucleic acid mass. In some embodiments, the ratio is a ratio of donor-derived cell-free nucleic acid genome copy equivalents to total or recipient-derived cell-free nucleic acid genome copy equivalents.

In some embodiments, which may be combined with any of the preceding aspects or embodiments, the amount of donor-derived cell-free nucleic acids is a percentage of donor-derived cell-free nucleic acids compared to total cell-free nucleic acids. In some embodiments, the percentage is a percentage of donor-derived cell-free nucleic acid mass compared to total cell-free nucleic acid mass. In some embodiments, the percentage is a percentage of donor-derived cell-free nucleic acid genome copy equivalents compared to total cell-free nucleic acid genome copy equivalents.

In some embodiments, which may be combined with any of the preceding aspects or embodiments, the transplant is a solid organ, tissue or cell transplant. In some embodiments, which may be combined with any of the preceding aspects or embodiments, the donor of the transplant is an animal. In some embodiments, the animal is a pig.

In some embodiments, which may be combined with any of the preceding aspects or embodiments, the next-generation sequencing assay is amplicon-based. In some embodiments, which may be combined with any of the preceding aspects or embodiments, the next-generation sequencing assay is non-amplicon based.

In another aspect, provided herein is a method for detecting and monitoring transplant rejection status of a transplant from a donor in a transplant recipient, wherein the donor and the recipient belong to different species, the method comprising: a) providing a sample from the transplant recipient post-transplant, wherein the sample comprises cell-free nucleic acids that are derived from the donor and cell-free nucleic acids that are derived from the recipient; b) determining an amount of donor-derived cell-free nucleic acids in the sample by a PCR assay, wherein the amount of donor-derived cell-free nucleic acids is determined: (i) as absolute copies of donor-derived cell-free nucleic acids in the sample, or (ii) as a ratio of donor-derived cell-free nucleic acids to total donor-derived and recipient-derived cell-free nucleic acids, by PCR quantitation of donor-specific nucleic acids and recipient-specific nucleic acids; and c) detecting transplant rejection if the amount of donor-derived cell-free nucleic acids exceeds a predetermined threshold. In some embodiments, the PCR assay is a quantitative PCR (qPCR) assay and the PCR quantitation is a real time PCR quantitation. In some embodiments, the PCR assay is a digital PCR assay and the PCR quantitation is an endpoint PCR quantitation. In some embodiments, the digital PCR assay comprises: (a) at least a singleplex digital PCR assay for a single-copy or multi-copy donor-specific target, wherein the number of copies of the donor-specific target is in reference to a haploid donor genome; and/or (b) at least a singleplex digital PCR assay for a single-copy or multi-copy recipient-specific target, wherein the number of copies of the recipient-specific target is in reference to a haploid recipient genome. In some embodiments, the digital PCR assay comprises at least a multiplex digital PCR assay for two or more single-copy or multi-copy donor-specific targets and/or recipient-specific targets in a single digital PCR reaction, wherein the number of copies of the donor-specific targets is in reference to a haploid donor genome and the number of copies of the recipient-specific targets is in reference to a haploid recipient genome. In some embodiments, the digital PCR assay comprises a first singleplex digital PCR assay and a second singleplex digital PCR assay, wherein: (a) the first singleplex digital PCR assay is for a single-copy or multi-copy recipient-specific target, wherein the number of copies of the recipient-specific target is in reference to a haploid recipient genome, and (b) the second singleplex digital PCR assay is for a single-copy or multi-copy donor-specific target, wherein the number of copies of the donor-specific target is in reference to a haploid donor genome. In some embodiments, the second singleplex digital PCR assay is for a multi-copy donor-specific target. In some embodiments, the multiplex digital PCR assay is for: (a) at least one single-copy or multi-copy recipient-specific target, wherein the number of copies of the recipient-specific target is in reference to a haploid recipient genome, and (b) at least one single-copy or multi-copy donor-specific target, wherein the number of copies of the donor-specific target is in reference to a haploid donor genome. In some embodiments, the multiplex digital PCR assay is for at least one multi-copy donor-specific target, wherein the number of copies of the donor specific-target is in reference to a haploid donor genome.

In another aspect, provided herein is a method for treating transplant rejection of a transplant from a donor in a transplant recipient, wherein the donor and the recipient belong to different species, the method comprising: a) providing a sample from the transplant recipient post-transplant, wherein the sample comprises cell-free nucleic acids that are derived from the donor and cell-free nucleic acids that are derived from the recipient; b) determining an amount of donor-derived cell-free nucleic acids in the sample by a PCR assay, wherein the amount of donor-derived cell-free nucleic acids is determined: (i) as absolute copies of donor-derived cell-free nucleic acids in the sample, or (ii) as a ratio of donor-derived cell-free nucleic acids to total donor-derived and recipient-derived cell-free nucleic acids, by PCR quantitation of donor-specific nucleic acids and recipient-specific nucleic acids; c) detecting transplant rejection if the amount of donor-derived cell-free nucleic acids exceeds a predetermined threshold; and d) administering an immunosuppressant treatment to the transplant recipient based on the amount of donor-derived cell-free nucleic acids. In some embodiments, the PCR assay is a quantitative PCR (qPCR) assay and the PCR quantitation is a real time PCR quantitation. In some embodiments, the PCR assay is a digital PCR assay and the PCR quantitation is an endpoint PCR quantitation. In some embodiments, the digital PCR assay comprises: (a) at least a singleplex digital PCR assay for a single-copy or multi-copy donor-specific target, wherein the number of copies of the donor-specific target is in reference to a haploid donor genome; and/or (b) at least a singleplex digital PCR assay for a single-copy or multi-copy recipient-specific target, wherein the number of copies of the recipient-specific target is in reference to a haploid recipient genome. In some embodiments, the digital PCR assay comprises at least a multiplex digital PCR assay for two or more single-copy or multi-copy donor-specific targets and/or recipient-specific targets in a single digital PCR reaction, wherein the number of copies of the donor-specific targets is in reference to a haploid donor genome and the number of copies of the recipient-specific targets is in reference to a haploid recipient genome. In some embodiments, the digital PCR assay comprises a first singleplex digital PCR assay and a second singleplex digital PCR assay, wherein: (a) the first singleplex digital PCR assay is for a single-copy or multi-copy recipient-specific target, wherein the number of copies of the recipient-specific target is in reference to a haploid recipient genome, and (b) the second singleplex digital PCR assay is for a single-copy or multi-copy donor-specific target, wherein the number of copies of the donor-specific target is in reference to a haploid donor genome. In some embodiments, the second singleplex digital PCR assay is for a multi-copy donor-specific target. In some embodiments, the multiplex digital PCR assay is for: (a) at least one single-copy or multi-copy recipient-specific target, wherein the number of copies of the recipient-specific target is in reference to a haploid recipient genome, and (b) at least one single-copy or multi-copy donor-specific target, wherein the number of copies of the donor-specific target is in reference to a haploid donor genome. In some embodiments, the multiplex digital PCR assay is for at least one multi-copy donor-specific target, wherein the number of copies of the donor specific-target is in reference to a haploid donor genome.

In another aspect, provided herein is a method for adjusting immunosuppressive therapy in a recipient of a transplant from a donor, wherein the donor and the recipient belong to different species, the method comprising: a) providing a sample from the transplant recipient post-transplant, wherein the sample comprises cell-free nucleic acids that are derived from the donor and cell-free nucleic acids that are derived from the recipient; b) determining an amount of donor-derived cell-free nucleic acids in the sample by a PCR assay, wherein the amount of donor-derived cell-free nucleic acids is determined: (i) as absolute copies of donor-derived cell-free nucleic acids in the sample, or (ii) as a ratio of donor-derived cell-free nucleic acids to total donor-derived and recipient-derived cell-free nucleic acids, by PCR quantitation of donor-specific nucleic acids and recipient-specific nucleic acids; c) detecting transplant rejection if the amount of donor-derived cell-free nucleic acids exceeds a predetermined threshold; and d) adjusting immunosuppressant treatment being administered to the transplant recipient based on the amount of donor-derived cell-free nucleic acids. In some embodiments, the PCR assay is a quantitative PCR (qPCR) assay and the PCR quantitation is a real time PCR quantitation. In some embodiments, the PCR assay is a digital PCR assay and the PCR quantitation is an endpoint PCR quantitation. In some embodiments, the digital PCR assay comprises: (a) at least a singleplex digital PCR assay for a single-copy or multi-copy donor-specific target, wherein the number of copies of the donor-specific target is in reference to a haploid donor genome; and/or (b) at least a singleplex digital PCR assay for a single-copy or multi-copy recipient-specific target, wherein the number of copies of the recipient-specific target is in reference to a haploid recipient genome. In some embodiments, the digital PCR assay comprises at least a multiplex digital PCR assay for two or more single-copy or multi-copy donor-specific targets and/or recipient-specific targets in a single digital PCR reaction, wherein the number of copies of the donor-specific targets is in reference to a haploid donor genome and the number of copies of the recipient-specific targets is in reference to a haploid recipient genome. In some embodiments, the digital PCR assay comprises a first singleplex digital PCR assay and a second singleplex digital PCR assay, wherein: (a) the first singleplex digital PCR assay is for a single-copy or multi-copy recipient-specific target, wherein the number of copies of the recipient-specific target is in reference to a haploid recipient genome, and (b) the second singleplex digital PCR assay is for a single-copy or multi-copy donor-specific target, wherein the number of copies of the donor-specific target is in reference to a haploid donor genome. In some embodiments, the second singleplex digital PCR assay is for a multi-copy donor-specific target. In some embodiments, the multiplex digital PCR assay is for: (a) at least one single-copy or multi-copy recipient-specific target, wherein the number of copies of the recipient-specific target is in reference to a haploid recipient genome, and (b) at least one single-copy or multi-copy donor-specific target, wherein the number of copies of the donor-specific target is in reference to a haploid donor genome. In some embodiments, the multiplex digital PCR assay is for at least one multi-copy donor-specific target, wherein the number of copies of the donor specific-target is in reference to a haploid donor genome.

In some embodiments, which may be combined with any of the preceding aspects or embodiments, the method further comprises testing for the presence of an infectious agent. In some embodiments, the infectious agent is selected from the group consisting of viruses, bacteria, fungi, and parasites. In some embodiments, the infectious agent is a virus selected from the group consisting of Cytomegalovirus, Epstein-Barr virus, Anelloviridae, and BK virus. In some embodiments, which may be combined with any of the preceding aspects or embodiments, the method further comprises conducting one or more gene expression profiling assays in the recipient. In some embodiments, a combination score is calculated based on the amount of donor-derived cell-free nucleic acids in the sample and the results of the gene expression profiling assay.

In another aspect, provided herein is a kit for detecting and monitoring transplant rejection status of a transplant from a donor in a transplant recipient, wherein the donor and the recipient belong to different species, the kit comprising: (a) one or more PCR reaction oligonucleotide primers and probe sets that hybridize to donor-specific or recipient-specific target sequences in cell-free nucleic acids in a sample from the transplant recipient for PCR quantitation of donor-derived cell-free nucleic acids as absolute copies of donor-derived cell-free nucleic acids in the sample, or as a ratio of donor-derived cell-free nucleic acids to total donor-derived and recipient-derived cell-free nucleic acids in the sample, and (b) instructions for data analysis to determine an amount of donor-derived cell-free nucleic acids. In some embodiments, the PCR quantitation is a qPCR quantitation. In some embodiments, the PCR quantitation is a digital PCR quantitation.

In some embodiments, which may be combined with any of the preceding aspects or embodiments, the donor-derived and/or the recipient derived cell-free nucleic acids are DNA. In some embodiments, which may be combined with any of the preceding aspects or embodiments, the donor-derived and/or the recipient derived cell-free nucleic acids are DNA, RNA, mRNA, miRNA, double-stranded DNA, single-stranded DNA, single-stranded DNA hairpins, DNA/RNA hybrids, RNA hairpins, or a combination thereof. In some embodiments, which may be combined with any of the preceding aspects or embodiments, the amount of donor-derived cell-free nucleic acids is adjusted with a correction factor to correct for differences in genome length between the donor and recipient genomes. In some embodiments, which may be combined with any of the preceding aspects or embodiments, the amount of donor-derived cell-free nucleic acids is adjusted with a correction factor to correct for cell-free nucleic acid fragment size differences between cell-free nucleic acids that are derived from the donor and cell-free nucleic acids that are derived from the recipient. In some embodiments, which may be combined with any of the preceding aspects or embodiments, the amount of donor-derived cell-free nucleic acids is a ratio of donor-derived cell-free nucleic acids to total or recipient-derived cell-free nucleic acids. In some embodiments, which may be combined with any of the preceding aspects or embodiments, the amount of donor-derived cell-free nucleic acids is a percentage of donor-derived cell-free nucleic acids compared to total cell-free nucleic acids. In some embodiments, which may be combined with any of the preceding aspects or embodiments, the transplant is a solid organ, tissue or cell transplant. In some embodiments, which may be combined with any of the preceding aspects or embodiments, the donor of the transplant is an animal. In some embodiments, the animal is a pig.

In another aspect, provided herein is a method for analyzing a biological sample from a transplant recipient who received a solid organ transplant from a donor, wherein the donor and the recipient belong to different species, the method comprising: a) isolating cell-free nucleic acids from a biological sample from the transplant recipient, wherein the cell-free nucleic acids comprise cell-free nucleic acids that are derived from the donor and cell-free nucleic acids that are derived from the recipient; b) generating amplicons by amplifying target regions of the cell-free nucleic acids, wherein the target regions comprise one or more target regions comprising nucleotide sequences that are donor-specific and/or one or more target regions comprising nucleotide sequences that are recipient-specific; c) generating sequence reads from the generated amplicons by sequencing the amplicons, wherein the generated sequence reads comprise one or more sequence reads that correspond to donor-specific genome sequences and/or one or more sequence reads that correspond to recipient-specific genome sequences, and optionally mapping the generated sequence reads to at least donor-specific genome sequences, optionally wherein differences in genome size between donor and recipient are accounted for; and c) quantifying an amount of cell-free nucleic acids from the generated sequence reads in the sample. In some embodiments, the amount of cell-free nucleic acids is an amount of transplant donor-derived cell-free nucleic acids in the sample. In some embodiments, the amount of cell-free nucleic acids is an amount of total cell-free nucleic acids in the sample. In some embodiments, the method further comprises adding one or more quantitative spike-in nucleic acid controls to the sample, and generating sequence reads corresponding the one or more spike-in nucleic acid controls by sequencing the one or more spike-in nucleic acid controls, wherein sequence reads corresponding to the spike-in controls are used to determine an absolute amount of total cell-free nucleic acids in the sample. In some embodiments, the method further comprises adding one or more quantitative spike-in nucleic acid controls to the sample, and generating sequence reads corresponding the one or more spike-in nucleic acid controls by sequencing the one or more spike-in nucleic acid controls, wherein sequence reads from the spike-in controls are used to determine an absolute amount of transplant donor-derived cell-free nucleic acids in the sample. In some embodiments, the one or more quantitative spike-in nucleic acid controls are added before step a). In some embodiments, the one or more quantitative spike-in nucleic acid controls are added after step a). In some embodiments, the one or more quantitative spike-in nucleic acid controls are added before and after step a). In some embodiments, the method further comprises accounting for differences in genome size between donor and recipient using a correction factor to correct for differences in genome length between the donor and recipient genomes.

In another aspect, provided herein is a method for analyzing a biological sample from a transplant recipient who received a solid organ transplant from a donor, wherein the donor and the recipient belong to different species, the method comprising: isolating cell-free nucleic acids from a biological sample from the recipient, wherein the cell-free nucleic acids comprise cell-free nucleic acids that are derived from the donor and cell-free nucleic acids that are derived from the recipient; determining an amount of donor-derived cell-free nucleic acids in the sample by a digital PCR assay, wherein the digital PCR assay comprises: (i) at least one digital PCR assay for one or more single-copy or multi-copy donor-specific targets, or (ii) at least one digital PCR assay for one or more single-copy or multi-copy recipient-specific targets and one or more single-copy or multi-copy donor-specific targets, wherein the number of copies of the donor-specific targets is in reference to a haploid donor genome and the number of copies of the recipient-specific targets is in reference to a haploid recipient genome, and wherein the amount of donor-derived cell-free nucleic acids is determined: (i) as absolute copies of donor-derived cell-free nucleic acids in the sample, or (ii) as a ratio of donor-derived cell-free nucleic acids to total donor-derived and recipient-derived cell-free nucleic acids, by digital PCR quantitation of donor-specific nucleic acids and recipient-specific nucleic acids. In some embodiments, the digital PCR assay comprises (a) a first digital PCR assay for a single-copy or multi-copy recipient-specific target, wherein the number of copies of the recipient-specific target is in reference to a haploid recipient genome, and (b) a second digital PCR assay for a single-copy or multi-copy donor-specific target, wherein the number of copies of the donor-specific target is in reference to a haploid donor genome. In some embodiments, the second digital PCR assay is for a multi-copy donor-specific target. In some embodiments, the digital PCR assay is for (a) at least one single-copy or multi-copy recipient-specific target, wherein the number of copies of the recipient-specific target is in reference to a haploid recipient genome; and (b) at least one single-copy or multi-copy donor-specific target, wherein the number of copies of the donor-specific target is in reference to a haploid donor genome. In some embodiments, the digital PCR assay is for a multi-copy donor-specific target, wherein the number of copies of the donor-specific target is in reference to a haploid donor genome.

### BRIEF DESCRIPTION OF THE DRAWINGS

FIGS. 1A-1B show the accuracy and linearity of digital PCR (dPCR) assays to determine the percentage of pig DNA in samples containing a mixture of pig and human DNA. The results shown in FIG. 1A were generated with singleplex dPCR assays using a two-copy pig-specific target (“Pig assay A”, see Table 2) and a single-copy human-specific target (“Human assay A”, see Table 2) in control samples containing known percentages of control pig and human genomic DNAs. The x-axis shows the known percentage of pig DNA in each sample (i.e., the “Expected % pig DNA”), and the y-axis shows the percentage of pig DNA as measured using the dPCR assay (“Measured % pig DNA”). Each point in the graph represents one sample, and the fit line and corresponding formula on the graph show the linearity of the expected and measured results. The results shown in FIG. 1B were generated with a multiplex dPCR assay comprising two single-copy pig assays (“Pig assay B” and “Pig assay C”, see Table 2) and one single-copy human assay (“Human assay B”, see Table 2).

FIG. 2 provides a longitudinal analysis of percent pig-donor-derived cfDNA (% dd-cfDNA) and copies per ml plasma (cp/ml) in cfDNA samples from a human transplant recipient of a pig heart transplant, assessed using singleplex dPCR (“Human assay A” and “Pig assay A”, see Table 2) to detect a single-copy human-specific target and a two-copy pig-specific target (two pig copies per haploid genome). The percent and cp/ml pig-donor-derived cfDNA (y-axis) were determined in cfDNA samples obtained from the human organ transplant recipient on the days post-transplant, as indicated on the x-axis (i.e., days 6, 13, 19, 25, 33, 46, 55, and 60 after the organ transplantation).

FIG. 3 provides a longitudinal analysis of percent pig-derived cfDNA in cfDNA samples from a human recipient of a pig heart transplant, assessed using shotgun next-generation sequencing (NGS). NGS results were analyzed according to filtering models M1-M7 as described in Example 1 and Table 3, herein. The percent pig-donor-derived cfDNA (y-axis) was determined in cfDNA samples obtained from the human organ transplant recipient on the days post-transplant, as indicated on the x-axis (i.e., days 6, 13, 19, 25, 33, 46, 55, and 60 after the organ transplantation).

FIG. 4 shows a comparison of percent pig-donor-derived cfDNA estimates in samples from a human recipient of a pig heart transplant, as measured by singleplex dPCR (“Human assay A” and “Pig assay A”, see Table 2) or shotgun NGS. The percent pig-donor-derived cfDNA (y-axis) was determined in cfDNA samples obtained from the human organ transplant recipient on the days post-transplantation, as indicated on the x-axis (i.e., days 6, 13, 19, 25, 33, 46, 55, and 60 after the organ transplantation). NGS results were analyzed according to filtering model M7 as described in Example 1 and Table 3, herein.

FIG. 5 shows a comparison of percent pig-donor-derived cfDNA in samples from a human recipient of a pig heart transplant, as measured by singleplex dPCR (“Human assay A” and “Pig assay A”, see Table 2), multiplex dPCR (“Human assay B”, “Pig assay B” and “Pig assay C”, see Table 2), and shotgun NGS. The percent pig-donor-derived cfDNA (y-axis) was determined in cfDNA samples obtained from the human organ transplant recipient on the days post-transplantation, as indicated on the x-axis (i.e., days 33, 46, 49, and 60 after the organ transplantation). NGS results were analyzed according to filtering model M7 as described in Example 1 and Table 3, herein.

FIGS. 6A and 6B show a comparison of percent pig-donor-derived cfDNA (% xcfDNA) in samples from human deceased model recipients (Recipient 1 and Recipient 2) of a pig heart transplant, as measured by multiplex dPCR (“Human assay C” and “Pig assay D”, see Table 2) or shotgun NGS. The percent pig-donor-derived cfDNA (y-axis) was determined in cfDNA samples obtained from the human deceased model recipients post-transplantation, as indicated on the x-axis (i.e., hours 30, 48, 72 after the organ transplantation for Recipient 1, FIG. 6A, and hours 0, 12, 24, 60, 66 for Recipient 2, FIG. 6B). NGS results were analyzed according to filtering model M7 as described in Example 1 and Table 3, herein. FIGS. 6C and 6D show a comparison of pig genomic copies per mL sample from the human deceased model Recipient 1 (FIG. 6C) and Recipient 2 (FIG. 6D) post-transplantation, as measured by multiplex dPCR (“Human assay C” and “Pig assay D”, see Table 2).

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein but are to be accorded the scope consistent with the claims.

### Overview

The present disclosure describes sensitive and non-invasive methods and kits for detecting, predicting, diagnosing and/or monitoring the health status of a transplant and for detecting, predicting, and/or monitoring transplant rejection status in recipients of an organ, cell or tissue transplant from a donor, where the donor and the recipient belong to different species. Such methods and kits are based on the detection and quantitation of donor-specific nucleic acids, such as DNA or RNA, in bodily fluids including, but not limited to, blood, serum, plasma or urine post-transplantation.

Transplantation of organs, cells, or tissues from a donor into a recipient triggers an immune response from the recipient's immune system, which may lead to acute and/or chronic transplant rejection. Without wishing to be bound by theory, it is thought that transplant rejection is associated with the death of cells from the organ, tissue or cell transplant, which results in release of donor-derived nucleic acids such as donor-derived cell-free DNA (dd-cfDNA) from the dying, no longer intact donor cells, into the bloodstream and other bodily fluids of the recipient. Accordingly, the methods of the present disclosure involve analysis of cell-free DNA (cfDNA) in samples from a recipient of an organ, cell or tissue transplant from a donor, where the donor and the recipient belong to different species, to diagnose the status of the transplant based on the amount of dd-cfDNA. As disclosed herein, analysis of cfDNA in samples obtained from a transplant recipient may be carried out using nucleic acid sequencing-based methods (e.g., high-throughput sequencing and/or next-generation sequencing methods), as well as PCR-based methods for quantitating nucleic acids (e.g., quantitative PCR [qPCR] and digital PCR [dPCR]). See, Examples 1 and 2, herein. Advantageously, the nucleic acid sequencing-based methods of the disclosure provide enhanced accuracy in estimates of the amount of dd-cfDNA by accounting for genome size differences between the donor and the recipient. See, Examples 1 and 2, herein. Similarly, the nucleic acid PCR-based methods of the disclosure (e.g., the dPCR-based methods) provide enhanced sensitivity and accuracy in estimates of the rare amount of dd-cfDNA, for example, by detecting donor-specific targets that are present in multiple copies in the haploid genome of the donor. Accordingly, as demonstrated herein, the methods of the disclosure allow for sensitive, accurate and non-invasive monitoring of the status of organ, cell or tissue transplants in a transplant recipient, for example, by monitoring the amounts of dd-cfDNA in bodily fluids (e.g., blood) from the transplant recipient repeatedly and over extended periods of time. See, e.g., Example 2 and Example 3 herein.

### Definitions

As used in the specification and in the appended claims, the singular forms of “a,” “an,” and “the” include plural referents unless the context clearly dictates otherwise.

The term “or” is used herein to mean, and is used interchangeably with, the term “and/or,” unless context clearly indicates otherwise.

The term “sample,” as used herein, refers to any sample obtained from a transplant recipient, such as whole blood, plasma, serum, lymph, peripheral blood mononuclear cells, buccal swabs, saliva, urine, lung lavagae, or tissue from a biopsy.

The term “transplant” as used herein refers to transplants of any cell(s), tissue(s), or organ(s) from a donor into a recipient, including combinations thereof. The term “transplant” with respect to a tissue or organ may refer to a whole tissue or organ (e.g., a whole liver) or portions thereof.

The term “nucleic acid,” as used herein, refers to RNA or DNA that is linear, circular or branched, single or double stranded, or a hybrid thereof. The term also encompasses RNA/DNA hybrids. In some cases, nucleic acid refers to any of DNA, RNA, mRNA, miRNA, double-stranded DNA, single-stranded DNA, single-stranded DNA hairpins, DNA/RNA hybrids, RNA hairpins, and fragments and combinations thereof.

The term “cell-free nucleic acid,” as used herein, refers to nucleic acid(s) present outside of a cell. In some cases, cell-free nucleic acids are nucleic acids that are present outside of a cell, and are present in a bodily fluid (e.g., blood, plasma, serum, urine, etc.) of a transplant recipient. In some embodiments, cell-free nucleic acid(s) refers to any DNA, RNA, mRNA, miRNA, double-stranded DNA, single-stranded DNA, single-stranded DNA hairpins, DNA/RNA hybrids, RNA hairpins, as well as fragments and combinations thereof, present outside of a cell. The term also includes organ-specific or tissue-specific RNA transcripts.

The term “providing a sample,” as used herein, refers to providing a sample, e.g., for use in the methods of the disclosure. In some cases, providing a sample includes performing a process (e.g., performing a physical method) to obtain the sample. In some cases, a sample is provided by the parties or entities performing a method of the disclosure. In other cases, a sample is provided by an entity or party other than the parties or entities performing a method of the disclosure. Thus, in some cases, the methods of the disclosure comprise receiving a sample from another party or source (e.g., a third party that obtained the sample).

The term “generating sequence reads,” as used herein, refers to performing a process to obtain sequence reads, e.g., for use in the methods of the disclosure. For example, generating sequence reads may involve performing a sequencing method (e.g., a Next Generation Sequencing (NGS) method or a high throughput sequencing (HTS) method). Such sequencing methods may involve whole-genome sequencing or targeted sequencing, and may sequence nucleic acids including, but not limited to, genomic DNA, mitochondrial DNA, cDNA obtained from RNA transcripts, or RNA. In some embodiments, at least 500, at least 1000, at least 5000, at least 10000, at least 20000, at least 50000, at least 100000, or more, sequence reads may be generated. Each sequence read may comprise at least 20, at least 50, at least 75, at least 100, at least 150 or more bases per read. The sequence reads may be generated by the parties or entities performing a method of the disclosure. In other cases, sequence reads may be generated by an entity or party other than the parties or entities performing a method of the disclosure. Thus, in some cases, the methods of the disclosure comprise receiving information or knowledge of, or receiving, sequence reads from another party or source (e.g., a third party laboratory that directly generated or acquired the sequence reads).

The term “target sequence,” as used herein, refers to a region of a nucleic acid that comprises a sequence of interest. The term “target,” as used herein, may comprise one or more target sequences.

All methods described herein can be performed in any suitable order unless otherwise indicated herein or otherwise clearly contradicted by context. The use of any and all examples, or exemplary language (e.g., “such as”) provided with respect to certain embodiments herein is intended merely to illustrate the embodiments, and does not pose a limitation on the scope of the embodiments otherwise claimed. No language in the specification should be construed as indicating any non-claimed element essential to the practice of the embodiments.

### I. Methods of the Disclosure

Certain aspects of the present disclosure relate to methods for detecting, predicting, diagnosing and/or monitoring transplant rejection status of an organ, tissue or cell transplant from a donor in a transplant recipient; methods for treating transplant rejection of an organ, tissue or cell transplant from a donor in a transplant recipient; and methods for adjusting immunosuppressive therapy in a transplant recipient of an organ, tissue or cell transplant from a donor. Other aspects of the disclosure relate to methods of analyzing a biological sample from a transplant recipient, for example, to quantify cell-free nucleic acids, such as donor-derived, recipient-derived, and/or total cell-free nucleic acids (e.g., using sequencing or a PCR-based method). In some embodiments, an organ, tissue or cell transplant according to the present disclosure is a cross-species transplant (i.e., a xenogeneic or heterologous transplant), wherein the organ, tissue or cell transplant donor is of a different species from the transplant recipient. Accordingly, in some embodiments, the methods of the disclosure comprise detecting, predicting, diagnosing and/or monitoring transplant rejection status of a cross-species, xenogeneic or heterologous organ, tissue or cell transplant in a transplant recipient; treating transplant rejection of a cross-species, xenogeneic or heterologous organ, tissue or cell transplant in a transplant recipient; and/or adjusting immunosuppressive therapy in a transplant recipient of a cross-species or xenogeneic organ, tissue or cell transplant.

In some embodiments, the methods of the disclosure comprise providing a sample from a recipient of an organ, tissue or cell transplant from a donor after the transplantation, wherein the sample from the transplant recipient comprises cell-free nucleic acids (e.g., cfDNA and/or cfRNA) that are derived from the donor and cell-free nucleic acids (e.g., cfDNA and/or cfRNA) that are derived from the recipient; determining an amount of donor-derived cell-free nucleic acids in the cell-free nucleic acids from the sample; and detecting transplant rejection based on the determined amount of donor-derived cell-free nucleic acids. In some embodiments, the methods comprise detecting transplant rejection if the determined amount of donor-derived cell-free nucleic acids exceeds a predetermined threshold. In some embodiments, the methods of the disclosure comprise administering an immunosuppressant treatment to the transplant recipient based, at least in part, on the detection of transplant rejection (e.g., based on the determined amount of donor-derived cell-free nucleic acids). In some embodiments, the methods of the disclosure comprise adjusting immunosuppressant treatment of the transplant recipient based, at least in part, on the detection of transplant rejection (e.g., based on the determined amount of donor-derived cell-free nucleic acids).

In some embodiments, the amount of donor-derived cell-free nucleic acids is determined using sequencing based-methods, such as high-throughput sequencing (HTS) and/or next-generation sequencing (NGS), as described in greater detail below. In some embodiments, the amount of donor-derived cell-free nucleic acids is determined using nucleic acid PCR-based quantitation methods, such as quantitative PCR (qPCR) or digital PCR (dPCR), as described in greater detail below.

A. Quantitating Cell-Free Nucleic Acids

Certain aspects of the present disclosure relate to methods for determining an amount of donor-derived cell-free nucleic acids in cell-free nucleic acids from a sample from a transplant recipient using sequencing-based methods or nucleic acid PCR-based quantitation methods, as described in greater detail below.

Other aspects of the disclosure relate to methods for quantifying cell-free nucleic acids (such as recipient-derived, donor-derived and/or total cell-free nucleic acids) in a biological sample from a transplant recipient.

Sequencing-Based Methods

In some embodiments, the methods of the disclosure comprise determining an amount of cell-free nucleic acids (such as recipient-derived, donor-derived, and/or total cell-free nucleic acids) in cell-free nucleic acids from a sample from a transplant recipient using a nucleic acid sequencing-based method.

Various methods and protocols for nucleic acid sequencing and analysis that are well-known in the art may be used in the methods of the disclosure. For example, DNA sequencing may be accomplished using high-throughput sequencing (HTS) techniques, whole genome sequencing, shotgun sequencing, whole exome sequencing, targeted sequencing, Sanger sequencing, a massively parallel sequencing technique, or next-generation sequencing (NGS). Examples of next generation and high-throughput sequencing include, for example, massively parallel signature sequencing, polony sequencing, 454 pyrosequencing, Illumina (Solexa) sequencing with HiSeq, MiSeq, and other Illumina platforms, SOLiD sequencing, ion semiconductor sequencing (Ion Torrent), nanopore sequencing (see, e.g., the website: nanoporetech.com/applications/short-fragment-mode), DNA nanoball sequencing, heliscope single molecule sequencing, single molecule real time (SMRT) sequencing, MassARRAY®, and Digital Analysis of Selected Regions (DANSR™). See, e.g., Stein R A (2008). “Next-Generation Sequencing Update”. Genetic Engineering & Biotechnology News 28 (15); Quail et al., (2012). “A tale of three next generation sequencing platforms: comparison of Ion torrent, pacific biosciences and illumina MiSeq sequencers”. BMC Genomics 13 (1): 341; Liu et al., (2012). “Comparison of Next-Generation Sequencing Systems”. Journal of Biomedicine and Biotechnology 2012: 1-11; Qualitative and quantitative genotyping using single base primer extension coupled with matrix-assisted laser desorption/ionization time-of-flight mass spectrometry (MassARRAY®). Methods Mol Biol. 2009; 578:307-43; Chu et al., A novel approach toward the challenge of accurately quantifying fetal DNA in maternal plasma. Prenat Diagn 2010; 30: 1226-9; and Suzuki et al., Characterization of circulating DNA in healthy human plasma. Clinica chimica acta; international journal of clinical chemistry 2008; 387:55-8.

In some embodiments, the methods of the disclosure comprise generating sequence reads corresponding to one or more donor-specific genome sequences and/or recipient-specific genome sequences in cell-free nucleic acids from a sample obtained from a transplant recipient. In some embodiments, the sequence reads are generated using any suitable sequencing method known in art, such as high-throughput sequencing (HTS) techniques or next-generation sequencing (NGS), e.g., as described above. In some embodiments, the sequence reads are generated using NGS shotgun sequencing, for example, using a MiSeq or NextSeq sequencing platform from Illumina. In some embodiments, the sequence reads are generated using paired-end sequencing. In some embodiments, the sequence reads are generated using amplicon-based NGS, wherein target regions of the genome are amplified, e.g., using PCR, prior to sequencing. In some embodiments, the sequence reads are generated using non-amplicon-based NGS, e.g., using hybrid-capture NGS. In some embodiments, the sequence reads are grouped into regions of selected sizes, of the same size, for the donor and recipient genomes. In some embodiments, the methods comprise mapping the sequence reads to donor- and/or recipient-specific genome sequences. In some embodiments, the mapping comprises aligning the sequence reads to a reference sequence. The aligning or mapping may be carried out using any suitable method known in the art, such as using a Burrows-Wheeler Aligner (BWA). In some embodiments, sequence read alignment is carried out with a minimum alignment score cutoff of about 90%, allowed percent gaps of about 5%, and/or no split alignments. In some embodiments, the reference sequence comprises a reference genome corresponding to the donor, and/or a reference genome corresponding to the recipient. In some embodiments, the reference sequence is a combined reference sequence comprising a reference genome corresponding to the donor and a reference genome corresponding to the recipient. In some embodiments, the methods comprise excluding from the aligned or mapped sequence reads one or more sequence reads with multiple matches to the reference sequence. In some embodiments, the methods comprise excluding from the aligned or mapped sequence reads one or more sequence reads with missing mates, translocated sequence reads, and sequence reads with improper orientation. In some embodiments, the methods comprise excluding from the aligned or mapped sequence reads one or more sequence reads with an alignment score of less than 95% or less than 98%. In some embodiments, the methods comprise excluding from the aligned or mapped sequence reads one or more partially aligned sequence reads or soft-clipped sequence reads. In some embodiments, the methods comprise excluding from the aligned or mapped sequence reads one or more partially aligned sequence reads or hard and soft-clipped sequence reads, or where alignments with MAPQ are smaller than 10.

In some embodiments, the amount of donor-derived cell-free nucleic acids in cell-free nucleic acids from a sample from a transplant recipient is determined based on the percent of the sequence reads that align to the donor genome as a fraction of the sequence reads that align to the donor and recipient genomes.

In some embodiments, the methods of the disclosure comprise accounting for genome size differences between the recipient and donor to determine the amount of donor-derived cell-free nucleic acids in the sample.

In some embodiments, genome size differences between the recipient and donor are accounted for based on sequencing coverage of the donor and recipient genomes in the generated sequence reads. In some embodiments, genome size differences between the recipient and donor are accounted for based on average coverage across one or more regions of selected sizes of the donor and recipient genomes in the generated sequence reads. In some of such embodiments, the amount of donor-derived cell-free nucleic acids is determined as a percentage of average coverage across one or more regions of selected sizes of the donor and recipient genomes. In some embodiments, the one or more regions of selected sizes are large enough to have sufficient coverage. In some embodiments, background noise is low enough to differentiate donor and recipient genomes. In some cases, the amount of donor-derived cell-free nucleic acids is determined as m_{d}/(m_{d}+mᵣ)×100, wherein m_{d} is the median of average coverage across one or more regions of selected sizes of the donor genome, and mᵣ is the median of average coverage across one or more regions of selected sizes of the recipient genome. In some embodiments, the regions of selected sizes of the donor and recipient genomes comprise any of up to 1 megabases (Mb), between about 1 Mb and about 10 Mb, up to 10 Mb, between about 10 Mb and about 100 Mb, or up to 100 Mb. In some embodiments, the regions of selected sizes of the donor and recipient genomes comprise up to 1M bases, up to 10M bases, or up to 100M bases.

In some embodiments, genome size differences between the recipient and donor are accounted for using a correction factor. For example, for donor genomes that are smaller than the recipient genome, the correction factor may be obtained by multiplying the fraction of the sequence reads that are mapped to the donor-specific genome sequences by the ratio of the donor genome size to the recipient genome size.

The sizes of donor-derived and recipient-derived cell-free nucleic acid fragments may differ, see, e.g., Example 2 and Table 4 herein, which show that differences were observed in the mean fragment size of pig-donor-derived cell-free DNA fragments as compared to cell-free DNA fragments from a human transplant recipient. Such differences in fragment sizes between donor-derived and recipient-derived cell-free nucleic acids could result in over-estimation or under-estimation of the amount of donor-derived cell-free nucleic acids. Accordingly, in some embodiments, the methods comprise accounting for possible over-estimation or under-estimation of the amount of donor-derived cell-free nucleic acids due to differences in the fragment sizes of donor-derived and recipient-derived cell-free nucleic acids, e.g., using a correction factor. For example, the correction factor may be determined by fragment size comparison studies in contrived and in-silico samples. In addition, differences in fragment sizes between donor-derived and recipient-derived cell-free nucleic acids could also result in over-estimation or under-estimation of sequencing coverage of the donor and recipient genomes in the generated sequence reads. For example, larger coverage may be obtained with smaller nucleic acid fragments in comparison to larger nucleic acid fragments. Accordingly, in some embodiments, the methods comprise accounting for possible over-estimation or under-estimation of sequencing coverage of the donor and recipient genomes due to fragment size differences between donor-derived and recipient-derived cell-free nucleic acids, e.g., using a correction factor. For example, the correction factor may be determined by multiplying a coverage fraction (e.g., a fraction of average coverage across regions of selected sizes of the donor and recipient genomes) by the ratio of the mean donor nucleic acid fragment size to the mean recipient nucleic acid fragment size.

In some embodiments, absolute quantitation of the amount of donor-derived cell-free nucleic acids in cell-free nucleic acids from a sample from a transplant recipient may be accomplished by inclusion in the sample from the transplant recipient, or in cell-free nucleic acids from the transplant recipient, of control nucleic acids of known concentrations or amounts prior to generating sequence reads (e.g., a quantitative spike-in nucleic acid control). The absolute amount of donor-derived cell-free nucleic acids is then determined based on the sequence reads corresponding to the control nucleic acids and to the donor-derived cell-free nucleic acids.

Where there are multiple cell-free nucleic acid samples from a transplant recipient to be sequenced, such as when multiple samples are taken from the transplant recipient over time or when samples are taken from various transplant recipients, each sample may be sequenced individually, or multiple samples may be sequenced together using multiplex sequencing.

Nucleic Acid PCR-Based Quantitation-Based Methods

In some embodiments, the methods of the disclosure comprise determining an amount of cell-free nucleic acids (such as recipient-derived, donor-derived, and/or total cell-free nucleic acids) in cell-free nucleic acids from a sample obtained from a transplant recipient using a nucleic acid PCR quantitation method.

Various methods and protocols for nucleic acid quantitation are well-known in the art, such as quantitative nucleic acid amplification methods, including quantitative polymerase chain reaction (qPCR), digital PCR (dPCR), and real time dPCR.

qPCR or real-time quantitative PCR refer to a method of using PCR to amplify and quantify target nucleic acids at the same time. As is known in the art, quantitation of amplified nucleic acids accumulated in a qPCR reaction is performed in real time after every amplification cycle. The absolute quantitation may be performed using a standard curve, and the quantitation may be performed using detectably labeled probes, primers or dyes, such as: intercalating dyes that bind to double stranded DNA products, e.g., YO-PRO-I, SYBR green, and the like (see, e.g., Ishiguro et al. Anal. Biochem., 229: 207-213 (1995); Tseng et al Anal. Biochem., 245: 207-212 (1997); Morrison et al. Biotechniques, 24: 954-962 (1998)); primers having hairpin structures with a fluorescent molecule held in proximity to a fluorescent quencher until forced apart by primer extension (see, e.g. Whitecombe et al. Nature Biotechnology, 17: 804-807 (1999; “AMPLIFLUOR primers”); or sequence-specific probes, usually comprising a fluorescent molecule in proximity to a fluorescent quencher until an oligonucleotide moiety to which they are attached specifically binds to an amplification product (see, e.g. Gelfand et al U.S. Pat. No. 5,210,015 (TAQMAN); Nazarenko et al. Nucleic Acids Research, 25: 2516-2521 (1997) (scorpion probes); Tyagi et al. Nature Biotechnology, 16:49-53 (1998) (molecular beacons)). Other suitable dyes, primers or probes that may be used include dual hybridization probes, eclipse probes, LUX PCR primers, and QZyme PCR primers. Such dyes, primers or probes may be used in connection with qPCR as described herein, or they may be used to measure the total amount of reaction product at the completion of a reaction. In some embodiments, the detectably labeled probes, primers or dyes are fluorescently labeled, e.g., using one or more of FAM, HEX, ATTO 550, ROX, Cy5, Cy5.5, or any other suitable dye.

Digital PCR or dPCR refers to a method that allows for absolute quantitation of target nucleic acids As is known in the art, dPCR is performed by partitioning a PCR reaction into a multitude of partitions such that each partition contains one, a few or no target sequences. After the PCR reaction, i.e., at the endpoint of the PCR reaction, amplification-positive and total number of partitions are used to quantitate the target sequence using Poisson statistics. Positive partitions may be identified using detectably labeled probes, primers or dyes, e.g., as described above for qPCR. In some embodiments, the detectably labeled probes, primers or dyes are fluorescently labeled, e.g., using one or more of FAM, HEX, ATTO 550, ROX, Cy5, Cy5.5, or any other suitable dye. For a description of dPCR methods, see, e.g., Hindson et al. (2011) Anal. Chem. 83(22):8604-8610; Pohl and Shih (2004) Expert Rev. Mol. Diagn. 4(1):41-47; Pekin et al. (2011) Lab Chip 11 (13): 2156-2166; Pinheiro et al. (2012) Anal. Chem. 84 (2): 1003-1011; Day et al. (2013) Methods 59(1):101-107; and Quan et al., Sensors (Basel). 2018 April; 18(4): 1271. The multitude of partitions may be generated using any suitable method known in the art, such as by partitioning the PCR reaction into micro-wells, chambers or droplets. In instances where the multitude of partitions are generated by partitioning the PCR reaction into droplets, the dPCR method may be referred to as droplet digital PCR, e.g., as available from BioRad (ddPCR™) or Stilla (e.g., Crystal digital PCR™)

In some embodiments, the methods of the disclosure comprise determining an amount of donor-derived cell-free nucleic acids in a sample using a digital PCR assay.

In some embodiments, the dPCR assay comprises one or more singleplex dPCR assays. In some of such embodiments, the singleplex dPCR assays may comprise one or more recipient-specific singleplex dPCR assays, and/or one or more donor-specific singleplex dPCR assays. In some embodiments, the recipient-specific singleplex PCR assay(s) and donor-specific singleplex PCR assay(s) each have a single-copy target per haploid genome. In some embodiments, some or all of the recipient-specific singleplex dPCR assay(s) and/or donor-specific singleplex dPCR assay(s) have a multi-copy (e.g., 2- or more copy) target per haploid genome, that generates more target-positive partitions for the same amount of sample input (e.g., two or multiple times more target-positive partitions for the same amount of sample input) to increase assay detection and quantitation sensitivities and precision. This may be particularly helpful in cases where donor derived cell-free nucleic acids are in rare amount in the total cell-free nucleic acids from the sample. In some embodiments, the multi-copy target PCR assay may target multiple members of a multi-gene family such as the actin, tubulin, rRNA gene families, or multiple members of repetitive sequences such as the Alu sequences, or the multiple copies of the mitochondrial DNAs per diploid cell. In some embodiments, the dPCR assay comprises at least a singleplex digital PCR assay for a single-copy or multi-copy donor-specific target, wherein the number of copies of the donor-specific target is in reference to a haploid donor genome; and/or at least a singleplex digital PCR assay for a single-copy or multi-copy recipient-specific target, wherein the number of copies of the recipient-specific target is in reference to a haploid recipient genome. In some embodiments, the digital PCR assay comprises a first singleplex digital PCR assay and a second singleplex digital PCR assay, wherein: (a) the first singleplex digital PCR assay is for a single-copy or multi-copy recipient-specific target, wherein the number of copies of the recipient-specific target is in reference to a haploid recipient genome, and (b) the second singleplex digital PCR assay is for a single-copy or multi-copy donor-specific target, wherein the number of copies of the donor-specific target is in reference to a haploid donor genome. In some embodiments, the second singleplex digital PCR assay is for a multi-copy donor-specific target. The quantitation of cell-free nucleic acids by different dPCR assays targeting different copy-number targets can be normalized to genome copy quantitation for analysis of relative quantitation of donor-derived genomes to total donor and recipient genomes, or absolute quantitation of donor-derived genome copies per unit of sample, e.g. copies/ml of plasma sample.

In some embodiments, the dPCR assay is a multiplex dPCR assay comprising two or more PCR assays in a single dPCR reaction. In some of such embodiments, the multiplex dPCR assay may comprise at least one donor-specific PCR assay. In some embodiments, the multiplex dPCR assay may comprise at least one donor-specific PCR assay and at least one recipient-specific PCR assay for quantitating donor-derived cell-free nucleic acids relative to recipient or total cell-free nucleic acids. In some embodiments, the multiplex dPCR assay may comprise recipient-specific PCR assays and donor-specific PCR assays, each targeting at least one single-copy target per haploid genome of the species. In some embodiments, the multiplex dPCR assay may comprise recipient-specific PCR assay(s) and donor-specific PCR assay(s), each targeting at least one multi-copy target per haploid genome to improve target detection and quantitation sensitivities with limited sample input. In some embodiments, the multiplex dPCR assay may comprise at least one donor-specific PCR assay with at least one single-copy or multi-copy target (e.g., a 2- or more copy target) corresponding to the haploid donor genome (i.e., each donor-specific PCR assay detects at least one single-copy or multi-copy target corresponding to the donor), wherein the number of copies of the target are in reference to the haploid genome of the donor. In some embodiments, the multiplex dPCR assay may comprise one or more, e.g., between one and five, donor-specific PCR assays with at least one single-copy or multi-copy target (e.g., at least one two- or more copy target) corresponding to the donor genome (i.e., each donor-specific PCR assay detects at least one single-copy or multi-copy target corresponding to the donor), wherein the number of copies of the target are in reference to the haploid genome of the donor. In some embodiments, the multiplex dPCR assay may comprise at least one recipient-specific PCR assay with at least one single-copy or multi-copy target corresponding to the haploid recipient genome (i.e., each recipient-specific PCR assay detects at least one single-copy or multi-copy target corresponding to the recipient), wherein the number of copies of the target are in reference to the haploid genome of the recipient. In some embodiments, the multiplex dPCR assay may comprise one or more, e.g., between one and five, recipient-specific PCR assays with at least one single-copy or multi-copy target corresponding to the recipient genome (i.e., each recipient-specific PCR assay detects at least one single-copy or multi-copy target corresponding to the recipient), wherein the number of copies of the target are in reference to the haploid genome of the recipient. In some embodiments, the digital PCR assay comprises at least a multiplex digital PCR assay for two or more single-copy or multi-copy donor-specific targets and/or recipient-specific targets in a single digital PCR reaction, wherein the number of copies of the donor-specific targets is in reference to a haploid donor genome and the number of copies of the recipient-specific targets is in reference to a haploid recipient genome. In some embodiments, the multiplex digital PCR assay is for: (a) at least one single-copy or multi-copy recipient-specific target, wherein the number of copies of the recipient-specific target is in reference to a haploid recipient genome, and (b) at least one single-copy or multi-copy donor-specific target, wherein the number of copies of the donor-specific target is in reference to a haploid donor genome. In some embodiments, the multiplex digital PCR assay is for at least one multi-copy donor-specific target, wherein the number of copies of the donor specific-target is in reference to a haploid donor genome.

In some embodiments, the dPCR assay comprises at least one digital PCR assay for one or more single-copy or multi-copy donor-specific targets. In some embodiments, the dPCR assay comprises at least one digital PCR assay for one or more single-copy or multi-copy recipient-specific targets and one or more single-copy or multi-copy donor-specific targets. In some embodiments, the dPCR assay comprises a first digital PCR assay for a single-copy or multi-copy recipient-specific target, wherein the number of copies of the recipient-specific target is in reference to a haploid recipient genome, and a second digital PCR assay for a single-copy or multi-copy donor-specific target, wherein the number of copies of the donor-specific target is in reference to a haploid donor genome. In some embodiments, the second digital PCR assay is for a multi-copy donor-specific target.

In some embodiments, the digital PCR assay is for at least one single-copy or multi-copy recipient-specific target, wherein the number of copies of the recipient-specific target is in reference to a haploid recipient genome; and at least one single-copy or multi-copy donor-specific target, wherein the number of copies of the donor-specific target is in reference to a haploid donor genome. In some embodiments, the digital PCR assay is for a multi-copy donor-specific target, wherein the number of copies of the donor-specific target is in reference to a haploid donor genome.

In some embodiments, a single-copy target corresponding to the recipient is present as a single copy in the haploid genome of the recipient, and is not present in the genome of the donor. In some embodiments, a single-copy target corresponding to the donor is present as a single copy in the haploid genome of the donor, and is not present in the genome of the recipient. In some embodiments, a multi-copy target (e.g., a two- or more copy target) corresponding to the donor is present in multiple (e.g., two or more) copies in the haploid genome of the donor, and is not present in the genome of the recipient. In some embodiments, the target corresponding to the donor and/or the target corresponding to the recipient are in intronic regions of the donor or recipient genomes. In some embodiments, the target corresponding to the donor and/or the target corresponding to the recipient are in mitochondrial DNA of the donor or recipient genomes. In some embodiments, the multi-copy (e.g., two- or more-copy) target corresponding to the donor comprises a sequence in a multigene family in the genome of the donor. Non-limiting examples of multigene families include rRNA, Hox, Histone, and tubulin gene families.

In some embodiments, each recipient-specific PCR assay comprises a primer pair that detects a recipient-specific target (e.g., a single-copy or multi-copy target corresponding to the recipient), for example, by direct amplification of the recipient-specific target during the PCR reaction. In some embodiments, each donor-specific PCR assay comprises a primer pair that detects a donor-specific target (e.g., a single-copy or a multi-copy target corresponding to the donor), for example, by direct amplification of the donor-specific target during the PCR reaction. In some embodiments, each primer in a primer pair is between about 5 and about 50, between about 10 and about 50, between about 15 and about 30, between about 15 and about 25, between about 18 and about 30, or between about 20 and about 30 bases in length, including any value within each of the recited ranges. In some embodiments, each primer in a primer pair comprises a melting temperature (Tₘ) of any of about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., or about 70° C. In some embodiments, each primer in a primer pair comprises a melting temperature (Tₘ) of about 60° C.

As described above, detection of donor-specific- or recipient-specific-positive partitions in a dPCR assay may be performed using any suitable detection reagent, such as using DNA binding fluorescence dyes (such as SYBR green or EvaGreen) or detectably-labeled probes (e.g., fluorescently-labeled probes). In cases where donor-specific- or recipient-specific-positive partitions are detected using detectably-labeled probes, the recipient-specific PCR or donor-specific PCR assay comprises detectably-labeled probes suitable for detecting the recipient-specific or donor-specific targets separately. In some of such embodiments, each recipient-specific PCR assay comprises a primer pair that detects a recipient-specific target (e.g., a single-copy or multi-copy target corresponding to the recipient), and a detectably-labeled probe that hybridizes to the recipient-specific target; and/or each donor-specific PCR assay comprises a primer pair that detects a donor-specific target (e.g., a single-copy or a multi-copy target corresponding to the donor), and a detectably-labeled probe that hybridizes to the donor-specific target. In some embodiments, the detectably-labeled probe comprises an oligonucleotide configured to hybridize to the recipient-specific target or to the donor-specific target, a fluorescent label, and a quencher. In some embodiments, the fluorescent label is on one end of the oligonucleotide and the quencher is on the other end of the oligonucleotide. In some embodiments, the fluorescent label is any label or dye known in the art, including, but not limited to, FAM, HEX, ATTO 550, ROX, Cy5, and Cy5.5. In some embodiments, the quencher is any quencher known in the art, e.g., BHQ, IABkFQ, TAMRA. In some embodiments, the oligonucleotide comprises a sequence of between about 5 and about 50, between about 10 and about 50, between about 15 and about 30, between about 15 and about 25, or between about 20 and about 30 bases in length, including any value within each of the recited ranges. In some embodiments, the probe comprises a melting temperature (Tₘ) of any of about 60° C., about 61° C., about 62° C., about 63° C., about 64° C., about 65° C., about 66° C., about 67° C., about 68° C., about 69° C., or about 70° C. In some embodiments, the probe comprises a melting temperature (Tₘ) of between about 65° C. and about 70° C., including any value within this range.

In some embodiments, the dPCR assay comprises one or more singleplex PCR assays (e.g., a recipient-specific PCR assay, and/or a donor-specific PCR assay), and each singleplex PCR assay comprises use of a detection reagent with a unique dye or detectable label (e.g., a unique fluorescent dye or label). For example, in a dPCR assay comprising one or more single PCR assays (e.g., a recipient-specific PCR assay, and/or a donor-specific PCR assay), the recipient-specific PCR assay comprises a detection reagent with a first fluorescent label, and the donor-specific PCR assay comprises a detection reagent with a second fluorescent label that is different from the first fluorescent label. Thus, in one embodiment, the dPCR assay comprises a recipient-specific PCR assay comprising a primer pair that detects a recipient-specific target (e.g., a single-copy or multi-copy target corresponding to the recipient), and a detectably-labeled probe that hybridizes to the recipient-specific target, wherein the detectably-labeled probe is labeled with a first fluorescent label; and a donor-specific PCR assay comprising a primer pair that detects a donor-specific target (e.g., a single-copy or a multi-copy target corresponding to the donor), and a detectably-labeled probe that hybridizes to the donor-specific target, wherein the detectably-labeled probe is labeled with a second fluorescent label that is different from the first fluorescent label.

In some embodiments, the dPCR assay is a multiplex dPCR assay comprising two or more PCR assays in a single dPCR reaction, e.g., at least one donor-specific PCR assay, for donor-derived cell-free nucleic acid quantitation as absolute copies of donor-derived cell-free nucleic acids in sample, for example, as absolute donor (genomic) copies per mL sample, e.g., copies per mL plasma, or as donor (genomic) copies per amount DNA, e.g., copies per ng DNA. In some embodiments, the dPCR assay is a multiplex dPCR assay comprising two or more PCR assays in a single dPCR reaction, e.g., at least one donor-specific PCR assay, and one or more recipient-specific PCR assays for relative donor-derived cell-free nucleic acid quantitation in reference to recipient derived cell-free nucleic acids or total donor- and recipient-derived cell-free nucleic acids in the sample, wherein a detection reagent comprising a unique dye or detectable label (e.g., a unique fluorescent dye or label) is used for each PCR assay in the multiplex dPCR reaction; or wherein at least two PCR assays in the multiplex dPCR reaction comprise a detection reagent comprising the same dye or detectable label (e.g., the same fluorescent dye or label). Thus, in one embodiment, in a multiplex dPCR assay comprising at least one recipient-specific PCR assay and at least one donor-specific PCR assay in a single dPCR reaction, each PCR assay in the dPCR reaction may comprise a detection reagent with a unique fluorescent dye or label. For example, a multiplex dPCR assay may comprise at least one recipient-specific PCR assay with a single-copy or multi-copy target corresponding to the recipient and at least one donor-specific PCR assay with a single-copy or a multi-copy (e.g., 2- or more copy) target corresponding to the donor, wherein each PCR assay in the dPCR reaction (i.e., each recipient-specific PCR assay and each donor-specific PCR assay) comprises a detection reagent with a unique fluorescent dye or label. In another embodiment, in a multiplex dPCR assay comprising at least one recipient-specific PCR assay and at least one donor-specific PCR assay in a single dPCR reaction, at least two recipient-specific or donor-specific PCR assays in the dPCR reaction comprise a detection reagent comprising the same dye or detectable label (e.g., the same fluorescent dye or label). For example, a multiplex dPCR assay may comprise at least one recipient-specific PCR assay with a single-copy or multi-copy target corresponding to the recipient and two or more donor-specific PCR assays with a single-copy or a multi-copy (e.g., 2- or more copy) target corresponding to the donor, wherein at least two donor-specific PCR assays in the dPCR reaction comprise a detection reagent with the same fluorescent dye or label.

The amount of donor-derived cell-free nucleic acids present in a cell-free nucleic acid sample from a transplant recipient (e.g., as determined according to the methods of the disclosure) may be expressed in a variety of ways. In some embodiments, the amount of donor-derived cell-free nucleic acids is determined as a percentage of the total cell-free nucleic acids in the sample. In some embodiments, the amount of donor-derived cell-free nucleic acids is determined as absolute copies of donor-derived cell-free nucleic acids in the sample. In some embodiments, the amount of donor-derived cell-free nucleic acids is determined as a ratio of donor-derived cell-free nucleic acids to the total cell-free nucleic acids (e.g., total donor-derived and recipient-derived cell-free nucleic acids) in the sample. In some embodiments, the amount of donor-derived cell-free nucleic acids is determined as a ratio of donor-derived cell-free nucleic acids to recipient-derived cell-free nucleic acids in the sample. In some embodiments, the amount of donor-derived cell-free nucleic acids is determined as a ratio or percentage compared to one or more reference nucleic acids molecules in the sample. For example, the amount of donor-derived cell-free nucleic acids can be determined to be 10% of the total nucleic acid (or total DNA or RNA) molecules in the cell-free nucleic acids sample. Alternatively, the amount of donor-derived cell-free nucleic acids can be a ratio of 1:10 compared to the total nucleic acid (or DNA or RNA) molecules in the cell-free DNA sample. In some embodiments, the amount of donor-derived cell-free nucleic acids can be determined as a concentration. For example, the amount of donor-derived cell-free nucleic acids in the cell-free nucleic acid sample can be determined to be 1 μg per mL sample; 100 donor (genomic) copies per mL sample; 10 donor (genomic) copies per amount DNA, such as 10 donor (genomic) copies per ng DNA, etc. The values described here are merely exemplary to illustrate various ways to express quantities of donor-derived cell-free nucleic acids. The percentage of donor-derived cell-free nucleic acids in the cell-free nucleic acid sample from a transplant recipient may be extremely low. It is noted that the quantity of recipient-derived cell-free nucleic acids in the cell-free nucleic acid sample may also be expressed in the manners as described for donor-derived cell-free nucleic acid. Additional methods of expressing the quantity of a given source, type, or sequence of nucleic acids in a cell-free nucleic acid sample will be readily apparent to one of skill in the art.

In some embodiments, the amount of donor-derived cell-free nucleic acids is determined in reference to the amount of sample analyzed by dPCR assay. In some embodiments, the amount of donor-derived cell-free nucleic acids may be determined as the number of copies of donor genomes in the sample from the transplant recipient (e.g., the copies of donor genomes per mL of sample, or per amount of cell-free nucleic acids in the sample). In one embodiment, the amount of donor-derived cell-free nucleic acids may be determined as the number of copies of donor genomes per volume of sample from the recipient, such as the number of copies of donor genomes per volume (e.g., mL) of plasma from the recipient. In another embodiment, the amount of donor-derived cell-free nucleic acids may be determined as the number of copies of donor genomes per amount of total cell-free nucleic acids in the sample from the recipient, such as the number of copies of donor genomes per ng of cell-free nucleic acids in the sample from the recipient. In one example, the number of copies of donor-specific target sequence molecules as determined by dPCR assay (e.g., as a number of copies of donor DNA molecules per μL) is adjusted to the number of copies of donor-specific haploid genomes in the dPCR assay (e.g., in a donor-specific PCR assay for a two-copy target corresponding to the donor haploid genome, the number of copies of donor target sequences as determined by dPCR assay is divided by the two copies/haploid genome). The number of copies of recipient target sequence molecules as determined by dPCR assay may be adjusted as described above for donor-specific target sequences. Next, the number of copies of donor genomes in the sample from the transplant recipient (e.g., the copies of donor genomes per mL of sample, or per amount of cell-free nucleic acids in the sample) is determined based on the volume used for the dPCR assay, the volume of cell-free nucleic acids from the sample (e.g., the volume of cell-free nucleic acids extracted from a sample from the transplant recipient), and the volume of the sample from the transplant recipient (e.g., the volume of the sample from the transplant recipient from which the cell-free nucleic acids were extracted). In cases where two or more PCR assays in the dPCR reaction (e.g., in a multiplex dPCR assay) comprise a detection reagent with the same fluorescent dye or label, the method further comprises normalizing to the number of PCR assays comprising a detection reagent with the same fluorescent dye or label. In one example of a multiplex dPCR assay with two donor-specific single-copy target PCR assays in the dPCR reaction comprising a detection reagent with the same fluorescent dye or label, the number of copies of donor-specific target sequences as determined by dPCR assays (e.g., as a sum of number of copies of donor-specific sequences per μL) is adjusted (divided) by the number of donor-specific PCR assays in the dPCR reaction comprising a detection reagent with the same fluorescent dye or label (e.g., in the dPCR assay with two donor-specific PCR assays comprising a detection reagent with the same fluorescent dye or label, the number of copies of donor genomes per μL is obtained by dividing the measured total copies/μL by two), and to the number of copies of each donor-specific target in the PCR assay (e.g., in a donor-specific PCR assay for a two-copy target corresponding to the donor, the number of copies of donor genomes is obtained by dividing the measured copies/μL by two). The number of copies of recipient genomes as determined by dPCR assay (e.g., as a number of copies of recipient genomes per μL) may also be adjusted as described above for the number of copies of the donor genome. Next, the number of copies of donor genomes in the sample from the transplant recipient (e.g., the copies of donor genomes per mL of sample, or per amount of cell-free nucleic acids in the sample) is determined based on the volume used for the dPCR assay, the volume of cell-free nucleic acids from the sample (e.g., the volume of cell-free nucleic acids extracted from a sample from the transplant recipient), and the volume of the sample from the transplant recipient (e.g., the volume of the sample from the transplant recipient from which the cell-free nucleic acids were extracted). In some embodiments, the amount of donor-derived cell-free nucleic acids is determined using one or more, or all of the steps of: (1) dividing donor target copies/μl from dPCR instrument by the number of donor target copies per haploid genome (if more than 1 donor target assay is combined in a fluorescent channel, also divide by the number of donor target assays in the fluorescent channel), yielding donor target copies/μl in the reaction; (2) calculating total donor genome copies in the reaction by multiplying donor target copies/μl in the reaction by volume of reaction; (3) dividing the total donor genome copies in the reaction by the volume of nucleic acid sample used in the reaction, yielding total donor genome copies per volume of nucleic acid sample; (4) multiplying the total donor genome copies per nucleic acid volume of sample by the total volume of nucleic acid sample obtained from the nucleic acid extraction, yielding total donor genome copies obtained from the nucleic acid extraction; and (5) calculating total donor genome copies per volume of plasma from recipient by dividing total donor genome copies obtained from the nucleic acid extraction by the volume of plasma used in nucleic acid extraction.

In some embodiments, the amount of donor-derived cell-free nucleic acids is determined as a ratio of donor-derived cell-free nucleic acids to total donor-specific and recipient-specific nucleic acids based on quantitation by dPCR assay of both donor-specific nucleic acids and recipient-specific nucleic acids in the sample. In some embodiments, the number of copies of donor genomes as determined by dPCR assay (e.g., as a number of copies of donor genomes per μL of reaction) is adjusted to the number of copies of each donor-specific target in the dPCR assay (e.g., for a two-copy target corresponding to the donor, the number of copies of donor genomes as determined by dPCR assay is divided by two). The number of copies of recipient genomes as determined by dPCR assay (e.g., as a number of copies of recipient genomes per μL of reaction) may be adjusted as described above for the number of copies of donor genomes. A ratio of donor genome copies to the total combined number of copies of recipient genomes and donor genomes is then determined. In some cases, a fraction or a percent of donor genome copies to the total combined number of copies of recipient genomes and donor genomes is determined.

B. Organ, Tissue or Cell Transplant Rejection Status

Following transplantation of, e.g., an organ, tissue, or cells, the recipient may experience immune quiescence, which is a state that can be characterized by the absence or low levels of immune activity or absence of rejection-associated clinical symptoms, such as biopsy-confirmed rejection, or significant changes in organ function, e.g., as indicated by elevated serum creatinine levels, decreased estimated glomerular filtration rate, abnormal echocardiogram results or some other clinical concern that indicates a clinical need for a biopsy.

Alternatively, the recipient may experience active rejection, which may be due to T cell-mediated rejection (TCMR), antibody-mediated rejection (ABMR or AMR), or a combination of the two (i.e., a “mixed” rejection). ABMR refers to antibody-mediated rejection of an organ, tissue or cell transplant, including, but not limited, to acute active antibody-mediated rejection, chronic active antibody-mediated rejection, and chronic stable antibody-mediated rejection. TCMR refers to cellular or T-cell mediated rejection of an organ, tissue or cell transplant, including, but not limited to TCMR IA, IB, IIA, IIB, borderline TCMR and chronic TCMR.

Accordingly, in some embodiments, the status of an organ, tissue or cell transplant as determined by any of the methods provided herein may be absence of rejection, for example, characterized by immune quiescence. In other embodiments, the status of an organ, tissue or cell transplant as determined by any of the methods provided herein may be the presence of rejection, such as active rejection, T cell-mediated rejection, antibody-mediated rejection, or mixed rejection. In further embodiments, the status of an organ, tissue or cell transplant as determined by any of the methods provided herein may be a predicted risk or likelihood of rejection, such as active rejection, T cell-mediated rejection, antibody-mediated rejection, or mixed rejection.

Detecting Transplant Rejection

In some embodiments, the methods provided herein comprise detecting, predicting, diagnosing and/or monitoring transplant rejection status of an organ, tissue or cell transplant from a donor in a transplant recipient. In some embodiments, the methods comprise detecting transplant rejection if the amount of donor-derived cell-free nucleic acids in cell-free nucleic acids in a sample obtained from the transplant recipient (e.g., as determined according to the methods of the disclosure) exceeds a predetermined threshold.

A predetermined threshold generally refers to any predetermined level or range of levels that is indicative of the presence or absence of a condition, or the presence or absence of a risk or likelihood of the presence or absence of a condition, such as a rejection or non-rejection. The predetermined threshold according to the disclosure can take a variety of forms. It can be a single cut-off value, such as a median or mean. As another example, a predetermined threshold can be determined from baseline values before the presence of a condition, or risk or likelihood of the presence of a condition, or after a course of treatment. Such a baseline can be indicative of a normal or other state in the transplant recipient not correlated with the risk or condition that is being tested for. For example, the baseline value may be the level of donor-derived cell-free nucleic acids, such as donor-derived cell-free DNA, in samples from a transplant recipient prior to transplantation, which would presumably be zero or negligible, but may also indicate baseline error in the system. In some embodiments, the predetermined threshold can be a baseline value of the transplant recipient being tested. The predetermined threshold, as it pertains to demarcating significant changes in the amounts of donor-derived cell-free nucleic acids (e.g., DNA) in a transplant recipient, may vary considerably. One of skill in the art would recognize appropriate parameters and means for determining significant changes in the amounts of donor-derived cell-free nucleic acids (e.g., DNA) in a transplant recipient over time. Once appropriate analysis parameters are selected, determining changes in the amount of donor-derived cell-free nucleic acids (e.g., DNA) in the transplant recipient over a period of time can inform the status of the transplant.

In some embodiments, an increase in the amount of donor-derived cell-free nucleic acids, e.g., DNA, in the transplant recipient over time is indicative of transplant rejection or the risk or likelihood of transplant rejection, a need for administering or adjusting immunosuppressive therapy, immunosuppressive treatment nephrotoxicity, infection, and/or a need for further investigation of the transplant status. Without wishing to be bound by theory, it is believed that if the amount of donor-derived cell-free nucleic acids, e.g., DNA, is increasing in a transplant recipient over time, then the cells of the transplant are increasingly experiencing apoptosis and/or necrosis over time, which is indicative of transplant rejection or the risk or likelihood of transplant rejection.

In some embodiments, a decrease in the amount of donor-derived cell-free nucleic acids, e.g., DNA, in the transplant recipient over time is indicative of transplant tolerance, a need for adjusting immunosuppressive therapy (e.g., decreasing), and/or a need for further investigation of the transplant status. Without wishing to be bound by theory, it is believed that if the amount of donor-derived cell-free nucleic acids, e.g., DNA, is decreasing in a transplant recipient over time, then the cells of the transplant are decreasingly experiencing apoptosis and/or necrosis over time, which is indicative of transplant tolerance, overimmunosuppression, or appropriate immunosuppression.

In some embodiments, no change in the amount of donor-derived cell-free nucleic acids, e.g., DNA, in the transplant recipient over time is indicative of stable transplant rejection status and/or opportunity for adjusting immunosuppressive therapy. Without wishing to be bound by theory, it is believed that if the amount of donor-derived cell-free nucleic acids, e.g., DNA, is not changing in a transplant recipient over time, then the cells of the transplant are experiencing a steady-state level of apoptosis over time, which is indicative of a stable transplant rejection status. A stable transplant rejection status may inform the status of the transplant during the time window analyzed, but may not inform the direction, either towards rejection or tolerance, the transplant is progressing toward. For example, the transplant may be undergoing active rejection in the transplant recipient, but a stable transplant rejection status indicates that the rate of rejection is not changing during the time it was analyzed (i.e. the rate of transplant rejection is not increasing or decreasing). Similarly, the transplant may be undergoing active tolerance in the transplant recipient, but a stable transplant rejection status indicates that the rate of tolerance is not changing during the time it was analyzed (i.e. the rate of transplant tolerance is not increasing or decreasing).

C. Administering or Adjusting Immunosuppressive Therapies

In some embodiments, the methods provided herein comprise treating transplant rejection of an organ, tissue or cell transplant from a donor in a transplant recipient, and/or adjusting immunosuppressive therapy in a transplant recipient of an organ, tissue or cell transplant from a donor.

Immunosuppressive therapy generally refers to the administration of an immunosuppressant or other therapeutic agent that suppresses immune responses in a transplant recipient. The medical practice of immunosuppression in transplant recipients has evolved to include a regimen of prophylactic pharmacologic agents, typically beginning with induction therapies to deplete lymphocytes, followed by maintenance drugs intended to inhibit activation or replication of lymphocytes such as corticosteroids, calcineurin inhibitors (such as tacrolimus), and additional inhibitors of lymphocyte replication (such as mycophenolate mofetil). After transplantation, the dosage of immunosuppressant(s) may be reduced over time to reduce the incidence and severity of side effects, such as increased risk of infectious diseases, while still avoiding immune rejection of the transplant.

In some embodiments, immunosuppressive therapy may be administered, or recommended to be administered, in response to the identification of rejection of an organ, tissue or cell transplant, including T cell-mediated rejection (TCMR), antibody-mediated rejection (ABMR or AMR), or mixed rejection of the organ, tissue or cell transplant. In other embodiments, immunosuppressive therapy dosage or frequency may be increased, or recommended to be increased, in response to the identification of rejection, or risk or likelihood of rejection, of an organ, tissue or cell transplant, including T cell-mediated rejection (TCMR), antibody-mediated rejection (ABMR or AMR), or mixed rejection of the organ, tissue or cell transplant. For example, in response to the identification of TCMR, bolus steroid treatment may be initiated (or recommended to be initiated) or maintenance immunosuppressive therapy may be increased (or recommended to be increased) with respect to dosage and/or frequency. In another example, in response to the identification of ABMR, plasmapheresis or intravenous immunoglobulin (IVIg) may be initiated (or recommended to be initiated).

In other embodiments, immunosuppressive therapy dosage or frequency may be maintained, or recommended to be maintained, in response to the identification of absence of rejection of an organ, tissue or cell transplant, e.g., immune quiescence. In other embodiments, immunosuppressive therapy dosage or frequency may be reduced, or recommended to be reduced, in response to the identification of absence of rejection of an organ, tissue or cell transplant, e.g., immune quiescence. In other embodiments, immunosuppressive therapy dosage or frequency may be stopped or discontinued, or recommended to be stopped or discontinued, in response to the identification of absence of rejection of an organ, tissue or cell transplant, e.g., immune quiescence.

In other embodiments, the methods of the disclosure may comprise modifying an immunosuppressive therapy with respect to the agent(s) administered or recommended to be administered. In some embodiments, an immunosuppressive therapy may be substituted for (or be recommended to be substituted for) another immunosuppressive therapy, e.g., in response to the identification of rejection of an organ, tissue or cell transplant, including T cell-mediated rejection (TCMR), antibody-mediated rejection (ABMR or AMR), or mixed rejection. In other embodiments, an immunosuppressive therapy may administered (or be recommended to be administered) in combination with one or more additional immunosuppressive therapies, e.g., in response to the identification of rejection of an organ, tissue or cell transplant, including T cell-mediated rejection (TCMR), antibody-mediated rejection (ABMR or AMR), or mixed rejection.

An immunosuppressive therapy, and modifications to immunosuppressive therapies (e.g., substituting one therapy for another, or combining therapies), may be selected based on various factors, such as likelihood that the immunosuppressive therapy will alleviate or diminish rejection of the organ, tissue or cell transplant, alleviate or diminish symptoms of rejection of the organ, tissue or cell transplant, diminish any direct or indirect pathological consequences of the rejection of the organ, tissue or cell transplant, decrease the rate of organ, tissue or cell transplant rejection, ameliorate or palliate the organ, tissue or cell transplant rejection state, improve prognosis of the organ, tissue or cell transplant, or slow the progression of organ, tissue or cell transplant rejection.

It should be noted that the amount of donor-derived cell-free nucleic acids, e.g., DNA, in a transplant recipient may not be the only factor taken into consideration when determining a need, or lack thereof, to administer or adjust an immunosuppressive therapy. For example, for a transplant recipient exhibiting both rejection of a transplant and increasing severity of an infection, it may not be advisable to increase or even maintain the current immunosuppressive therapy. It should thus be noted that immunosuppressive therapy being administered to a transplant recipient may be increased, decreased, or maintained irrespective of the determined amount of donor-derived cell-free nucleic acids, e.g., DNA, in the transplant recipient depending on the presence or absence of other controlling or contributing clinical factors. Thus, additional factors that may be considered when selecting, administering, modifying or adjusting an immunosuppressive therapy include prevention, control, amelioration or diminishment of undesirable effects of the immunosuppressive therapy, such as adverse events, undesirable side effects, toxicities, undesirable drug-drug interactions, undesirable symptoms, and the like.

Exemplary immunosuppressive therapies that may be used according to the methods of the disclosure include, but are not limited to, calcineurin inhibitors, mTor inhibitors, ACE inhibitors, anticoagulants, antimalarials, β-blockers, corticosteroids, cardiovascular drugs, non-steroidal anti-inflammatory drugs (NSAIDs), and steroids including, for example, aspirin, azathioprine, B7RP-1-fc, brequinar sodium, campath-1H, celecoxib, chloroquine, coumadin, cyclophosphamide, cyclosporin A, DHEA, deoxyspergualin, dexamethasone, diclofenac, dolobid, etodolac, everolimus, FK778, feldene, fenoprofen, flurbiprofen, heparin, hydralazine, hydroxychloroquine, CTLA-4 or LFA3 immunoglobulin, ibuprofen, indomethacin, ISAtx-247, ketoprofen, ketorolac, leflunomide, meclophenamate, mefenamic acid, mepacrine, 6-mercaptopurine, meloxicam, methotrexate, mizoribine, mycophenolic acid derivatives, naproxen, oxaprozin, Plaquenil, NOX-100, prednisone, methylprednisolone, rapamycin (sirolimus), sulindac, tacrolimus (FK506), thymoglobulin, tolmetin, tresperimus, U0126, as well as antibodies including, for example, alpha lymphocyte antibodies, adalimumab, anti-CD3, anti-CD25, anti-CD52 anti-IL2R, anti-TAC antibodies, basiliximab, daclizumab, etanercept, hu5C8, infliximab, OKT4, and natalizumab, or any combination thereof.

Administering or Adjusting Transplant-Related Therapies

The methods of the present disclosure may also be used to inform the need to administer or adjust other transplant-related therapies. In general, an amount of donor-derived cell-free nucleic acids, e.g., DNA, beyond a predetermined threshold in the transplant recipient may be informative with regard to determining a need to administer or adjust other transplant-related therapies.

Other transplant-related therapies include treatments or therapies besides transplantation or immunosuppressive therapy that are administered to a recipient of a transplant to promote survival of the transplant or to treat transplant-related symptoms (e.g., cytokine release syndrome, neurotoxicity). Examples of other transplant-related therapies include, but are not limited to, administration of antibodies, antigen-targeting ligands, non-immunosuppressive drugs, and other agents that stabilize or destabilize components of transplants that are critical to transplant activity or that directly activate or inhibit one or more transplant activity. These activities may include the ability to induce, modify or attenuate an immune response, recognize particular antigens, replicate, and/or induce repair of damaged tissues.

Adjusting the Monitoring of a Transplant Recipient

The methods of the present disclosure may also be used to inform the need to adjust monitoring of the recipient of the transplant. In general, an amount of donor-derived cell-free nucleic acids, e.g., DNA, beyond a predetermined threshold in the transplant recipient may be informative with regard to determining a need to adjust monitoring of a recipient of a transplant. In some embodiments, determining the status of the transplant, as described above, is informative with regard to determining a need to adjust monitoring of a recipient of a transplant.

Depending on the status of the transplant, monitoring of the recipient may be adjusted accordingly. For example, monitoring may be adjusted by increasing or decreasing the frequency of monitoring, as appropriate. Monitoring may be adjusted by altering the means of monitoring, for example, by altering the metric or assay that is used to monitor the recipient.

D. Reporting

In some embodiments, the methods described herein can include generating and/or providing a report.

In some embodiments, the report comprises an assessment or diagnosis of organ, tissue or cell transplant rejection status in a transplant recipient, e.g., determined according to the methods described herein. In some embodiments, the report indicates the presence of rejection of an organ, tissue or cell transplant, including T cell-mediated rejection (TCMR), antibody-mediated rejection (ABMR or AMR), or mixed rejection of the organ, tissue or cell transplant. In some embodiments, the report indicates the risk or likelihood of rejection of an organ, tissue or cell transplant, including T cell-mediated rejection (TCMR), antibody-mediated rejection (ABMR or AMR), or mixed rejection of the organ, tissue or cell transplant. In some embodiments, the report indicates the absence of rejection of an organ, tissue or cell transplant, e.g., immune quiescence. In some embodiments, the report comprises a recommendation to administer an immunosuppressive therapy to the transplant recipient, for example, based, at least in part, on detection of rejection of an organ, tissue or cell transplant according to the methods provided herein, including T cell-mediated rejection (TCMR), antibody-mediated rejection (ABMR or AMR), or mixed rejection of the organ, tissue or cell transplant, e.g., as described in greater detail above. In some embodiments, the report comprises a recommendation to adjust or modify an immunosuppressive therapy being administered to the transplant recipient, for example, based, at least in part, on the detection of the presence or absence of rejection of an organ, tissue or cell transplant according to the methods provided herein, e.g., a recommendation to increase, decrease, or maintain a dose or frequency, or discontinue or modify the immunosuppressive therapy, as described in greater detail above.

In some embodiments, the report indicates the presence of rejection of an organ, tissue or cell transplant in the recipient (e.g., including T cell-mediated rejection (TCMR), antibody-mediated rejection (ABMR or AMR), or mixed rejection of the organ, tissue or cell transplant), and comprises a recommendation to administer an immunosuppressive therapy to the transplant recipient, e.g., as described in greater detail above. In some embodiments, the report indicates the presence of rejection of an organ, tissue or cell transplant (e.g., including T cell-mediated rejection (TCMR), antibody-mediated rejection (ABMR or AMR), or mixed rejection of the organ, tissue or cell transplant), and comprises a recommendation to adjust an immunosuppressive therapy being administered to the transplant recipient, e.g., to increase a dose or frequency, or modify the immunosuppressive therapy, as described in greater detail above. In some embodiments, the report indicates the risk or likelihood of rejection of an organ, tissue or cell transplant in the recipient (e.g., including T cell-mediated rejection (TCMR), antibody-mediated rejection (ABMR or AMR), or mixed rejection of the organ, tissue or cell transplant), and comprises a recommendation to administer an immunosuppressive therapy to the transplant recipient, e.g., as described in greater detail above. In some embodiments, the report indicates the risk or likelihood of rejection of an organ, tissue or cell transplant (e.g., including T cell-mediated rejection (TCMR), antibody-mediated rejection (ABMR or AMR), or mixed rejection of the organ, tissue or cell transplant), and comprises a recommendation to adjust an immunosuppressive therapy being administered to the transplant recipient, e.g., to increase a dose or frequency, or modify the immunosuppressive therapy, as described in greater detail above. In some embodiments, the report indicates the absence of rejection of an organ, tissue or cell transplant, and comprises a recommendation to adjust or modify an immunosuppressive therapy being administered to the transplant recipient, e.g., to maintain or decrease a dose or frequency, or discontinue or modify the immunosuppressive therapy, as described in greater detail above.

A report according to the present disclosure may be in any suitable form, such as in digital, electronic, web-based, or paper form. In some embodiments, the report may be provided to one or more parties, such as the transplant recipient, a caregiver, a physician, a hospital, a clinic, a third-party payor, an insurance company, a government office, and any combination thereof.

In some embodiments, the report may include information on prognosis, or potential or suggested therapeutic options. The report can include information on the likely effectiveness of a therapeutic option, the acceptability of a therapeutic option, or the advisability of applying the therapeutic option to the transplant recipient. For example, the report can include information, or a recommendation on, the administration of a drug, such as an immunosuppressive therapy, as well as recommended dosages or treatment regimens, and/or in combination with other drugs.

In some embodiments, the report may be generated and/or provided to a party within less than one day, or within any of about 1 day, about 2 days, about 3 days, about 4 days, about 5 days, about 6 days, about 7 days, about one week, about two weeks, about three weeks, about four weeks, or about one month, from obtaining or receiving the sample from the transplant recipient, or from determining the status of the organ, cell or tissue transplant (e.g., the presence or absence of rejection of the organ, cell or tissue transplant).

### II. Cell-Free Nucleic Acids

The methods provided herein involve the analysis of cell-free nucleic acids (e.g., cell-free DNA, RNA, mRNA, miRNA, double-stranded DNA, single-stranded DNA, single-stranded DNA hairpins, DNA/RNA hybrids, RNA hairpins, and combinations thereof) from a transplant recipient to detect and monitor transplant rejection status of an organ, tissue or cell transplant in the transplant recipient.

Cell-free nucleic acids generally refer to nucleic acids that are present or circulating outside of a cell, such as, for example, nucleic acids that are present in a bodily fluid (e.g., blood, plasma, serum, urine, etc.) of a transplant recipient. Cell-free nucleic acids may have originated from various locations within a cell. For example, cell-free nucleic acids, such as cell-free DNA, may have originated from, e.g., nuclear DNA and/or mitochondrial DNA. Without wishing to be bound by theory, it is believed that cell-free nucleic acids are released from cells via apoptosis or necrosis of the cells (i.e., cell death). Accordingly, and without wishing to be bound by theory, it is believed that during transplant rejection, apoptosis or necrosis of cells from the organ, tissue or cell transplant will result in donor-derived cell-free nucleic acids being released into the bodily fluids of a transplant recipient. Transplant recipients undergoing transplant rejection may then have a cell-free nucleic acid population in their bodily fluids which includes both their own endogenous cell-free nucleic acids (recipient-derived cell-free nucleic acids) as well as cell-free nucleic acids that originated from the donor (donor-derived cell-free nucleic acids). Thus, as disclosed herein, assessing the levels of donor-derived cell-free nucleic acids in a transplant recipient according to the methods of the present disclosure may be used to detect, predict, diagnose and/or and monitor the status of an organ, tissue or cell transplant.

In some embodiments, the methods of the disclosure comprise determining an amount of donor-derived cell-free nucleic acids in a sample comprising cell-free nucleic acids from a transplant recipient, e.g., according to any of the detection/quantification methods provided herein. As described in greater detail below, the sample from the transplant recipient may be, or be derived from, any bodily fluid, including whole blood, plasma, serum, lymph, urine, buccal swabs, bone marrow, saliva, sweat, lung lavage, tears, ear flow fluid, sputum, fluid bone marrow suspension, semen, vaginal flow, cerebrospinal fluid, brain fluid, ascites, milk, secretions of the respiratory tract, intestinal tract fluids, or genitourinary tract fluids. In some embodiments, the sample is, or is derived from, urine. In some embodiments, the sample is, or is derived from, whole blood or a fraction thereof (e.g., serum or plasma). In some embodiments, the sample is, or is derived from, plasma. In some embodiments, cell-free nucleic acids present in the sample may be entirely recipient-derived, or may include a mixture of recipient-derived and donor-derived nucleic acids.

In some embodiments, the donor-derived cell-free nucleic acids are donor-derived cell-free DNA, and/or the recipient-derived cell-free nucleic acids are recipient-derived cell-free DNA. In some embodiments, the donor-derived cell-free nucleic acids are donor-derived cell-free RNA, and/or the recipient-derived cell-free nucleic acids are recipient-derived cell-free RNA. In some embodiments, cell-free RNA comprises tissue-specific RNA transcripts. In some embodiments, cell-free RNA from a transplant recipient may be analyzed for gene expression levels to detect, predict, diagnose and/or monitor the status of an organ, tissue or cell transplant.

A. Extraction

Cell-free nucleic acids, such as cell-free DNA or cell-free RNA, from a sample from a transplant recipient may be extracted prior to analysis according to the methods of the disclosure. Methods for extraction of cell-free nucleic acids, such as cell-free DNA or cell-free RNA, are known in the art, see e.g. Current Protocols in Molecular Biology, latest edition. Exemplary, non-limiting methods that may be used include the Triton-Heat-Phenol (THP) method (Xue et al., (2009) Clin. Chim. Acta 404, 100-104), the phenol-chloroform isoamyl alcohol isolation (PCI) protocol (Yuan et al., (2012) Yonsei Med. J. 53:132; Schmid et al., (2005) Clin. Chem. 51, 1560-1561; and Hufnagl et al., (2013) J. Nucleic Acids Investig. 4, 1-3), and the salting-out method (Miller et al., (1988) Nucleic Acids Res. 16:1215; Jorgez et al., (2006) Genet. Med. 8, 615-619). Other exemplary, non-limiting methods of extracting cell-free nucleic acids are known in the art, see, e.g., Cell-Free Plasma DNA as a Predictor of Outcome in Severe Sepsis and Septic Shock. Clin. Chem. 2008, v. 54, p. 1000-1007; Prediction of MYCN Amplification in Neuroblastoma Using Serum DNA and Real-Time Quantitative Polymerase Chain Reaction. JCO 2005, v. 23, p. 5205-5210; Circulating Nucleic Acids in Blood of Healthy Male and Female Donors. Clin. Chem. 2005, v. 51, p. 1317-1319; Use of Magnetic Beads for Plasma Cell-free DNA Extraction: Toward Automation of Plasma DNA Analysis for Molecular Diagnostics. Clin. Chem. 2003, v. 49, p. 1953-1955; Chiu R W K, Poon L L M, Lau T K, Leung T N, Wong E M C, Lo Y M D. Effects of blood-processing protocols on fetal and total DNA quantification in maternal plasma. Clin Chem 2001; 47: 1607-1613; and Swinkels et al. Effects of Blood-Processing Protocols on Cell-free DNA Quantification in Plasma. Clinical Chemistry, 2003, vol. 49, no. 3, 525-526. Any methods known in the art for extracting cell-free nucleic acids may be used in the methods of the present disclosure. In some embodiments, extraction of cell-free nucleic acids, such as cell-free DNA or cell-free RNA, from a sample may be performed using commercially-available kits, such as the Qiagen QIAamp Circulating Nucleic Acid Kit, BioChain cfPure Cell-Free DNA Extraction Kit, ThermoFisher Scientific MagMax Cell-Free DNA Isolation Kit, Roche MagNa Pure 24 system, Zymo Quick-cfDNA Serum & Plasma Kit, or Macherey-Nagel NucleoSnap cfDNA kit, NucleoSpin Gel and PCR Clean-Up kit or NucleoSpin Plasma XS kit. In some embodiments, extraction of cell-free nucleic acids, such as cell-free DNA or cell-free RNA, from a sample is performed using the Qiagen QIAamp Circulating Nucleic Acid Kit.

In some embodiments, cell-free nucleic acids, such as cell-free DNA or cell-free RNA, may be further assessed for quality, quantity (e.g., concentration or absolute amounts), and/or fragment size, for example, prior to analysis according to the methods of the disclosure. Quantification, quality analysis and/or fragment size analysis of cell-free nucleic acids may be performed using any method known in the art, such as fluorometric dsDNA assays (e.g., using a Quant-iT PicoGreen dsDNA Assay Kit from ThermoFisher), using a fluorometer such as using a Qubit assay, real-time PCR, quantitative PCR, or digital droplet PCR (see, e.g., Rago et al. (2007) Cancer Res. 67, 9364-9370; Takai et al. (2015) Sci. Rep. 5:18425; Rostami et al., (2020) Cell Rep. 31:107830; Heet al. (2019) Sci. Rep. 9: 5599), using a bioanalyzer (e.g., using a High Sensitivity DNA microchip kit (Agilent Technologies) and an Agilent 2100 Bioanalyzer). In some embodiments, cell-free nucleic acids may also be assessed for the presence or absence of modifications such as methylation. Any suitable method for analyzing modifications of nucleic acids known in the art may be used, such as bisulfite-based methylation analysis, for example, using kits such as the EpiTect Plus DNA Bisulfite Kit or PyroMark PCR Kit from Qiagen.

In some embodiments, at least about any of 10 ng, 20 ng, 30 ng, 40 ng, 50 ng, 100 ng, 200 ng, 300 ng, 400 ng, 500 ng, 600 ng, 700 ng, 800 ng, 900 ng, 1000 ng, 2000 ng, 3000 ng, 4000 ng, 5000 ng, or more, of cell-free nucleic acids (e.g., cell-free DNA or RNA) may be obtained from a sample from a transplant recipient, e.g., after extraction. In some embodiments, between about 50 ng and about 5000 ng, between about 100 ng and about 4000 ng, or between about 200 ng and about 4000 ng (including any value within each of the recited ranges) of cell-free nucleic acids (e.g., cell-free DNA or RNA) may be obtained from a sample from a transplant recipient, e.g., after extraction. In some embodiments, cell-free nucleic acids (e.g., cell-free DNA or RNA) for use in the methods of the disclosure may have a concentration of at least about any of 0.01 ng/μL, 0.1 ng/μL, 0.5 ng/μL, 0.75 ng/μL, 1 ng/μL, 5 ng/μL, 10 ng/μL, 15 ng/μL, 20 ng/μL, 25 ng/μL, 30 ng/μL, 35 ng/μL, 40 ng/μL, 45 ng/μL, 50 ng/μL, 55 ng/μL, 60 ng/μL, 65 ng/μL, 70 ng/μL, or more, e.g., after extraction. In some embodiments, cell-free nucleic acids (e.g., cell-free DNA or RNA) for use in the methods of the disclosure may have a concentration of between about 1 ng/μL and about 100 ng/μL, between about 1 ng/μL and about 70 ng/μL, or between about 4 ng/μL and about 70 ng/μL, including any value within each of the recited ranges, e.g., after extraction.

B. Amplification

Cell-free nucleic acids, such as cell-free DNA or cell-free RNA, isolated from samples obtained from a transplant recipient may be amplified for downstream techniques and analysis, e.g., according to the methods of the disclosure.

Methods of amplifying nucleic acids are well-known in the art. Amplification generally refers to any device, method or technique that can generate copies of a nucleic acid. Amplification of cell-free nucleic acids (e.g., cell-free DNA or RNA) may involve, for example, isothermal amplification techniques such as Loop-mediated isothermal amplification (LAMP), and polymerase chain reaction (PCR) techniques such as linear amplification (cf. U.S. Pat. No. 6,132,997), rolling circle amplification, and the like. Cell-free nucleic acids (e.g., cell-free DNA or RNA) may be amplified for use in downstream analysis by, for example, qPCR, dPCR or sequencing. In instances where the cell-free nucleic acids are cell-free RNA, the methods may comprise a step of generating DNA from the cell-free RNA, e.g., using a reverse transcriptase. The Fluidigm Access Array™ System, the RainDance Technologies RainDrop system, or other technologies for multiplex amplification may be used for multiplex or highly parallel simplex nucleic acid amplification. Amplification may involve the use of high-fidelity polymerases such as, for example, FastStart High Fidelity (Roche), Expand High Fidelity (Roche), Phusion Flash II (ThermoFisher Scientific), Phusion Hot Start II (ThermoFisher Scientific), KAPA HiFi (Kapa BioSystems), or KAPA2G (Kapa Biosystems).

Amplification may include an initial PCR cycle that adds a unique sequence to each individual molecule, called molecular indexing. Amplified nucleic acids may also be subjected to additional processes, such as indexing (also referred to as barcoding or tagging). Methods of indexing nucleic acids are well-known in the art and are described herein. Indexing allows for the use of multiplex-sequencing platforms, which are compatible with a variety of sequencing systems, such as Illumina HiSeq, MiSeq, and ThermoFisher Scientific Ion PGM and Ion Proton. Multiplex sequencing permits the sequencing of nucleic acids from multiple samples in a single reaction through the use of indexing to specifically identify the sample source of the sequenced nucleic acids.

### III. Samples

In some embodiments, the methods provided herein comprise determining an amount of donor-derived cell-free nucleic acids in one or more samples, e.g., one or more biological samples, from a transplant recipient, such as one or more samples comprising cell-free nucleic acids.

In general, a sample according to the methods provided herein can be (or can be derived from) any body fluid, including samples of whole blood, plasma, serum, lymph, peripheral blood mononuclear cells, urine, buccal swabs, bone marrow, saliva, sweat, lung lavage, tears, ear flow fluid, sputum, fluid from bone marrow suspension, semen, vaginal flow, cerebrospinal fluid, brain fluid, ascites, milk, secretions of the respiratory tract, intestinal tract fluids, or genitourinary tract fluids. In some embodiments, the sample is, or is derived from, urine. In some embodiments, the sample is, or is derived from, whole blood or a fraction thereof (e.g., serum or plasma). In some embodiments, the sample is, or is derived from, plasma. In some embodiments, the sample is, or is derived from, serum. In some embodiments, where the sample is (or is derived from) blood, serum or plasma, the blood, serum or plasma is derived from the venous or arterial blood of the transplant recipient. In some embodiments, the blood, serum or plasma is derived from the venous blood of the transplant recipient.

In some embodiments, a sample from a transplant recipient according to the present disclosure comprises cell-free nucleic acids. In some embodiments, cell-free nucleic acids present in the sample may be entirely recipient-derived cell-free nucleic acids, or cell-free nucleic acids present in the sample may include a mixture of recipient-derived cell-free nucleic acids and donor-derived cell-free nucleic acids.

In some embodiments, the methods provided herein comprise providing the sample from the recipient. In some embodiments, the methods provided herein further comprise obtaining the sample from the recipient, e.g., in ways and into specially prepared containers that prevent degradation and/or contamination of the sample and/or analytes. Once a sample is obtained, it may be used directly, frozen, or otherwise stored in a condition that maintains the integrity of the sample (e.g., of nucleic acids in the sample, such as cell-free nucleic acids in the sample) and prevents degradation and/or contamination of the sample. The amount of a sample that is taken at a particular time may vary, and may depend on additional factors, such as any need to repeat analysis of the sample. In some embodiments, up to 50, 40, 30, 20, 10, 9, 8, 7, 6, 5, 4, 3, 2, 1, 0.5, 0.1 mL of a sample is obtained. In some embodiments, 0.1-1, 1-50, 2-40, 3-30, or 4-20 mL of a sample is obtained. In some embodiments, more than 1, 5, 10, 15, 20, 25, 30, 35, 40, 45, 50, 55, 60, 65, 70, 75, 80, 85, 90, 95, or 100 mL of a sample may be obtained.

Samples may be obtained from a recipient of a transplant once, or more than once. Where multiple samples are obtained from a recipient of a transplant, the frequency of sampling may vary. For example, samples may be obtained about once every day, about once every other day, about once every three days, about once every week, about once every two weeks, about once every three weeks, about once every month, about once every two months, about once every three months, about once every four months, about once every five months, about once every six months, about once every year, or about once every two years, or more, after the initial sampling event. In some embodiments, any of one, two, three, four, five, ten, twenty, thirty, forty, fifty, sixty, seventy, eighty, ninety, one hundred, two hundred, three hundred, four hundred, or five hundred, or more samples may be obtained from the recipient. In some embodiments, any of between one and five, between five and ten, between ten and twenty, between twenty and thirty, between thirty and forty, between forty and fifty, between fifty and sixty, between sixty and seventy, between seventy and eighty, between eighty and ninety, between ninety and one hundred, between one hundred and two hundred, between two hundred and three hundred, between three hundred and four hundred, or between four hundred and five hundred, or more, samples may be obtained from the recipient.

In some embodiments, one or more samples may be obtained from a recipient of a transplant over a time interval for use in determining, i.e. detecting, predicting, diagnosing and/or monitoring, the status of an organ, cell or tissue transplant in the recipient according to the methods of the present disclosure. The time interval during which samples are taken from the recipient of a transplant following the transplantation event may vary. Exemplary intervals for sampling are described, for example, in U.S. application Ser. No. 14/658,061, which is hereby incorporated by reference in its entirety. For example, samples may be taken from a transplant recipient at various times and over various periods of time for use in determining, i.e. detecting, predicting, diagnosing and/or monitoring, the status of an organ, cell or tissue transplant in the recipient according to the methods of the present disclosure. In some embodiments, one or more samples may be taken from the transplant recipient prior to the recipient receiving the organ, cell or tissue transplant. In some embodiments, one or more samples may be taken from the transplant recipient any of, or any combination of any of, about 1 day, about 2 days, about 3 days, about 4 days, about 5 days, about 6 days, about 7 days, about 1 week, about 2 weeks, about 3 weeks, about 4 weeks, about 1 month, about 2 months, about 3 months, about 4 months, about 5 months, about 6 mounts, about 7 months, about 8 months, about 9 months, about 10 months, about 11 months, about 12 months, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, or more, after the transplant event or after an initial sample has been taken from the recipient (i.e., after an initial sampling event). In some embodiments, samples may be taken from the transplant recipient within about three months after, about six months after, about nine months after, or less than one year after the transplant event. In some embodiments, samples may be taken from the transplant recipient at various times before the one-year anniversary of the transplant event, at the one-year anniversary of the transplant event, or at various times after the one year anniversary of the transplant event. For example, at the one-year anniversary after a transplant, samples may be taken from the transplant recipient starting at month 12 (i.e., the one-year anniversary of the transplant event) and continuously for periods of time after this. In some embodiments, the time period for obtaining samples from a transplant recipient is within the first few days after the transplant from the donor to the recipient occurred. This may be done to monitor induction therapy. In some embodiments, the time period for obtaining samples from a transplant recipient includes during tapering of an immunosuppressive regimen being administered to the recipient, a period that typically occurs during the first 12 months after the transplant from the donor to the recipient has occurred. In some embodiments, the time period for obtaining samples from a transplant recipient includes during an initial long term immunosuppressive maintenance phase, which may begin about 12-14 months, or earlier or later, after the transplant from the donor to the recipient has occurred. In some embodiments, the time period for obtaining samples from a transplant recipient includes during the entire long-term maintenance of an immunosuppressive regimen, which may be any time beyond 12 months, or earlier or later, after the transplant from the donor to the recipient has occurred.

In some embodiments, one or more samples are obtained from a recipient of a transplant twice per week in the first three weeks after transplantation. In some embodiments, one or more samples are obtained daily for the first 1 or 2 weeks following transplantation. In some embodiments, one or more samples are obtained once per week for the first three months after transplantation. In some embodiments, one or more samples are obtained once per month for the first year after transplantation. In some embodiments, one or more samples are obtained four times per year after the first year after transplantation.

In some embodiments, samples are obtained from a recipient of a transplant for one to three consecutive months, starting at the one-year anniversary of the transplantation event (i.e., 12 months after the transplantation event), providing a total of four to six samples for analysis taken over a three month time interval, with samples being collected about every two weeks. In some embodiments, a recipient of a transplant has samples taken once per week for one to three consecutive months, starting at the one-year anniversary of the transplantation event (i.e., 12 months after the transplantation event), providing a total of twelve samples for analysis taken over a three month time interval. The total duration of obtaining samples from a recipient of a transplant, as well as the frequency of obtaining such samples, may vary and will depend on a variety of factors, such as clinical progress. For example, a recipient of a transplant may have samples obtained for the duration of their lifetime. Appropriate timing and frequency of sampling will be able to be determined by one of skill in the art for a given recipient of a transplant.

In some embodiments, the methods provided herein comprise providing one or more samples from the recipient, e.g., for use according to the methods of the disclosure.

### IV. Donors and Recipients

The methods of the disclosure comprise detecting, predicting, diagnosing and/or monitoring transplant rejection status of an organ, tissue or cell transplant from a donor in a transplant recipient; treating transplant rejection of an organ, tissue or cell transplant from a donor in a transplant recipient; and/or adjusting immunosuppressive therapy in a transplant recipient of an organ, tissue or cell transplant from a donor.

In some embodiments, an organ, tissue or cell transplant according to the present disclosure is a cross-species transplant (i.e., a xenogeneic or heterologous transplant), wherein the organ, tissue or cell transplant donor is of a different species from the transplant recipient. Accordingly, in some embodiments, the methods of the disclosure comprise detecting, predicting, diagnosing and/or monitoring the status of a cross-species, xenogeneic or heterologous organ, tissue or cell transplant, treating transplant rejection of a cross-species, xenogeneic or heterologous organ, tissue or cell transplant, and/or adjusting immunosuppressive therapy in a transplant recipient of a cross-species, xenogeneic or heterologous organ, tissue or cell transplant, wherein the organ, tissue or cell transplant donor is of a different species from the transplant recipient.

In some embodiments, the transplant recipient and the transplant donor are animals. In some embodiments, the transplant recipient and/or the transplant donor are vertebrates. In some embodiments, the transplant recipient and/or the transplant donor are mammals. In some embodiments, the transplant recipient and/or the transplant donor are non-human mammals. In some embodiments, the transplant recipient is a human and the transplant donor is a non-human animal, such as a non-human vertebrate or a non-human mammal. In some embodiments, the transplant recipient is a non-human animal, such as a non-human vertebrate or a non-human mammal, and the transplant donor is a humanized non-human organ. In some embodiments, the non-human animal is a non-human primate, a cow, a horse, a pig, a sheep, a goat, a dog, a cat, a rodent, a bird, a reptile, or other non-human animal. In some embodiments, the non-human mammal is a non-human primate, a cow, a horse, a pig, a sheep, a goat, a dog, a cat, a rodent, or other non-human mammal. In some embodiments, the rodent is a mouse, a rat, a squirrel, a prairie dog, a porcupine, a beaver, a guinea pig, a hamster, or another rodent. In some embodiments, the non-human primate is a monkey or an ape. In some embodiments, the non-human primate is a chimpanzee, a bonobo, a gorilla, an orangutan, a rhesus monkey, a cynomolgus monkey, a pig-tailed monkey, an African green monkey, a marmoset, a capuchin monkey, a spider monkey, a vervet monkey, a baboon, a squirrel monkey, an owl monkey, or another non-human primate. In some embodiments, the transplant recipient is a human, and the transplant donor is a pig. In some embodiments, the pig is a Sus scrofa, Sus scrofa domesticus, Phacochoerus aethiopicus, Potamochoerus porcus porcus, Potamochoerus porcus, Babirousa babyrussa species. In some embodiments, the pig is a Hanford pig, a mini- or micro-pig, Yucatan pig, Yucatan micro pig, Sinclair pig, Gottingen pig, Duroc pig, Yorkshire pig, Landrace pig, or a combination, hybrid or cross-species thereof.

In some embodiments, the donor and/or the recipient are genetically modified, or comprise one or more cells, organs or tissues that are genetically modified. In some embodiments, the organ, tissue or cell transplant is genetically modified. Such genetic modifications may include, for example, gene inactivations or complete gene knock-outs; and/or genetic integrations or expression of recipient or donor, or non-recipient or non-donor, genes that the original genome of the transplant-donor did not possess.

In some embodiments, the transplant is derived from an adult (e.g., an adult human or non-human animal). In some embodiments, the transplant is derived from a fetus, an embryo, embryonic stem cells, induced pluripotent stem cells, a child, or a teenager, juvenile or adolescent. In some embodiments, the transplant is derived from a male or a female.

### V. Organ, Cell and Tissue Transplants

The methods of the disclosure comprise detecting, predicting, diagnosing and/or monitoring transplant rejection status of an organ, tissue or cell transplant from a donor in a transplant recipient.

In some embodiments, a transplant according to the present disclosure is a transplant of one or more organs. In some embodiments, the organ is a solid organ or a hollow organ. In some embodiments, the transplant is a whole organ, or portions of organ(s). In some embodiments, the organ may be any of a kidney, a pancreas, a liver, a heart, a lung, an intestine, a bladder, an adrenal gland, an appendix, a brain, an ear, an esophagus, an eye, a gall bladder, a urinary bladder, a small or large intestine, a mouth, a muscle, a nose, a parathyroid gland, a pineal gland, a pituitary gland, a spleen, a stomach, a thymus, a thyroid gland, a trachea, a uterus, or a vermiform appendix. In some cases, the organ is a gland organ. For example, the organ may be an organ of the digestive or endocrine system. In some cases, the organ can be both an endocrine gland and a digestive organ. In some cases, the organ may be derived from endoderm, ectoderm, primitive endoderm, or mesoderm. In some embodiments, the transplant is a vascularized composite graft. In some embodiments, the organ transplant is an intact organ, a fragment of an intact organ, a disrupted organ, or a cell from an organ.

In some embodiments, the transplant is a kidney transplant. A kidney transplant may also be referred to as a renal transplant. In some embodiments, the kidney transplant is derived from a deceased donor or a living donor. In some embodiments, a kidney is transplanted together with a pancreas. In some embodiments, the kidney transplant is derived from a pig donor.

In some embodiments, the transplant is a heart transplant. A heart transplant may also be referred to as a cardiac transplant. In some embodiments, the heart transplant is derived from a pig donor.

In some embodiments, a transplant according to the present disclosure is a transplant of one or more cells. In some embodiments, the one or more cells are taken directly from a donor for administration into a recipient; the one or more cells are taken from a donor and genetically engineered before administration into a recipient; the one or more cells are taken from a donor and cultured before administration into a recipient; the one or more cells are taken from a donor and subjected to a manufacturing process before administration into a recipient; or any combination thereof. The one or more cells may also be stored before administration into a recipient (e.g., “off-the-shelf” cells). In some embodiments, the one or more cells are blood cells, stem cells, cardiomyocytes, neurons, lymphocytes, natural killer (NK) cells, NK T cells, T regulatory (T-reg) cells, neurons, macrophages, dendritic cells, pancreatic islet cells, or any combination thereof. In some embodiments, the blood cells may include hematopoietic stem cells (i.e., HSCs), T cells, B cells, chimeric antigen receptor (CAR) T cells, NK cells, NK T cells, tumor-infiltrating lymphocytes (TILs), and any combination thereof. In some embodiments, the one or more cells are CAR T cells, universal CAR T cells (i.e., wherein the CAR binds to an antibody that binds a specific antigen), split CAR T cells (i.e., wherein a dimerizing agent activates CAR T cell function), activatable CAR T cells, repressible CAR T cells, multiphasic CAR T cells (i.e., wherein the CAR must bind multiple specific antigens and/or agents to induce T cell activation), tumor infiltrating lymphocytes, regulatory T cells, genetically modified T cells, T cells with genetically modified or synthesized T cell receptors (TCRs), virus-specific T cells (e.g., EBV, HPV, BKV, CMV, etc.), antigen-specific T cells, neoantigen-specific T cells, or any cell isolated from a donor. In some embodiments, the one or more cells are administered as bone marrow, cord blood, or purified cells. In some embodiments, the one or more cells are bone marrow cells. In some embodiments, the one or more cells are cord blood cells. In some embodiments, the transplant comprises stem cells. In some embodiments, the stem cells are administered as bone marrow, cord blood, or purified stem cells. In some embodiments, the stem cells are derived from a donor. In some embodiments, the stem cells are administered as a hematopoietic cell transplantation. In some embodiments, the blood cells comprise one or more of white blood cells (e.g., monocytes, lymphocytes, neutrophils, eosinophils, basophils, macrophages, and the like), red blood cells (erythrocytes), and platelets. In some embodiments, the blood cells are administered as a blood transfusion (e.g., whole blood transfusion), a transfusion of individual components of blood, or a transfusion of purified cells such as white blood cells (e.g., monocytes, lymphocytes, neutrophils, eosinophils, basophils, or macrophages), red blood cells (erythrocytes), or platelets. In some embodiments, the one or more cells are derived from an organ (e.g., an organ described herein or any other organ known in the art). In some embodiments, the one or more cells are pancreatic cells, hepatic cells, cardiac cells, renal cells, nervous system cells, and the like.

In some embodiments of any of the methods of the present disclosure, the transplant comprises stem cells. In some embodiments, the stem cells are embryonic, tissue-specific, induced pluripotent, hematopoietic, mesenchymal, skeletal, myogenic, cardiac, neural, epidermal, or intestinal stem cells. In some embodiments, the stem cells are hematopoietic stem cells, embryonic stem cells, adult stem cells, multipotent stem cells, pluripotent stem cells, neuronal stem cells, heart stem cells, cells derived from cord blood, or induced pluripotent stem cells.

In some embodiments, the one or more cells are cholecystocytes, cardiomyocytes, glomerulus cells (e.g., parietal, podocyte), kidney proximal tubule brush border cells, Loop of Henle thin segment cells, thick ascending limb cells, kidney distal tubule cells, kidney collecting ductal cells, interstitial kidney cells, enterocytes, goblet cells, enterocytes, caveolated tuft cells, enteroendocrine cells, ganglion neurons, parenchymal cells, non-parenchymal cells, hepatocytes, sinusoidal endothelial cells, Kupffer cells, hepatic stellate cells, myocytes, pancreatic beta cells, endothelial cells, or exocrine cells, or any combination thereof.

In some embodiments, the one or more cells are genetically engineered, and/or subjected to a manufacturing process, and/or cultured before administration into the recipient. In some embodiments, a transplant of one or more cells comprises multiple types of cells. In some embodiments, a transplant comprises one or more cells that are genetically distinguishable from each other. In some embodiments, a transplant of one or more cells comprises two or more independent administrations. For example, one cell type may be administered in a first administration, and a second cell type may be administered in second administration.

In some embodiments, a transplant according to the present disclosure is a transplant of a tissue. Exemplary tissues include, but are not limited to, connective tissue, epithelial tissue, muscular tissue, nervous tissue, lymphatic tissues, blood or blood components, fat tissue, cartilage, dense fibrous tissue, skeletal muscle, cardiac muscle, or smooth muscle. The muscle tissue may comprise muscle fibers or myocytes. In some embodiments, the tissue is a cornea, a tendon, a heart valve, a vein or artery, skin, a bone, birth tissue, or portions, fragments and/or combinations thereof. In some embodiments, the birth tissue is placental tissue, amniotic membrane tissue, chorionic membrane tissue, amniotic fluid tissue, umbilical cord tissue, umbilical veins, or Wharton's jelly. In some cases, the tissue is a bone or tendon (both referred to as musculoskeletal grafts).

### VI. Additional Analyses

The methods of the present disclosure may be performed in addition to or in conjunction with other analyses of samples from a transplant recipient and/or a transplant donor.

In some embodiments, the presence or levels of an infectious agent in the transplant recipient are tested. Infectious agents which may be tested for include, for example, viruses; bacteria such as Pseudomonas aeruginosa, Enterobacteriaceae, Nocardia, Streptococcus pneumonia, Staphyloccous aureus, and Legionella; fungi such as Candida, Aspergillus, Cryptococcus, Pneumocystis carinii; or parasites such as Toxoplasma gondii. In some embodiments, the presence or levels of viral infectious agents in the transplant recipient are tested. Viral biomarkers may be analyzed in nucleic acid obtained from a sample from the transplant recipient to determine the presence or levels of viruses in the transplant recipient. Viruses which may be tested for include, for example, Cytomegalovirus, Epstein-Barr virus, Anelloviridae, and BK virus. The results of the tests for presence or levels of viruses may be used to classify the immune status of the transplant recipient and/or to determine the status of infection in the transplant recipient. In some embodiments, immunosuppressive therapies may be decreased, or at least not increased, in transplant recipients that are classified as having a high risk of clinically significant infection. In some embodiments, immunosuppressive therapies may be increased, or at least not decreased, in transplant recipients that are classified as having a low risk of clinically significant infection. It should be noted that, as other clinical factors may inform decisions to adjust immunosuppressive therapies, a transplant recipient may have immunosuppressive therapy currently being administered to them increased, decreased, or maintained irrespective of the results of tests for presence or levels of viruses and/or classification for risk of clinically significant infection.

In some embodiments, the methods of the present disclosure comprise performing gene expression analysis in a sample from the transplant recipient. In some embodiments, the gene expression analysis is performed on peripheral blood mononuclear cells (PBMCs) from the recipient. In some embodiments, the gene expression analysis is performed on a whole blood sample, or parts thereof, from the recipient. In some embodiments, the gene expression analysis is performed on selected genes that provide information relating to the status of a cell, organ or tissue transplant in a transplant recipient. In some embodiments, the gene expression analysis is performed using any suitable method known in the art, such as RNA-sequencing, microarray methods, Nanostring analysis systems, and quantitative real-time PCR. In some embodiments, the gene expression analysis is performed using an AlloMap test. AlloMap tests involve performing quantitative real-time polymerase chain reaction (qRT-PCR) assays using RNA that has been isolated from PBMCs. The expression of a select number of genes is analyzed and this gene expression data is used to provide information relating to the status of a cell, organ or tissue transplant in a transplant recipient. The AlloMap test is known in the art. Results of the gene expression analysis (e.g., obtained using an AlloMap test or any other suitable method) may be used in conjunction with the methods of the present disclosure, with or without a method to define a single score from the combined tests, to determine the status of a cell, organ, or tissue transplant in a transplant recipient and/or inform the need to administer immunosuppressive therapy or adjust immunosuppressive therapy being administered to the transplant recipient.

In some embodiments, the methods of the present disclosure involve determining a single score that may be used to convey the status of a cell, organ, or tissue transplant in a transplant recipient.

In some embodiments, the methods of the present disclosure involve determining a combination score that may be used to convey the status of a cell, organ, or tissue transplant in a transplant recipient. Combination scores are generally calculated based on the results of multiple (e.g., two or more) assays used to probe the status of the cell, organ, or tissue transplant in the transplant recipient. For example, combination scores may be calculated based on the determined levels of donor-derived cell-free nucleic acids, e.g., DNA, in the transplant recipient and results of a gene expression profiling assay, e.g., as described above (such as, an AlloMap test). Combination scores may be calculated based on a single sample taken from a transplant recipient, or they may be based on samples taken from a transplant recipient over a time interval. Combination scores may be used to determine the status of a cell, organ, or tissue transplant in a transplant recipient and/or inform the need to administer immunosuppressive therapy or adjust immunosuppressive therapy being administered to the transplant recipient.

Additional biomarker analyses, gene expression assays, and other assays for determining, i.e., detecting, predicting, diagnosing and/or monitoring, the status of a cell, organ, or tissue transplant in a transplant recipient and/or determining the need to administer or adjust immunosuppressive therapy may also be used in addition to or in conjunction with the methods of the present disclosure, with or without a method to define a single score from the combined tests, which will be readily apparent to one of skill in the art.

Additional analyses may be performed to identify markers of new, metastatic, or recurrent cancers in transplant recipients. Primers may be designed to amplify regions where genetic mutations are known to occur to provide an early detection of cancer by identification of known tumor-associated mutations. This may be advantageous, at least in part, because transplant recipients may be at heightened risk of developing certain malignancies due to immunosuppression.

### VII. Kits of the Disclosure

Also provided herein are kits for use in any one of the methods described herein.

In one aspect, provided herein is a kit for detecting, predicting, diagnosing and/or monitoring transplant rejection status of an organ, tissue or cell transplant from a donor in a transplant recipient, wherein the donor and the recipient belong to different species. In some embodiments, the kit comprises reagents for use in accordance with a method of the present disclosure. For example, in some embodiments, the kit includes reagents for analyzing cell-free nucleic acids isolated from a transplant recipient, as described herein. In some embodiments, the kit may comprise reagents for performing qPCR, dPCR, or a sequencing method (such as NGS or HTS), e.g., as described above. Such reagents may include primers (e.g., for use in a qPCR or dPCR method), probes or dyes (e.g., for use in a qPCR or dPCR method), reagents for sequencing library preparation, reagents for amplifying nucleic acids (e.g., polymerases, buffers, etc.), reagents for performing nucleic acid extraction from samples, if extraction is necessary, and the like. In some embodiments, the kit comprises one or more PCR reaction oligonucleotide primers and probe sets that hybridize to donor-specific or recipient-specific target sequences, e.g., in cell-free nucleic acids from a transplant recipient. In some embodiments, the one or more PCR reaction oligonucleotide primers and probe sets of the kit are for use in qPCR or digital PCR quantitation of donor-derived cell-free nucleic acids as absolute copies of donor-derived cell-free nucleic acids in sample or in reference to total cell-free nucleic acids analyzed. In some embodiments, the one or more PCR reaction oligonucleotide primers and probe sets of the kit are for use in qPCR or digital PCR quantitation of donor-derived cell-free nucleic acids as absolute copies of donor-derived cell-free nucleic acids in the sample, or as a ratio of donor-derived cell-free nucleic acids to total donor-derived and recipient-derived cell-free nucleic acids in the sample. In some embodiments, the one or more PCR reaction oligonucleotide primers and probe sets of the kit are for use in digital PCR quantitation of donor-derived cell-free nucleic acids in reference to the amount of sample analyzed or in reference to total cell-free nucleic acids analyzed. In some embodiments, the kit may comprise reagents sufficient to analyze a single sample. In some embodiments, the kit may comprise reagents sufficient to analyze several samples.

In some embodiments, the kit may include instructions to specify target values and control materials that may be used in conjunction with the reagents and instructions provided in the kit. In some embodiments, the kit further includes controls for use in accordance with a method of the present disclosure. For example, in some embodiments the kit further comprises control samples comprising known amounts of nucleic acids (e.g., cell-free nucleic acids).

In some embodiments, the kit further includes instructions for use in accordance with a method of the present disclosure. Instructions for performing any one of the methods described herein may be included. For example, in some embodiments, the kit comprises instructions for use of the kit and for data analysis to determine an amount of donor-derived cell-free nucleic acids.

In some embodiments, the kit further includes instructions and specifications for input material quality or input preparation methods.

In some embodiments, the kit comprises software instructions for analysis of sequence data (e.g., sequence reads) or PCR data (e.g., data from qPCR or dPCR), e.g., to determine an amount of donor-derived cell-free nucleic acids in the cell-free nucleic acids obtained from a sample from a transplant recipient. In some embodiments, the kit comprises instructions for accessing software that may be used to perform statistical analysis of donor-derived cell-free nucleic acids in the cell-free nucleic acids obtained from a sample from a transplant recipient, for example, instructions for downloading and/or installing the software.

### VIII. Software, Systems and Devices

Certain aspects of the methods described herein for detecting, predicting, diagnosing and/or monitoring transplant rejection status of an organ, tissue or cell transplant from a donor in a transplant recipient, wherein the donor and the recipient belong to different species, may be carried out by a computer system or device. For example, in some embodiments, steps of analyzing sequence data (e.g., sequence reads) or PCR data (e.g., data from qPCR or dPCR) to determine an amount of donor-derived cell-free nucleic acids in cell-free nucleic acids obtained from samples from a transplant recipient may be performed using a computer system or device. Also provided herein is software for carrying out the methods described herein.

The above-described embodiments of the present disclosure may be implemented in a variety of ways. For example, some aspects of the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general-purpose hardware (e.g., one or more processors) that is programmed using microcode or software to perform the functions recited above.

In this respect, it should be noted that implementation of various features of the present disclosure may use at least one non-transitory computer-readable storage medium (e.g., a computer memory, a floppy disk, a compact disk, a tape, etc.) encoded with a computer program (i.e., a plurality of instructions), which, when executed on a processor, performs the above discussed functions and methods. The computer-readable storage medium can be transportable such that the program stored thereon can be loaded onto any computer resource to implement certain aspects of the present disclosure discussed herein. In addition, it should be noted that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement certain aspects of the present disclosure.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Methods for Non-Invasively Monitoring Organ Transplant Health in Cross-Species Transplantation

This Example describes digital polymerase chain reaction (dPCR)- and sequencing-based methods for non-invasive monitoring of cross-species organ transplants using cell-free DNA (cfDNA) from the transplant recipient.

In the methods described below for cross-species transplantation monitoring, a genetically modified pig served as an exemplary organ donor and a human as an exemplary organ recipient. The pig had been genetically modified (Revivicor, a United Therapeutics subsidiary) to not express four porcine genes and to additionally express six human genes. These modifications were considered and found to represent negligible alterations with respect to donor genome size. Therefore, all calculations herein were based on the pig genome, as publicly available for Sus scrofa.

In general, modifications of the genome of a transplant-donating animal may include gene inactivations or complete gene knock-outs, and/or genetic integrations and expression of additional human or non-human genes that the original genome of the transplant-donating animal did not possess. Most if not all genetic modifications serve the purpose to mitigate a potentially aggressive immune response triggered by the transplant recipient's immune system.

dPCR-Based Methods for Non-Invasive Monitoring of Cross-Species Organ Transplants

### General Approach

Single-copy target detection and multi-copy target detection assays were designed for use in both singleplex and multiplex dPCR assays, as outlined in Table 1. Accuracy was calculated as percentage of measured copies over expected copies, based on input DNA, as measured by Qubit.

TABLE 1
Target Detection Design Overview For
Singleplex and Multiplex dPCR Assays.

| dPCR | Recipient Target Type, | Donor Target Type, | Method |
|---|---|---|---|
| Type | number (n) | numbe r(n) | # |
| Single- | Single copy, n = 1 | Single copy, n = 1 | 1A |
| plex | Single copy, n = 1 | Multi-copy, n = 1 | 1B |
| | Multi-copy, n = 1 | Single copy, n = 1 | |
| | Multi-copy, n = 1 | Multi-copy, n = 1 | |
| Multi- | Single copy, n = 1 OR n > 1 | Single copy, n = 1 OR n > 1 | 2A |
| plex | Single copy, n = 1 OR n > 1 | Multi-copy, n = 1 OR n > 1 | 2B |
| | Multi-copy, n = 1 OR n > 1 | Single copy, n = 1 OR n > 1 | |
| | Multi-copy, n = 1 OR n > 1 | Multi-copy, n = 1 OR n > 1 | |

Primer and Probe Design

PCR primers and probes were designed to quantitatively detect human-specific genomic DNA sequences and pig-specific genomic DNA sequences. Exemplary PCR primers and fluorophore-labelled probes are shown below in Table 2. Genomic DNA sequences in gene intronic regions were favorably selected as at least part of targeted regions for PCR primer design because they are more likely to show diversity between the pig and human genomes. Primer and probe designs were chosen using, for example, the Primer3 program (see, primer3.org), with targeted assay parameters of: amplicon size of 60-110 base pairs, primer melting temperature (Tₘ) of 60° C., and probe Tₘ of 65-70° C.

TABLE 2
Exemplary PCR primers and fluorophore-labelled
probes to detect and quantitate human-specific
genomic DNA sequences and pig-specific genomic
DNA sequences. In one embodiment of the invention,
the two-copy pig target assay, as shown herein,
detected two copies of the PCR targets per haploid
genome. In another embodiment of the invention,
the 8-copy pig target assay, as shown herein,
detected 8 copies of the same PCR target per
haploid genome.

| Assay | Forward primer | Reverse primer | Probes |
|---|---|---|---|
| Human | AACTACATGGTGA | CTGCCCATCAGCCAG | FAM- |
| assay A | GTGCTACATG | GTG (SEQ ID NO: | TGAGCCCCAAAGCTGGTGTG |
| (1-copy | (SEQ ID NO: 1) | 2) | G-BHQ (underlined |
| target) | | | sequence corresponds |
| | | | to SEQ ID NO: 5) |
| Pig assay | ACTGTTGGAAGTGC | TTCTGAGAGACCTCT | FAM- |
| A (2-copy | ATGTGC (SEQ ID | GCTTGG (SEQ ID | CTGCTGCTCCTTGCTCTCAC |
| target) | NO: 3) | NO: 4) | TGCAGG-BHQ |
| | | | (underlined sequence |
| | | | corresponds to SEQ |
| | | | ID NO: 6) |
| Human | CCCCACCGGAGGAG | GCGAAAGCCGAAGTC | 56-FAM- |
| assay B | TTTTG (SEQ ID | TAGGG (SEQ ID | CGGTCTGTA/ZEN/GAGAGC |
| (1 copy) | NO: 7) | NO: 8) | TATGCAGGGTGAGGGC- |
| | | | 3IABKFQ (underlined |
| | | | sequence corresponds |
| | | | to SEQ ID NO: 9) |
| Pig | CCAGCTTCATTTTG | CCCCTTTTCAGGGAA | 5Cy5- |
| assay B | CCCTCG (SEQ ID | TGCTC (SEQ ID | CCAGCACCC/TAO/CGATGC |
| (1 copy) | NO: 10) | NO: 11) | GGACGAGTCG-3IAbRQSp |
| | | | (underlined sequence |
| | | | corresponds to SEQ |
| | | | ID NO: 12) |
| Pig | TAGGTCATTGCTTT | TTCACAGGGACGGAA | 5Cy5- |
| assay C | CCGGCT (SEQ ID | AACCG (SEQ ID | CTGGCTGGC/TAO/GCGCGG |
| (1 copy) | NO: 13) | NO: 14) | CGAGAACTT-3IAbRQSp |
| | | | (underlined sequence |
| | | | corresponds to SEQ |
| | | | ID NO: 15) |
| Human | GGAGAAAAGCCTAC | TGACGTCACCCGTTC | 5SUN- |
| assay C | CGCACA (SEQ ID | TGTTG (SEQ ID | ACCGGCAGG/ZEN/TGGCCC |
| (1 copy) | NO: 16) | NO: 17) | CACCCTGCAT-3IABKFQ |
| | | | (underlined sequence |
| | | | corresponds to SEQ |
| | | | ID NO: 18) |
| Pig | TGTTGCGGCAAGAT | TTCATTCCCACTGAC | 56-FAM- |
| assay D | GCGT (SEQ ID | CAGATG (SEQ ID | TCCCAGAGCTTGCCCAGAG |
| (8-copy | NO: 19) | NO: 20) | C-3IABkFQ |
| target) | | | (underlined sequence |
| | | | corresponds to SEQ |
| | | | ID NO: 21) |

Reporter dyes: FAM, Cy5, SUN, FAMN.
Quenchers: ZEN, TAO, IABkFQ, 3IAbRQSp, BHQ

To ensure the absence of cross-species reactions, PCR primers were checked, using an in silico PCR program, against one genome, e.g., pig, for a unique amplicon product, and against the other genome, e.g., human, for the absence of amplicon products. The target genome, e.g., pig genome, was computationally divided into base pair (bp) segments, such as segments of, e.g., 80 bp, and the segments were mapped back to the target genome to identify multiply-mapping segments. The multiply-mapping segments were aligned to the non-target genome, and any alignments were discarded. The remaining multiply-mapping segments were in some cases filtered to remove fragments, e.g., fragments with high or low GC content or fragments that aligned to repeat-masked regions, or to remove, for example, undesirable regions such as non-exonic regions. “LOC” genes (i.e., genes of uncertain function, with no official symbol and/or no ortholog) were discarded and overlapping segments were merged. The remaining segments were submitted to Primer3 design software and the 0th primer set output for each segment was validated by in silico PCR to confirm multiple hits in the target genome and absence of hits in the non-target genome. Among designs produced by Primer3, only those with a minimum separation of 1 kilobase between segments were chosen for experimental evaluation.

Probes were labeled with either the same fluorescent dye for singleplex dPCR assays, or different fluorescent dyes for multiplex dPCR assays (as described below).

For multi-copy target detection assays, pig-specific multigene families were identified, and PCR primers/probes were designed to detect multiple copies of the target sequences in the pig genome, but to not detect anything in the human genome. For pig-specific PCR assay primers and probe designs, genomic sequences of multigene families (e.g., rRNA, Hox, Histone, and tubulin gene families) were searched for regions with high diversity between the pig and human genomes. In comparison to single-copy target detection assays, multi-copy target detection assays have an increased number of pig genome positive partitions which is important when pig cfDNA is present at very low copies in the sample. For example, a pig two-copy target detection assay detects two pig target copies per haploid genome and, consequently, generates twice as many pig-specific partition counts compared to a single-copy target detection assay.

Method 1A: Singleplex dPCR Assays Detecting a Single-Copy Human-Specific Target and a Single-Copy Pig-Specific Target.

Various amounts, e.g., up to 120 ng, of cfDNA input per sample are assayed separately with a human-specific dPCR assay detecting a single-copy human-specific target, and a pig-specific dPCR assay detecting a single-copy pig-specific target. The assays are performed on a Stilla Naica system using 1× multiplex PCR MasterMix, 0.5 μM forward- and reverse-primers, and 0.3 μM FAM-labeled probes in a 25-μl reaction on Crystal Droplet dPCR Sapphire Chips according to the manufacturer's recommended standard protocol. dPCR results are acquired with a Crystal Droplet dPCR Reader, and are analyzed with the Crystal Droplet dPCR Miner software. Pig and human genome copy concentrations are normalized to input cfDNA amount (ng), and percent pig genome copies are calculated relative to total copies of pig and human genomes in the sample.

Method 1B: Singleplex dPCR Assays Detecting a Single-Copy Human-Specific Target and a Multi-Copy Pig-Specific Target.

Separate singleplex dPCR assays detecting a single-copy human-specific target, and a multi-copy pig-specific target were performed, as described above for Method 1A. To determine the pig genome copy concentration, the measured pig copy number was divided by the number of copies of the pig-specific target per haploid genome (e.g., the measured pig copy number was divided by 2 if a two-copy target was used in the pig assay). Pig and human genome copies per μl were added together, and percent pig genome copies were calculated relative to total copies of pig and human genomes in the sample. Multi-copy pig-specific target detection assays improve quantitation precision and sensitivity.

Method 2A: A Multiplex dPCR Assay Detecting 1 or More Single-Copy Human-Specific Targets and 1 or More Single- and/or Multi-Copy Pig-Specific Targets Using Unique Fluorescent Dyes to Distinguish all Different Targets or to Differentiate the Two Species.

Individual human target-specific and pig target-specific assays (e.g., as described above for Methods 1A and 1B) were selectively combined into a single multiplex assay with different fluorescent dye labels (e.g., FAM, HEX, ATTO 550, ROX, Cy5, Cy5.5, SUN) for the different component assay probes. The genome copy concentrations from each component were corrected for genome copy concentration for multi-copy target assays (if any), averaged for human and for pig genome copy concentrations, and percent pig genome copies were calculated relative to total copies of pig and human genomes in the sample. Thus, in Method 2A, more targets were used to increase the number of positive partitions for the donor and improve assay precision and sensitivity.

Method 2B: A Multiplex dPCR Assay Detecting 1 or More Single-Copy Human-Specific Targets and at Least 1 Multi-Copy Pig-Specific Target Using Non-Unique Fluorescent Dyes for Targets within the Same Species.

Multiplex dPCR assays were designed as described above for Method 2A, except that at least 1 multi-copy target assay was used in a single dPCR assay reaction, either labeled with different fluorescence dyes or labeled with the same fluorescent dye. Thus, in Method 2B, either multiple fluorescence channels or a single fluorescence channel were used to detect the multiple pig-specific target PCR assays to increase the number of positive partitions for the determination of pig donor-derived cfDNA, and to improve assay precision and sensitivity, assuming different genome targets are apart from each other and can be amplified independently in different droplets in the dPCR assay.

Data Analysis

For Methods 1A-1B and 2A-2B, as described above, the outputs from the dPCR reactions (i.e., the pig-specific target sequence copies per μL and the human-specific target sequence copies per μL of reaction) were calculated for genome copies per μL concentrations after dividing each of them with the number of target sequence copies per haploid genome per assay. For relative concentrations, the fraction or percent of pig genome copies was calculated relative to the total genomes, e.g., the total number of human and pig genome copies.

The number of pig genomes can also be determined and reported as an absolute value per unit sample input, e.g., as the number of pig genome copies per ng of cfDNA or per milliliter (mL) of plasma.

For absolute concentrations, the pig target sequence copies/μl from the dPCR instrument were first adjusted (divided) by the number of target sequence copies per target in the genome. Then, using the volume that was used in the dPCR reaction, in addition to the volume of eluted DNA from extraction and volume of plasma used in DNA extraction, pig genome equivalent cfDNA copies per ml plasma were calculated. Alternative methods to determine absolute concentrations include, but are not limited to, obtaining pig (target) copies per μl (cp/μl) from the dPCR instrument, calculating pig genome cp/μl by dividing the pig cp/μl with the number of target copy per haploid genome and number of assays (if more than 1 assay are combined in a fluorescent channel), determining pig genome copies into the dPCR reaction, determining pig genome cp/ml plasma. Specific steps for one method of determining the absolute number of pig-derived sequences were as follows:
1. Take pig target copies/μl from dPCR instrument and divide by the number of pig target copies per haploid genome (if more than 1 pig target assay is combined in a fluorescent channel, also divide by the number of pig target assays in the fluorescent channel), yielding pig target copies/μl in the reaction;
2. Calculate total pig genome copies in the reaction by multiplying pig target copies/μl in the reaction by volume of reaction;
3. Divide the total pig genome copies in the reaction by the volume of DNA sample used in the reaction, yielding total pig genome copies per volume of DNA sample;
4. Multiply the total pig genome copies per DNA volume of sample by the total volume of DNA sample obtained from the DNA extraction, yielding total pig genome copies obtained from the DNA extraction; and
5. Calculate total pig genome copies per volume of plasma from recipient by dividing total pig genome copies obtained from the DNA extraction by the volume of plasma used in DNA extraction.

### Sequencing-Based Methods for Non-Invasive Monitoring of Cross-Species Organ Transplants

Library Preparation and Sequencing

Between 25-100 ng cfDNA were used to prepare shotgun libraries using Illumina's DNA Prep Kit® according to the manufacturer's instructions.

Average library fragment size was determined by Tapestation® cfDNA kit, and the library was quantified by Qubit. Libraries were sequenced on MiSeq (2×65 or 2×55 cycles, 12 pM) or on NextSeq (2×65 or 2×35 cycles, 1.3-1.8 pM) with paired end reads. Pure human genomic DNA and pure pig genomic DNA were also sequenced along with contrived samples at between 0.2% and 0.4% of pig genome equivalents (GE).

Bioinformatics

A next-generation sequencing (NGS) shotgun pipeline was used to analyze sequencing data and to quantify the amount of pig-donor-derived cell-free DNA in samples obtained from the human recipient of a heart transplant donated by an engineered pig and from human deceased model recipients of a pig heart transplant.

Differences in genome sizes between the human genome and the genome of the transplant-donating animal, e.g. the genome of the genetically modified pig, were accounted for by either genome size-based normalizations for total reads, or utilizing average coverage across defined bin sizes of the genomes, as described in greater detail below.

A combined reference FASTA file was created consisting of the 24 chromosomes of the human genome (UCSC hg19; genome.ucsc.edu/), and the 20 chromosomes of the pig genome (build Sus scrofa 11.1). The raw sequence reads from the FASTQ files were aligned to the combined reference using a BWA aligner. Read alignment was done with a minimum alignment score cutoff of 90%, allowed percent gaps set to 5%, and with no split alignment.

Various mapping filter models were applied to filter aligned reads to determine estimation biases and stringency (see, Table 3). For each model listed in Table 3, average coverage across 1 megabase (Mb) regions was calculated for both human and pig genomes. Percent pig donor-derived cfDNA was computed as: mₚ/(mₚ+mₕ)*100, wherein mₚ is the median of average coverage across 1 Mb regions on the pig genome and mₕ is the median of average coverage across 1 Mb regions on the human genome. This methodology accounts for the fact that the pig genome is smaller than the human genome.

Alternatively, percent pig donor-derived cfDNA was computed by aligning the sequence reads to a combined reference that encompasses porcine genome as well as human genome as described above, and determining the percent of the sequence reads that align to the porcine genome as a fraction of the combined reference. This methodology does not account for the difference in size between the human and pig genomes.

In silico samples were prepared at varying fractions of human and pig genomes, with 25 million total sequencing reads to determine the accuracy of the models in Table 3.

TABLE 3
Models for filtering aligned reads.

| Model | Read filter |
|---|---|
| M1 | All aligned reads. |
| M2 | M1 after excluding reads with multiple matches. |
| M3 | M2 after excluding mate missing, translocated reads, |
| | and reads with improper orientation. |
| M4 | M2 after excluding reads with alignment score < 95%. |
| M5 | M2 after excluding reads with alignment score < 98%. |
| M6 | M2 after excluding partially aligned reads (i.e., soft clipped reads). |
| M7 | M2 after excluding partially aligned reads (i.e., hard and soft |
| | clipped reads, alignments with MAPQ smaller than 10). |

Differences were observed in the mean fragment size of pig-derived versus human-derived cfDNA. For example, as shown in Table 4, analysis of two samples containing pig-derived and human-derived cfDNA fragments showed that human-derived cfDNA fragments have longer mean fragment sizes. The mean cfDNA fragment size difference between pig and human (e.g., as shown in Table 4) could result in over-estimation of the pig-donor-derived cfDNA fraction, for example, if smaller cfDNA fragments lead to greater coverage. Accordingly, a correction factor may be applied to account for differences in the mean fragment size of pig versus human cfDNA to improve accuracy.

TABLE 4
Pig and human cfDNA fragment size analysis.

| | Human cfDNA mean | Pig cfDNA mean |
|---|---|---|
| Sample | fragment size (bp) | fragment size (bp) |
| A | 106.3 | 93.7 |
| B | 103.1 | 98.1 |

### Example 2: Non-Invasive Monitoring of Organ Transplant Health in a Human Recipient of a Pig Heart Transplant

This Example describes non-invasive monitoring of organ transplant health in a human recipient of a pig heart transplant using the dPCR-based and sequencing-based methods described in Example 1 herein.

### Samples

Blood from the human recipient was collected in two 10 mL Streck tubes at days 6, 13, 19, 25, 33, 46, 49, 55, and 60 after the human recipient had received an investigational heart (UHeart) transplant from a pig that was genetically modified by Revivicor, a United Therapeutics subsidiary. Cell-free DNA (cfDNA) was extracted using a Qiagen QIAamp Circulating Nucleic Acid Kit and quantified using Qubit.

As shown in Table 5, cfDNA yields varied over time after the organ transplantation, from 223 ng on day 25 after the transplantation to 3360 ng on day 46 after the transplantation.

TABLE 5
Patient cfDNA yields.

| | Qubit | |
|---|---|---|
| Days After | Concentration | |
| Transplantation | (ng/uL) | Total Yield (ng) |
| 6 | 17.3 | 865 |
| 13 | 8.23 | 412 |
| 19 | 5.1 | 255 |
| 25 | 4.46 | 223 |
| 33 | 20 | 1000 |
| 46 | 67.2 | 3360 |
| 49 | 31.5 | 1575 |
| 55 | 7.73 | 386.5 |
| 60 | 19.95 | 997.5 |

dPCR-Based Analyses

Control samples containing known percentages of control pig and human genomic DNA were generated to compare the performance of dPCR assays using a single-copy pig-specific target, or a two-copy pig-specific target. As shown in Table 6, dPCR assays using a two-copy pig-specific target resulted in twice as many positive dPCR partitions as compared to dPCR assays using a single-copy pig-specific target.

TABLE 6
Results of dPCR assays using a two-copy pig-specific
target compared to a single-copy pig-specific target
(using control samples with human and pig DNA).

| | Number of positive dPCR partitions for pig target | | |
|---|---|---|---|
| Sample (known | 1-copy pig | 2-copy pig | Weighted |
| percent pig DNA) | target assay | target assay | average |
| A (1% pig) | 8.67 | 15.8 | 8.3 |
| B (0.2% pig) | 1.42 | 3.65 | 1.6 |
| C (0.2% pig) | 2.45 | 4.01 | 2.2 |
| D (0.1% pig) | 0.62 | 1.71 | 0.7 |
| E (0% pig) | 0 | 0 | 0.0 |

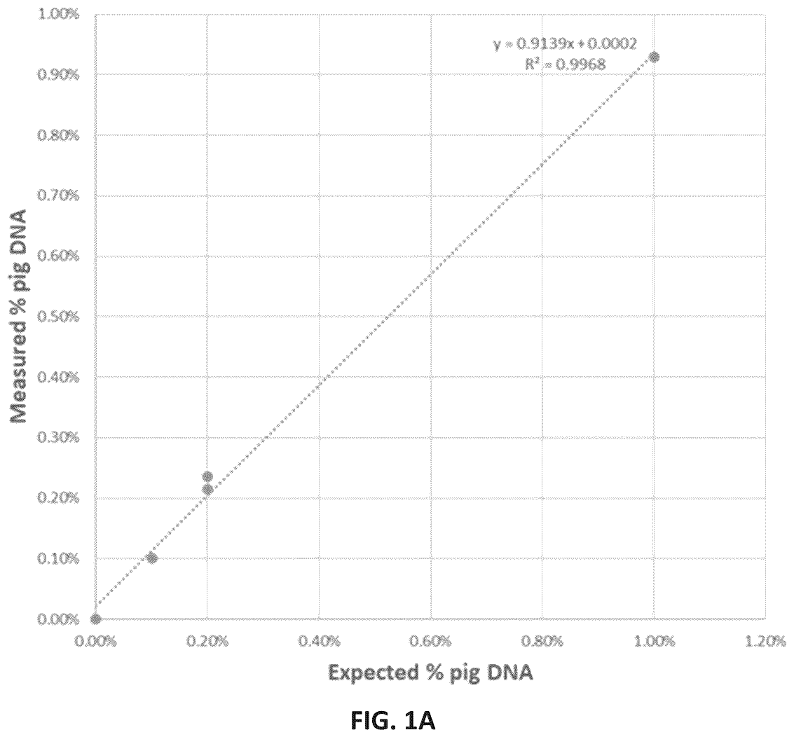

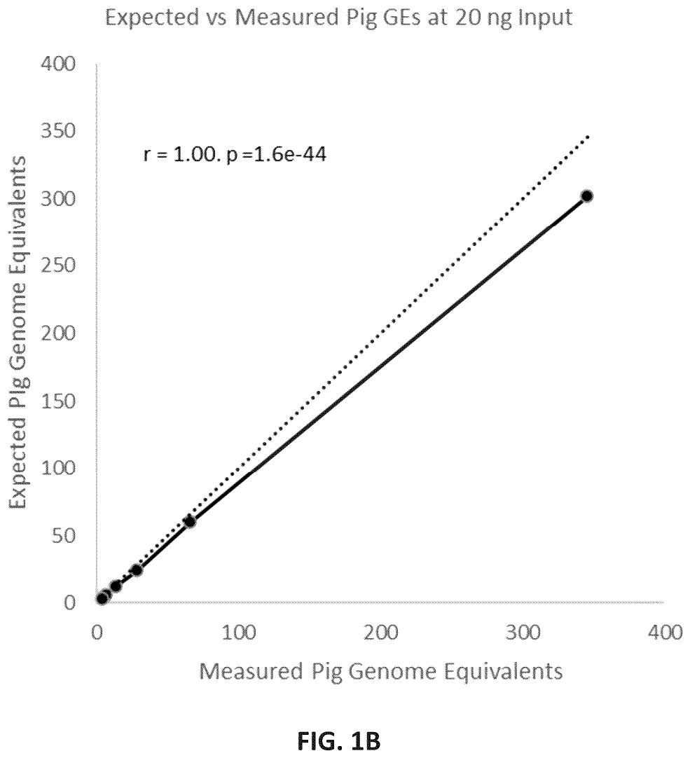

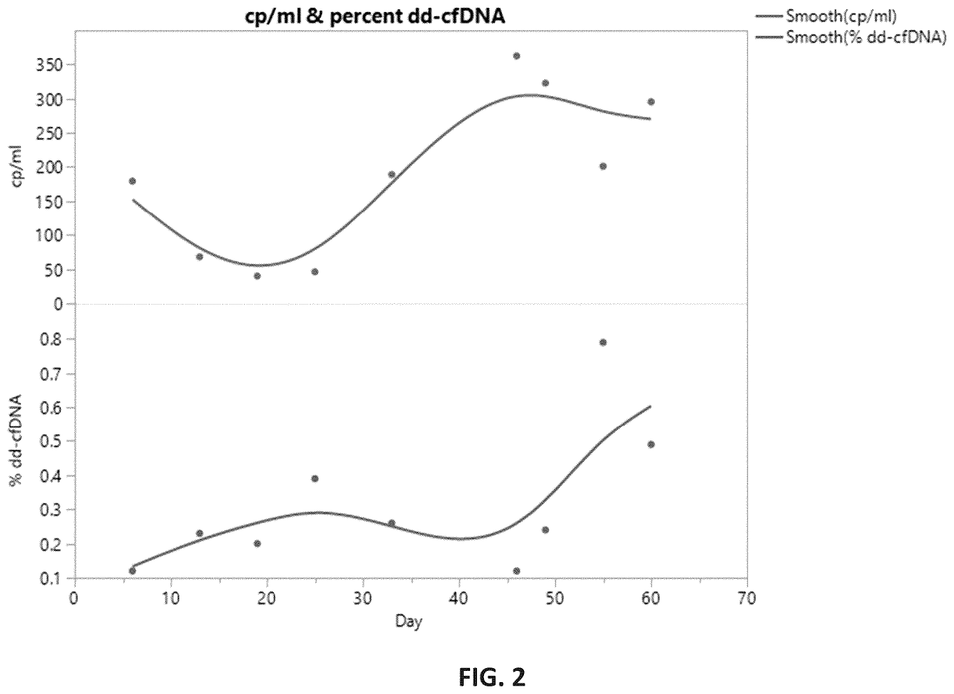

As shown in FIGS. 1A and 1B, singleplex dPCR assays using a two-copy pig-specific target (see FIG. 1A) as well as the multiplex dPCR assays using two one-copy pig-specific targets (see FIG. 1B) resulted in linear relationships between the known or expected percentage of control pig DNA and the calculated or measured percentage of pig DNA. Cell-free DNA (cfDNA) samples from the human recipient of the pig heart transplant were analyzed using dPCR at five timepoints post-transplantation (i.e., at days 6, 13, 19, 25, 33, 46, 49, 55, and 60 post-transplantation). As shown in FIG. 2 for genomic copies/mL plasma sample and percentage of pig donor-derived cell-free DNA, the percentage of pig donor-derived cell-free DNA was lowest at day 6 after the organ transplantation, and increased over time until reaching a peak at day 55 after the organ transplantation, which was followed by a slight decrease at day 60 after the organ transplantation.

### Sequencing-Based Analyses

cfDNA samples from the human recipient of the pig heart transplant were also analyzed with shotgun next-generation sequencing (NGS) using filtering models M1-M7 as described in Example 1 herein (see, Table 3). Control samples containing pure human and pig genomic DNA were also analyzed using the same methods.

As shown in Table 7, with filtering model M7, the pure human DNA control samples had a calculated percent pig DNA of 0%, suggesting that the Limit of Blank (LOB) was very close to zero.

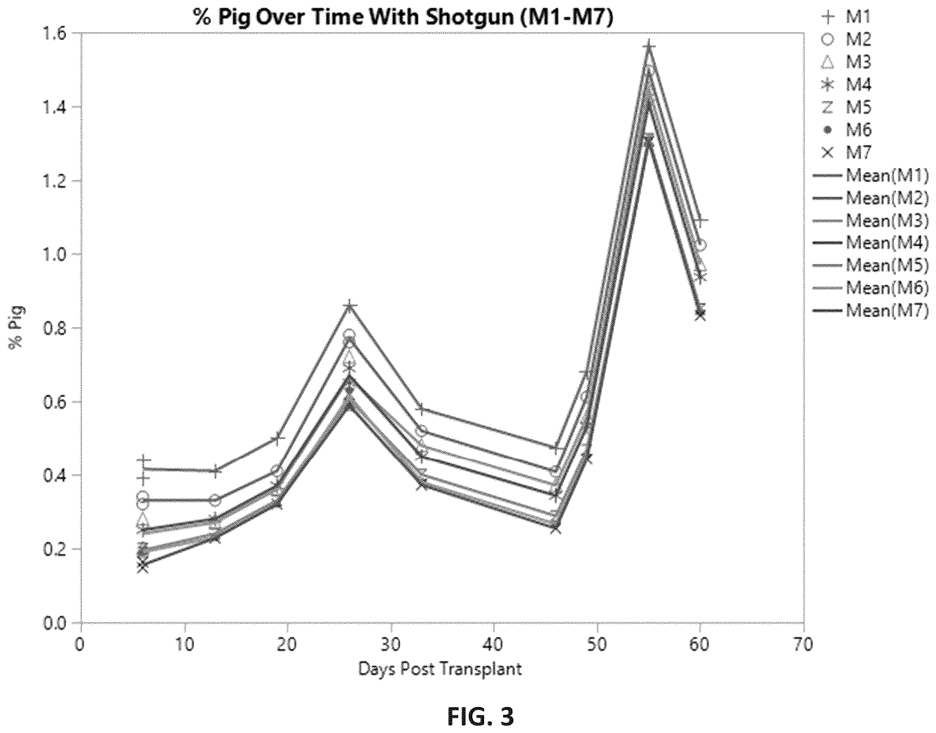

Filtering models M1-M7, as described in Table 3 of Example 1 herein, resulted in different percent pig-donor-derived cfDNA estimates for samples obtained from the human recipient of the pig heart transplant (see, Table 7 and FIG. 3). However, all of the filtering models showed a consistent trend over time, with the percent of pig donor-derived cfDNA being lowest at day 6 after the organ transplantation, and increasing over time until reaching a peak at day 55 after the organ transplantation, which was followed by a slight decrease at day 60 after the organ transplantation (see, Table 7 and FIG. 3).

TABLE 7
Shotgun NGS results from control samples (pure human or pig genomic
DNA samples), and patient cfDNA samples from a human recipient of
a pig heart transplant on the indicated days after the transplantation.

| | | Total | | | | | | | |
|---|---|---|---|---|---|---|---|---|---|
| | | Number | Estimated Percent Pig DNA | | | | | | |
| Sample | Cycles | of Reads | M1 | M2 | M3 | M4 | M5 | M6 | M7 |
| Patient sample | 2 × 65 | 36,826,446 | 0.390 | 0.320 | 0.280 | 0.250 | 0.200 | 0.190 | 0.148 |
| Day 6* | | | | | | | | | |
| Patient sample | 2 × 65 | 300,503,918 | 0.440 | 0.340 | 0.200 | 0.250 | 0.190 | 0.190 | 0.163 |
| Day 6⁺ | | | | | | | | | |
| Patient sample | 2 × 65 | 109,730,504 | 0.410 | 0.330 | 0.270 | 0.280 | 0.240 | 0.230 | 0.228 |
| Day 13⁺ | | | | | | | | | |
| Patient sample | 2 × 65 | 106,188,398 | 0.500 | 0.410 | 0.360 | 0.370 | 0.330 | 0.320 | 0.320 |
| Day 19⁺ | | | | | | | | | |
| Patient sample | 2 × 65 | 52,460,056 | 0.860 | 0.780 | 0.720 | 0.690 | 0.620 | 0.600 | 0.585 |
| Day 25* | | | | | | | | | |
| Patient sample | 2 × 35 | 635,272,800 | 0.860 | 0.760 | 0.610 | 0.650 | 0.590 | 0.630 | 0.594 |
| Day 25⁺ | | | | | | | | | |
| Patient sample | 2 × 65 | 40,478,500 | 0.580 | 0.520 | 0.480 | 0.450 | 0.400 | 0.380 | 0.372 |
| Day 33* | | | | | | | | | |
| Patient sample | 2 × 65 | 58,470,038 | 0.474 | 0.408 | 0.371 | 0.343 | 0.288 | 0.266 | 0.254 |
| Day 46* | | | | | | | | | |
| Patient sample | 2 × 65 | 67,310,140 | 0.682 | 0.612 | 0.558 | 0.532 | 0.468 | 0.452 | 0.443 |
| Day 49* | | | | | | | | | |
| Patient sample | 2 × 65 | 64,350,664 | 1.565 | 1.496 | 1.448 | 1.408 | 1.312 | 1.311 | 1.305 |
| Day 55* | | | | | | | | | |
| Patient sample | 2 × 65 | 65,701,052 | 1.093 | 1.023 | 0.970 | 0.936 | 0.850 | 0.841 | 0.833 |
| Day 60* | | | | | | | | | |
| Sheared human | 2 × 55 | 49,445,340 | 0.270 | 0.190 | 0.100 | 0.090 | 0.040 | 0.010 | 0.000 |
| gDNA* | | | | | | | | | |
| Sheared human | 2 × 50 | 18,730,820 | 0.285 | 0.179 | 0.113 | 0.095 | 0.042 | 0.010 | 0.000 |
| gDNA* | | | | | | | | | |
| Sheared pig | 2 × 55 | 51,072,814 | 99.680 | 99.750 | 99.910 | 99.900 | 99.960 | 99.990 | 100.000 |
| gDNA* | | | | | | | | | |
| Human | 2 × 50 | 15,329,378 | 0.270 | 0.180 | 0.100 | 0.090 | 0.040 | 0.010 | 0.000 |
| cfDNA* | | | | | | | | | |
| Human (non- | 2 × 50 | 5,114,174 | 0.119 | 0.088 | 0.000 | 0.028 | 0.000 | 0.000 | 0.000 |
| sheared) | | | | | | | | | |
| gDNA* | | | | | | | | | |
| 0.2% pig | 2 × 65 | 47,651,306 | 0.506 | 0.412 | 0.337 | 0.300 | 0.223 | 0.199 | 0.178 |
| (sheared) | | | | | | | | | |
| control* | | | | | | | | | |
| 0.4% pig | 2 × 65 | 63,487,952 | 0.700 | 0.611 | 0.525 | 0.487 | 0.398 | 0.388 | 0.370 |
| (sheared) | | | | | | | | | |
| control* | | | | | | | | | |

*Sequenced with MiSeq; ⁺Sequenced with NextSeq.

The percent pig-donor-derived cfDNA calculated in samples from the human recipient of the pig heart transplant with either dPCR or shotgun NGS using filtering model M7 were compared. For the digital PCR assays, either singleplex assays with one two-copy pig-specific target and one single-copy human-specific target, or multiplex assays with two single-copy pig-specific targets and one single-copy human-specific target, were used, as described in Table 1.

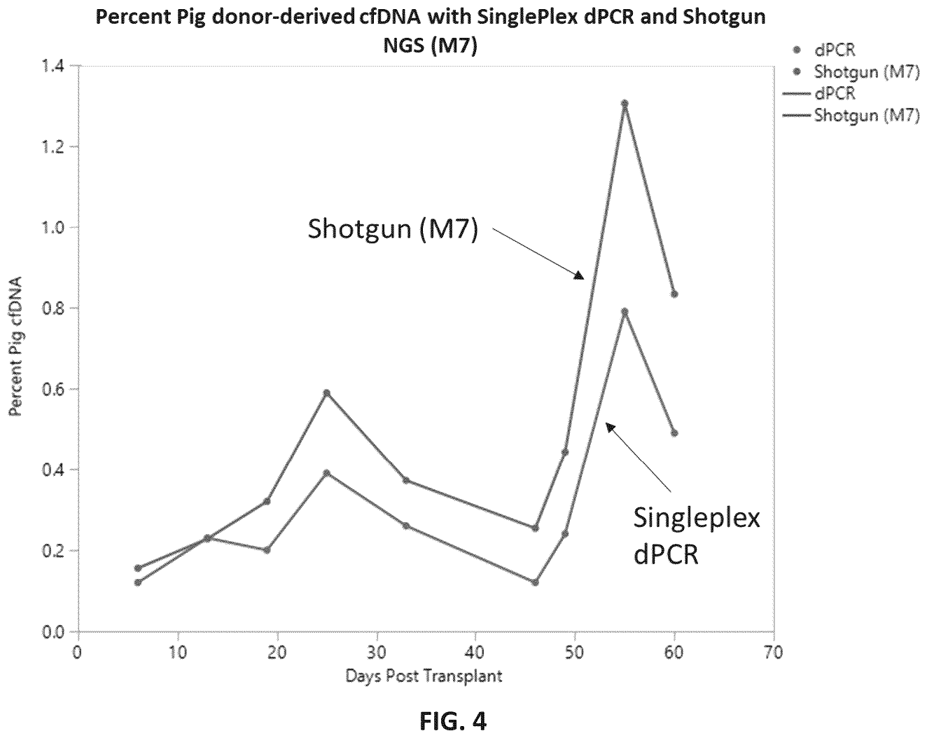

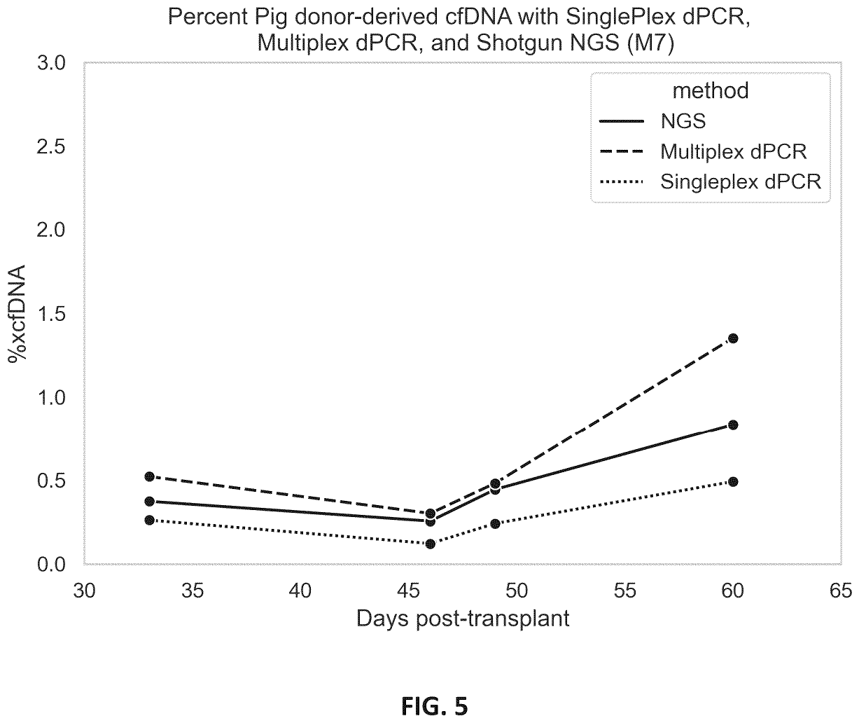

As shown in FIG. 4, FIG. 5 and Table 8, although the determined percentages of pig donor-derived cfDNA were generally different between the dPCR approaches and shotgun NGS, their analyses showed nevertheless a consistent trend over time. As illustrated in FIG. 4, the percent of pig-donor-derived cfDNA was lowest at day 6 after the organ transplantation and reached a peak at day 55 after the organ transplantation, followed by a slight decrease at day 60 after the organ transplantation. The percentage of pig donor-derived cfDNA measured by the above described singleplex dPCR assay with one two-copy pig-specific target and one single-copy human-specific target was two-fold lower than the percentage of pig donor-derived cfDNA measured by the above described shotgun NGS method. The percentage of pig donor-derived cfDNA measured by the above described multiplex dPCR assay with two single-copy pig-specific targets and one single-copy human-specific target was comparable to that measured by the shotgun NGS method (Table 8).

TABLE 8
Comparison of percent of pig donor-derived cell-free DNA (% xcfDNA)
in human recipient of pig heart transplant. The % xcfDNA was measured
by shotgun next-generation sequencing (“NGS”), singleplex dPCR assay
with one two-copy pig-specific target and one single-copy human-specific
target (“singleplex dPCR”), or a multiplex dPCR assay with two
single-copy pig-specific targets and one single-copy human-specific target
(“multiplex dPCR”).

| Days post- | % xcfDNA | % xcfDNA | % xcfDNA |
|---|---|---|---|
| transplant | (NGS) | (singleplex dPCR) | (multiplex dPCR) |
| 33 | 0.372 | 0.26 | 0.52 |
| 46 | 0.254 | 0.12 | 0.30 |
| 49 | 0.443 | 0.24 | 0.48 |
| 60 | 0.833 | 0.49 | 1.35 |

### Example 3: Non-Invasive Short-Term Monitoring of Organ Transplant Health in Human Deceased Model Recipients of a Pig Heart Transplant

This Example describes short-term non-invasive monitoring of organ transplant health in two human deceased model recipients of a pig heart transplant who were briefly kept on artificial life support using the sequencing-based and/or the multiplex dPCR-based methods described in Example 1.

### Samples

Blood samples from two human deceased model recipients, Recipient 1 and Recipient 2, were obtained for quantitation of pig donor-derived cell-free DNA. For Recipient 1, blood was collected in two 10 mL Streck tubes at hours 30, 48, and 72 post-transplantation. For Recipient 2, blood was collected in two 10 mL Streck tubes at hours 0, 12, 24, 36, 48, 60, and 66 post-transplantation; however, since the hour 36 and hour 48 samples were indistinguishable due to smearing of the tube labels, they were excluded from further analysis.

dPCR-Based Analyses

The samples for all usable time points were tested in duplicates using multiplex dPCR reactions with either two single-copy pig assays and one single-copy human assay (“multiplex dPCR #1”), or one multi-copy pig assay and one single-copy human assay (“multiplex dPCR #2”). Input cfDNA amount was 20 ng for multiplex dPCR #1 testing and 10 ng for multiplex dPCR #2 testing. All sample duplicates tested with the multiplex dPCR #2 method showed nearly identical % xcfDNA quantitation with low standard deviations (Table 9).

TABLE 9
Measurement of % xcfDNA, % uncertainty, and porcine genomic copies
per mL of plasma by multiplex dPCR #1 and multiplex dPCR #2,
as well as NGS methods in samples from Recipient 1 and Recipient 2.

| | | | | | | multiplex dPCR #2 | | |
|---|---|---|---|---|---|---|---|---|
| | | | multiplex dPCR #1 | | | | | Porcine |
| | | % | | | Porcine | | | genomic |
| | Hours | xcfDNA | | % | genomic | | % | copies/ |
| | post- | by NGS | % | Uncer- | copies/mL | % | Uncer- | mL of |
| Recipient | transplant | method | xcfDNA | tainty | of plasma | xcfDNA | tainty | plasma |
| 1 | 30 | 2.31 | 6.56 ± | 6.6 ± | 8772.5 ± | 5.12 ± | 5.0 ± | 6318.2 ± |
| | | | 0.18 | 0.1 | 492.0 | 0.14 | 0.3 | 116.2 |
| | 48 | 0.22 | 0.44 ± | 21.2 ± | 1361.4 ± | 0.41 ± | 21.8 ± | 1350.1 ± |
| | | | 0.02 | 1.0 | 84.5 | 0.05 | 1.4 | 158.8 |
| | 72 | 0.1 | 0.29 ± | 33.0 ± | 548.7 ± | 0.24 ± | 32.8 ± | 1475.1± |
| | | | 0.04 | 4.0 | 124.6 | 0.02 | 2.5 | 109.8 |
| 2 | 0 | 0 | 0 ± 0 | n/a | 0 ± 0 | 0 ± 0 | n/a | 0 ± 0 |
| | 12 | 4.42 | 7.74 ± | 10.3 ± | 5954.1 ± | 8.89 ± | 7.1 ± | 5527.1 ± |
| | | | 0.47 | 0.2 | 364.6 | 0.07 | 0.2 | 22.4 |
| | 24 | 3.22 | 7.85 ± | 9.7 ± | 2978.4 ± | 9.93 ± | 6.8 ± | 2814.9 ± |
| | | | 0.27 | 0.4 | 142.5 | 0.40 | 0.1 | 82.2 |
| | 60 | 1.58 | 4.37 ± | 11.9 ± | 2056.5 ± | 3.03 ± | 9.4 ± | 1834.7 ± |
| | | | 0.13 | 0.5 | 150.6 | 0.20 | 0.1 | 22.5 |
| | 66 | 1.22 | 3.87 ± | 13.8 ± | 1768.6 ± | 1.70 ± | 13.2 ± | 1029 ± |
| | | | 0.22 | 1.1 | 202.3 | 0.07 | 0.6 | 69.3 |

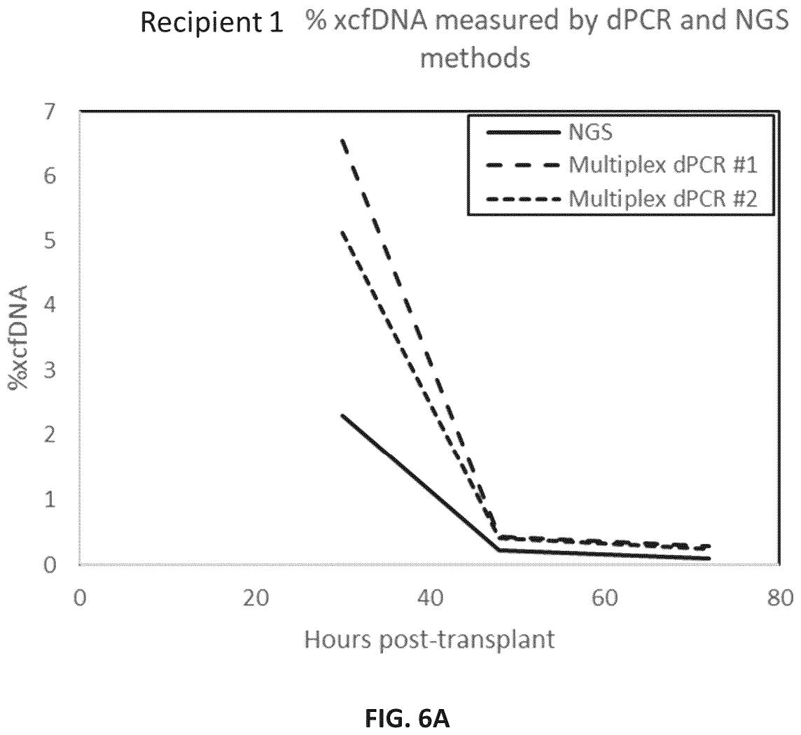

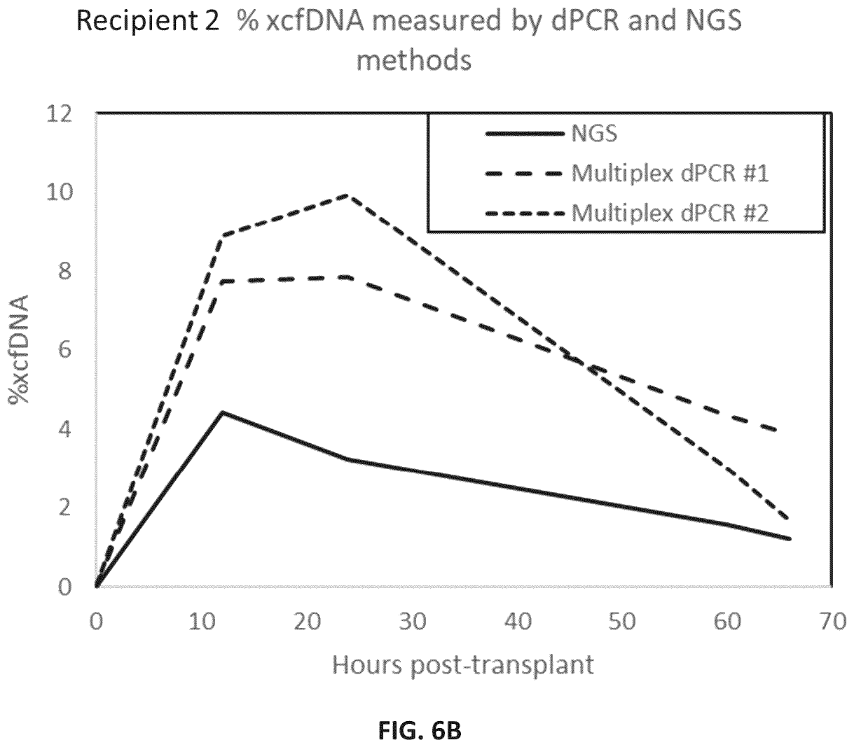

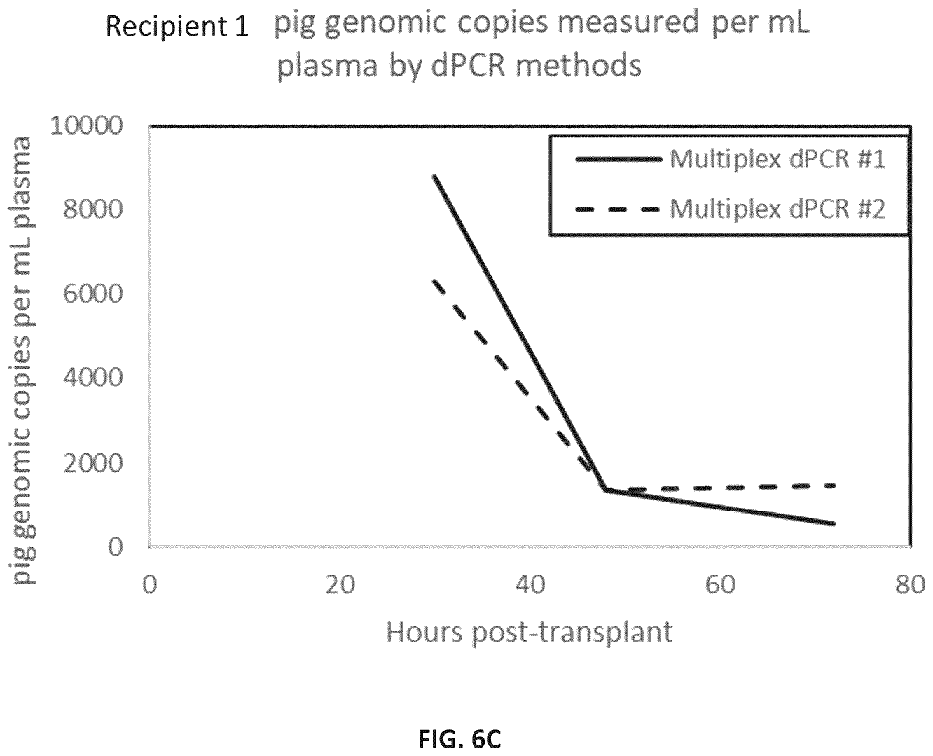

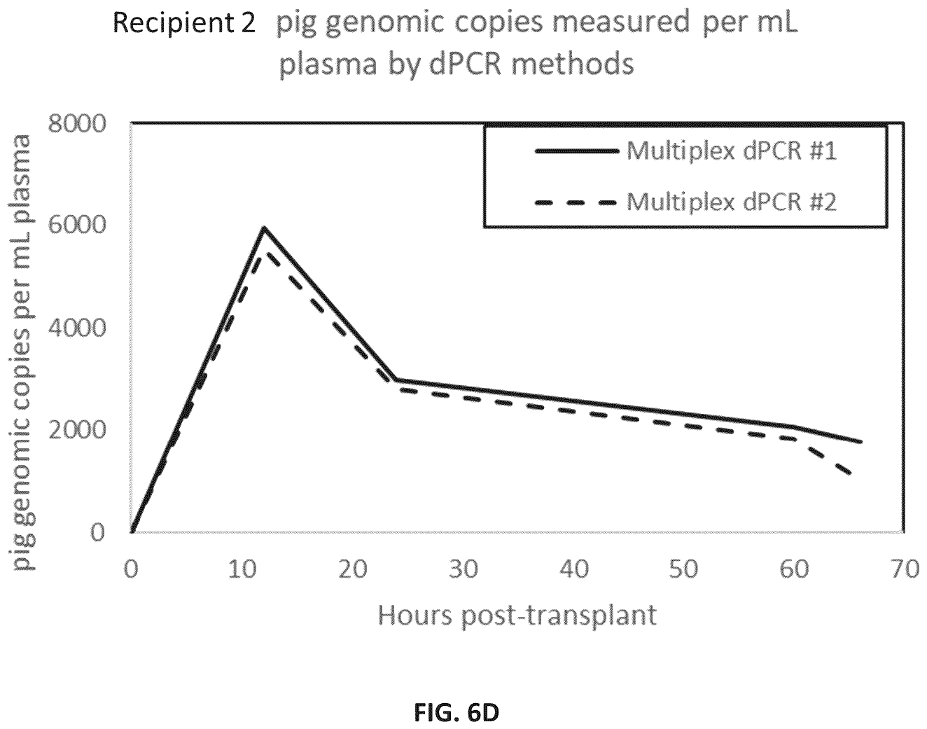

For both Recipients 1 and 2, the % xcfDNA measurements by the multiplex dPCR #1 and multiplex dPCR #2 assays were closer to each other than to the % xcfDNA measurement by NGS (FIGS. 6A & 6B). The porcine genomic copies per mL of plasma measurements by both dPCR methods were also comparable to each other in Recipient 1 samples (FIG. 6C) and almost identical in Recipient 2 (FIG. 6D and Table 9).

1. A method for detecting and monitoring transplant rejection status of a transplant from a donor in a transplant recipient, wherein the donor and the recipient belong to different species, the method comprising:
a) providing a sample from the transplant recipient post-transplant comprising cell-free nucleic acids that are derived from the donor and cell-free nucleic acids that are derived from the recipient;
b) determining an amount of donor-derived cell-free nucleic acids in the sample in a high-throughput sequencing assay by generating sequence reads from the cell-free nucleic acids, wherein the generated sequence reads correspond to donor-specific and recipient-specific genome sequences, and mapping the generated sequence reads to at least the donor-specific genome sequences, wherein differences in genome size between donor and recipient are accounted for; and
c) detecting transplant rejection if the amount of donor-derived cell-free nucleic acids exceeds a predetermined threshold.

2. The method of claim 1, furthermore comprising adding a quantitative spike-in nucleic acid control to the sample in step a, wherein sequence reads from the spike-in control are used to determine the absolute amount of donor-derived cell-free nucleic acids.

3. The method of claim 1 or claim 2, wherein the determining step comprises mapping the generated sequence reads to donor-specific and recipient-specific genome sequences.

4. The method of claim 1, wherein the sequencing reads are generated from regions of selected sizes of the genomes.

5. The method of claim 1, wherein size differences between donor genome and recipient genome are accounted for by utilizing average coverage across regions of selected sizes of the genomes, wherein the regions are large enough to have sufficient coverage and wherein background noise is low enough to differentiate donor and recipient genomes.

6. The method of claim 5, wherein a region has a size of up to 1M bases, up to 10M bases, or up to 100M bases.

7. The method of claim 1, wherein the amount of donor-derived cell free nucleic acids is a percentage of average coverage across regions of selected sizes of the genomes, wherein the regions are large enough to have sufficient coverage and wherein background noise is low enough to differentiate donor and recipient genomes.

8. The method of claim 1, further comprising:
administering an immunosuppressant treatment to the transplant recipient based on the amount of donor-derived cell-free nucleic acids.

9-14. (canceled)

15. The method of claim 1, further comprising:
adjusting immunosuppressant treatment being administered to the transplant recipient based on the amount of donor-derived cell-free nucleic acids.

16-21. (canceled)

22. The method of claim 1, wherein the high-throughput sequencing assay comprises a next-generation sequencing assay.

23. A method for detecting and monitoring transplant rejection status of a transplant from a donor in a transplant recipient, wherein the donor and the recipient belong to different species, the method comprising:
a) providing a sample from the transplant recipient post-transplant, wherein the sample comprises cell-free nucleic acids that are derived from the donor and cell-free nucleic acids that are derived from the recipient;
b) determining an amount of donor-derived cell-free nucleic acids in the sample by a PCR assay, wherein the amount of donor-derived cell-free nucleic acids is determined:
(i) as absolute copies of donor-derived cell-free nucleic acids in the sample, or
(ii) as a ratio of donor-derived cell-free nucleic acids to total donor-derived and recipient-derived cell-free nucleic acids,
by PCR quantitation of donor-specific nucleic acids and recipient-specific nucleic acids; and
c) detecting transplant rejection if the amount of donor-derived cell-free nucleic acids exceeds a predetermined threshold.

24. The method of claim 23, wherein the PCR assay is a quantitative PCR (qPCR) assay and the PCR quantitation is a real time PCR quantitation.

25. The method of claim 23, wherein the PCR assay is a digital PCR assay and the PCR quantitation is an endpoint PCR quantitation.

26. The method of claim 25, wherein the digital PCR assay comprises:
(a) at least a singleplex digital PCR assay for a single-copy or multi-copy donor-specific target, wherein the number of copies of the donor-specific target is in reference to a haploid donor genome; and/or
(b) at least a singleplex digital PCR assay for a single-copy or multi-copy recipient-specific target, wherein the number of copies of the recipient-specific target is in reference to a haploid recipient genome.

27. The method of claim 25, wherein the digital PCR assay comprises at least a multiplex digital PCR assay for two or more single-copy or multi-copy donor-specific targets and/or recipient-specific targets in a single digital PCR reaction, wherein the number of copies of the donor-specific targets is in reference to a haploid donor genome and the number of copies of the recipient-specific targets is in reference to a haploid recipient genome.

28. The method of claim 26, wherein the digital PCR assay comprises a first singleplex digital PCR assay and a second singleplex digital PCR assay, wherein:
(a) the first singleplex digital PCR assay is for a single-copy or multi-copy recipient-specific target, wherein the number of copies of the recipient-specific target is in reference to a haploid recipient genome, and
(b) the second singleplex digital PCR assay is for a single-copy or multi-copy donor-specific target, wherein the number of copies of the donor-specific target is in reference to a haploid donor genome.

29. The method of claim 28, wherein the second singleplex digital PCR assay is for a multi-copy donor-specific target.

30. The method of claim 27, wherein the multiplex digital PCR assay is for:
(a) at least one single-copy or multi-copy recipient-specific target, wherein the number of copies of the recipient-specific target is in reference to a haploid recipient genome, and
(b) at least one single-copy or multi-copy donor-specific target, wherein the number of copies of the donor-specific target is in reference to a haploid donor genome.

31. The method of claim 27 or claim 30, wherein the multiplex digital PCR assay is for at least one multi-copy donor-specific target, wherein the number of copies of the donor specific-target is in reference to a haploid donor genome.

32. The method of claim 23, further comprising:
administering an immunosuppressant treatment to the transplant recipient based on the amount of donor-derived cell-free nucleic acids.

33.-40. (canceled)

41. The method of claim 23, further comprising:
adjusting immunosuppressant treatment being administered to the transplant recipient based on the amount of donor-derived cell-free nucleic acids.

42.-49. (canceled)

50. The method of claim 1 or claim 23, wherein the method further comprises testing for the presence of an infectious agent.

51. The method of claim 50, wherein the infectious agent is selected from the group consisting of viruses, bacteria, fungi, and parasites.

52. The method of claim 51, wherein the infectious agent is a virus selected from the group consisting of Cytomegalovirus, Epstein-Barr virus, Anelloviridae, and BK virus.

53. The method of claim 1 or claim 23, wherein the method further comprises conducting one or more gene expression profiling assays in the recipient.

54. The method of claim 53, wherein a combination score is calculated based on the amount of donor-derived cell-free nucleic acids in the sample and the results of the gene expression profiling assay.

55. A kit for detecting and monitoring transplant rejection status of a transplant from a donor in a transplant recipient, wherein the donor and the recipient belong to different species, the kit comprising:
(a) one or more PCR reaction oligonucleotide primers and probe sets that hybridize to donor-specific or recipient-specific target sequences in cell-free nucleic acids in a sample from the transplant recipient for PCR quantitation of donor-derived cell-free nucleic acids as absolute copies of donor-derived cell-free nucleic acids in the sample, or as a ratio of donor-derived cell-free nucleic acids to total donor-derived and recipient-derived cell-free nucleic acids in the sample, and
(b) instructions for data analysis to determine an amount of donor-derived cell-free nucleic acids.

56. The kit of claim 55, wherein the PCR quantitation is a qPCR quantitation.

57. The kit of claim 55, wherein the PCR quantitation is a digital PCR quantitation.

58. The method of claim 1 or claim 23, or the kit of claim 55, wherein the donor-derived and/or the recipient derived cell-free nucleic acids are DNA.

59.-63. (canceled)

64. The method of claim 1 or claim 23, or the kit of claim 55, wherein the transplant is a solid organ, tissue or cell transplant.

65. The method or kit of claim 64, wherein the donor of the transplant is an animal.

66. The method or kit of claim 65, wherein the animal is a pig.

67. The method of claim 22, wherein the next-generation sequencing assay is amplicon-based.

68. The method of claim 22, wherein the next-generation sequencing assay is non-amplicon based.

69. A method for analyzing a biological sample from a transplant recipient who received a solid organ transplant from a donor, wherein the donor and the recipient belong to different species, the method comprising:
a) isolating cell-free nucleic acids from a biological sample from the transplant recipient, wherein the cell-free nucleic acids comprise cell-free nucleic acids that are derived from the donor and cell-free nucleic acids that are derived from the recipient;
b) generating amplicons by amplifying target regions of the cell-free nucleic acids, wherein the target regions comprise one or more target regions comprising nucleotide sequences that are donor-specific and/or one or more target regions comprising nucleotide sequences that are recipient-specific;
c) generating sequence reads from the generated amplicons by sequencing the amplicons,
wherein the generated sequence reads comprise one or more sequence reads that correspond to donor-specific genome sequences and/or one or more sequence reads that correspond to recipient-specific genome sequences, and
optionally mapping the generated sequence reads to at least donor-specific genome sequences, optionally wherein differences in genome size between donor and recipient are accounted for; and
c) quantifying an amount of cell-free nucleic acids from the generated sequence reads in the sample.

70. The method of claim 69, wherein the amount of cell-free nucleic acids is an amount of transplant donor-derived cell-free nucleic acids in the sample.

71.-72. (canceled)

73. The method of claim 69 or claim 70, further comprising adding one or more quantitative spike-in nucleic acid controls to the sample, and generating sequence reads corresponding the one or more spike-in nucleic acid controls by sequencing the one or more spike-in nucleic acid controls, wherein sequence reads from the spike-in controls are used to determine an absolute amount of transplant donor-derived cell-free nucleic acids in the sample.

74. The method of claim 73, wherein the one or more quantitative spike-in nucleic acid controls are added before step a).

75. The method of claim 73, wherein the one or more quantitative spike-in nucleic acid controls are added after step a).

76. The method of claim 73, wherein the one or more quantitative spike-in nucleic acid controls are added before and after step a).

77. (canceled)

78. A method for analyzing a biological sample from a transplant recipient who received a solid organ transplant from a donor, wherein the donor and the recipient belong to different species, the method comprising:
a) isolating cell-free nucleic acids from a biological sample from the recipient, wherein the cell-free nucleic acids comprise cell-free nucleic acids that are derived from the donor and cell-free nucleic acids that are derived from the recipient;
b) determining an amount of donor-derived cell-free nucleic acids in the sample by a digital PCR assay, wherein the digital PCR assay comprises:
(i) at least one digital PCR assay for one or more single-copy or multi-copy donor-specific targets, or
(ii) at least one digital PCR assay for one or more single-copy or multi-copy recipient-specific targets and one or more single-copy or multi-copy donor-specific targets,
wherein the number of copies of the donor-specific targets is in reference to a haploid donor genome and the number of copies of the recipient-specific targets is in reference to a haploid recipient genome, and
wherein the amount of donor-derived cell-free nucleic acids is determined:
(i) as absolute copies of donor-derived cell-free nucleic acids in the sample, or
(ii) as a ratio of donor-derived cell-free nucleic acids to total donor-derived and recipient-derived cell-free nucleic acids,
by digital PCR quantitation of donor-specific nucleic acids and recipient-specific nucleic acids.

79. The method of claim 78, wherein the digital PCR assay comprises (a) a first digital PCR assay for a single-copy or multi-copy recipient-specific target, wherein the number of copies of the recipient-specific target is in reference to a haploid recipient genome, and (b) a second digital PCR assay for a single-copy or multi-copy donor-specific target, wherein the number of copies of the donor-specific target is in reference to a haploid donor genome.

80. The method of claim 79, wherein the second digital PCR assay is for a multi-copy donor-specific target.

81. The method of claim 78, wherein the digital PCR assay is for (a) at least one single-copy or multi-copy recipient-specific target, wherein the number of copies of the recipient-specific target is in reference to a haploid recipient genome; and (b) at least one single-copy or multi-copy donor-specific target, wherein the number of copies of the donor-specific target is in reference to a haploid donor genome.

82. The method of claim 81, wherein the digital PCR assay is for a multi-copy donor-specific target, wherein the number of copies of the donor-specific target is in reference to a haploid donor genome.
